(19) United States
(12) Patent Application Publication
(10) Pub. No.: US 2007/0141059 A1
(43) Pub. Date: Jun. 21, 2007

(54) Use of cathepsin s inhibitors for treating an immune response caused by administration of a small molecule therapeutic or biologic

(75) Inventor: Kyle C. Elrod, Belmont, CA (US)

Correspondence Address: MILLEN, WHITE, ZELANO AND BRANIGAN, P.C.;C/O BERLEX BIOSCIENCES, 2200 CLARENDON BOUEVARD, SUITE 1400, ARLINGTON, VA, 22201, US

(73) Assignee: AXYS Pharmaceuticals, Inc., South San Francisco, CA (US)

(21) Appl. No.: 10/581,977

(22) PCT Filed: Dec. 10, 2004
(86) PCT No.: PCT/US04/41580

§ 371 Date: Oct. 19, 2006

Related U.S. Application Data
(60) Provisional application No. 60/528,846, filed on Dec. 11, 2003.
(60) Provisional application No. 60/532,202, filed on Dec. 23, 2003.

(51) Int. Cl. A61K 39/395; A61K 31/727; A61K 38/21; A61K 39/08; A61K 31/165

(52) U.S. Cl. 424/146.1; 424/239.1; 424/85.6; 424/85.7; 514/56; 514/617

(57) ABSTRACT

The present invention is directed to the use of Cathepsin S inhibitors in combination with a therapy that causes a deleterious immune response in patients receiving the therapy.

### FIELD OF INVENTION

The present invention is directed to the use of Cathepsin S inhibitors in combination with a therapy that causes a deleterious immune response in patients receiving the therapy.

### STATE OF THE ART

As a general rule, the therapeutics approved by regulatory agencies for prescription use are safe. Measurement of safety is a key requirement for U.S. approval and is stringently monitored during and after clinical trials. However, safety is a relative term since the benefit of a therapy can outweigh an unintended side effect. Thus, many therapeutic agents are approved and in use today with known side-effect profiles. Such profiles range from minor irritations such as injection site discoloration to a measurable risk of death. The present invention addresses a specific complication that is incurred from multiple therapies i.e., a deleterious immune response caused by the therapies.

As an example, there are many examples of biological agents that induce the production of specific immunoglobulins. These immunoglobulins bind with one or more epitopes on the biological agents thereby rendering the biological agents less effective in many cases. For example, 10% of patients taking Remicade® generate neutralizing antibodies, 45% of patients taking Betaseron® were found to have serum-neutralizing activity, and 25% of patients taking Referon®-A generated neutralizing antibodies. It was recently reported that neutralizing antibodies against interferon Beta which is used for treating multiple sclerosis reduces the clinical effect of the drug (see Lancet, dated Oct. 11, 2003). In fact, host immune response prevents effective application of retroviral therapy (see Journal of Virology 72, 2388-2397, European Journal of Immunology 27, 653-59). A side effect of antibody binding is the activation of humoral or cell-mediated defenses (e.g., complement, mast cells, and macrophages). Therefore, a second consequence of antibody production is the generation of a host reaction that can be deleterious to the patient. Such reactions include inflammation at the site of injection, the binding of neutralizing antibodies to the host's own proteins or in the case of repeated exposure a lethal systemic anaphylaxis. As an example, a number of patients taking Epogen® exhibit pure red cell aplasia as consequence of antibodies generated in response to administration of Epogen®.

Small molecule drugs can also induce a deleterious immune response. For example, drug induced lupus affects, 30,000 to 50,0000 patients in the US. While not fully understood, lupus is meadiated by IgG and the compounds that induce lupus are believed to form protein conjugates that stimulate an immune reaction (see: Rheumatology 2^{nd} Edition, Klippel, J. H. et al. eds., Mosby, Chapter 7 pp 36.4-36.5 and package insert Pronestyl). In another example, patients taking heparin may experience heparin induced thrombocytopenia (HIT). This occurs in ˜3% of patients and represents a significant medical problem prompting research into alternative therapies.

Currently, these problems are most commonly being handled by ceasing the administration of the therapeutic agent. In the cases of biologic agents, alternative solutions include administering methotrexate along with a biologic, increasing the dose of the biologic to induce tolerance or even reengineering the biologic agent to reduce immunogenicity. All these approaches are undesirable; desisting medical therapy requires a replacement therapy or leaves an unmet medical need, methotrexate is associated with severe side effects such as liver damage, nerve damage, kidney damage, and hair loss, utilizing higher doses of the biologic to induce tolerance or reengineering a biologic increases drug development costs and medical expenses.

Accordingly, there is a need for therapeutic agents that can be co-administered with a therapy that generates an immune response in the patient receiving the therapy to prevent or ameroliate the generation of such immune response in the patient.

The present invention fulfills this and related needs.

### DETAILED DESCRIPTION

This invention is directed to a method of treating a patient undergoing a non-tissue graft therapy wherein the therapy may or does induce a deleterious immune response in the patient comprising administering to the patient a Cathepsin S inhibitor. Preferably, a method of treating a patient undergoing a non-tissue graft therapy wherein the therapy induces a deleterious immune response in the patient comprising administering to the patient a Cathepsin S inhibitor. Preferably, the immune response is mediated by MHC Class II molecules. The Cathepsin S inhibitor can be administered prior to, simultaneously, or after treatment of the patient with the therapy. Preferably, the therapy involves treatment of the patient with a biologic or a small molecule therapeutic wherein the biologic or the small molecule therapeutic causes a deleterious immune response in the patient.

In a second aspect, this invention is directed to a method of treating immune response in an animal that is caused by administration of a small molecule therapeutic or a biologic to the animal which method comprises administering to the animal in need of such treatment a therapeutically effective amount of a Cathepsin S inhibitor. Preferably, the immune response is caused by administration of a biologic to the animal. Preferably, the animal is human.

In a third aspect, this invention is directed to a method of prophylactically treating an t immune response in a patient caused by administration of a small molecule therapeutic or a biologic to the patient which method comprises administering to the patient a Cathepsin S inhibitor. Preferably, the immune response is caused by administration of a biologic to the patient.

In a fourth aspect, this invention is directed to a method of improving efficacy of a biologic in an animal comprising administering the biologic to the animal with a Cathepsin S inhibitor. Preferably, the animal is human.

In a fifth aspect, this invention is directed to a method of conducting a clinical trial for a biologic comprising administering to an individual participating in the clinical trial a Cathepsin S inhibitor with the biologic.

In a sixth aspect, this invention is directed to a method of determining the loss in the efficacy of a biologic in an animal due to the immune response caused by the biologic comprising administering the biologic to the animal in the presence and absence of a Cathepsin S inhibitor.

In a seventh aspect, this invention is directed to the use of a Cathepsin S inhibitor for the manufacture of a medicament for combination therapy with a biologic. Specifically, use of a Cathepsin S inhibitor for the manufacture of a medicament for combination therapy with a biologic wherein the Cathepsin inhibitor treats the immune response caused by the biologic.

In an eighth aspect, this invention is directed to a method of treating a patient undergoing treatment with a biologic wherein the biologic causes a deleterious immune response in the patient comprising administering to the patient a Cathepsin S inhibitor.

Preferably, the biologic is a protein. More preferably the biologic is an antibody, preferably a monoclonal antibody. More preferrably, the biologic is Remicade®, Refacto®, Referon-A®, Factor VIII, Factor VII, Betaseron®, Epogen®, Embrel®, Interferon beta, Botox®, Fabrazyme®, Elspar®, Cerezyme®, Myobloc®, Aldurazyme®, Verluma®, Interferon alpha, Humira®, Aranesp®, Zevalin® or OKT3.

Preferably, the small molecule therapeutic is heparin, low molecular weight heparin, procainamide, or hydralazine.

Preferably, the Cathepsin S inhibitor is:
(a) a compound of Formula (Ia) or (Ib): 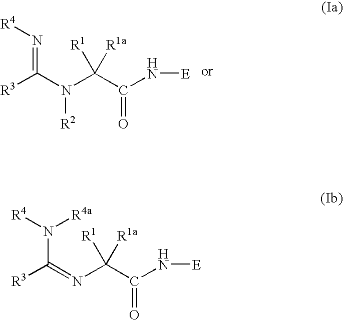
wherein:

E is:
(i) —C(R⁵)(R⁶)X¹ where X¹ is —CHO, —C(R⁷)(R⁸)CF₃, —C(R⁷)(R⁸)CF₂CF₂R⁹, —C(R⁷)(R⁸)R¹⁰, —C(O)C(O)R¹⁰, —CH═CHS(O)₂R¹⁰, —C(R⁷)(R⁸)C(R⁷)(R⁸)OR¹⁰, —C(R⁷)(R⁸)CH₂OR¹⁰, —C(R⁷)(R⁸)C(R⁷)(R⁸)R¹⁰, —C(R⁷)(R⁸)CH₂N(R¹¹)SO₂R¹⁰, —C(R⁷)(R⁸)CF₂C(O)NR¹⁰R¹¹, —C(R⁷)(R⁸)C(O)NR¹⁰R¹¹, —C(R⁷)(R⁸)C(O)N(R¹¹)(CH₂)₂OR¹¹, or —C(R⁷)(R⁸)C(O)N(R¹¹)(CH₂)₂NR¹⁰R¹¹, or
(ii) —C(R^{5a})(R^{6a})CN;
   where:

R⁵ and R^{5a} are independently hydrogen or alkyl;

R⁶ and R^{6a} are independently selected from the group consisting of hydrogen, alkyl, haloalkyl, cycloalkyl, cycloalkylalkyl, aryl, aralkyl, heteroaryl, heteroaralkyl, heterocycloalkyl, heterocycloalkylalkyl, -alkylene-X—R¹² (where X is —O—, —NR¹³—, —S(O)ₙ₁—, —CONR¹³—, —NR¹³CO—, —NR¹³C(O)O—, —NR¹³CONR¹³—, —OCONR¹³—, —NR¹³SO₂—, —SO₂NR¹³—, —NR¹³SO₂NR¹³—, —CO—, —OCO—, or —C(O)O— where n1 is 0-2, R¹² hydrogen, alkyl, haloalkyl, cycloalkyl, cycloalkylalkyl, heterocycloalkyl, heterocycloalkylalkyl, aryl, aralkyl, heteroaryl, or heteroaralkyl and each R¹³ is hydrogen or alkyl) wherein the aromatic or alicyclic ring in R⁶ and R^{6a} is optionally substituted with one, two, or three R^{a} independently selected from alkyl, haloalkyl, alkoxy, hydroxy, haloalkoxy, halo, carboxy, alkoxycarbonyl, amino, monsubstituted amino, disubstituted amino, nitro, aryloxy, benzyloxy, acyl, alkylsulfonyl, or arylsulfonyl where the aromatic or alicyclic ring in R^{a} is optionally substituted with one or two substituents independently selected from alkyl, halo, alkoxy, haloalkyl, haloalkoxy, hydroxy, amino, alkylamino, dialkylamino, carboxy, or alkoxycarbonyl; or

R⁵ and R⁶ and R^{5a} and R^{6a} taken together with the carbon atom to which both R⁵ and R⁶ and R^{5a} and R^{6a} are attached form (i) cycloalkylene optionally substituted with one or two R^{b} independently selected from alkyl, halo, alkylamino, dialkylamino, aryl, aralkyl, cycloalkyl, cycloalkylalkyl, heteroaryl, heteroaralkyl, alkoxycarbonyl, or aryloxycarbonyl or (ii) heterocycloalkylene optionally substituted with one to four alkyl or one or two R^{c} independently selected from alkyl, haloalkyl, hydroxy, hydroxyalkyl, alkoxyalkyl, alkoxyalkyloxyalkyl, aryloxyalkyl, heteroaryloxyalkyl, aminoalkyl, acyl, aryl, aralkyl, heteroaryl, heteroaralkyl, heterocycloalkyl, heterocycloalkylalkyl, cycloalkyl, cycloalkylalkyl, —S(O)ₙ₂R¹⁴, -alkylene-S(O)ₙ₂—R¹⁵, —COOR¹⁶, -alkylene-COOR¹⁷, —CONR¹⁸R¹⁹, or -alkylene-CONR²⁰R²¹ (where n2 is 0-2 and R¹⁴-R¹⁷, R¹⁸ and R²⁰ are independently hydrogen, alkyl, haloalkyl, aryl, aralkyl, heteroaryl, heteroaralkyl, cycloalkyl, cycloalkylalkyl, or heterocycloalkyl and R¹⁹ and R²¹ are independently hydrogen or alkyl) wherein the aromatic or alicyclic ring in the groups attached to cycloalkylene or heterocycloalkylene is optionally substituted with one, two, or three substituents independently selected from alkyl, haloalkyl, cycloalkyl, cycloalkylalkyl, benzyl, alkoxy, hydroxy, haloalkoxy, halo, carboxy, alkoxycarbonyl, amino, monsubstituted amino, disubstituted amino, or acyl;

R⁷ is hydrogen or alkyl;

R⁸ is hydroxy; or

R⁷ and R⁸ together form oxo;

R⁹ is hydrogen, halo, alkyl, aralkyl or heteroaralkyl;

R¹⁰ is alkyl, aryl, aralkyl, heteroaryl, heteroaralkyl, cycloalkyl, cycloalkylalkyl, heterocycloalkyl, or heterocycloalkylalkyl wherein the aromatic or alicyclic ring in R¹⁰ is optionally substituted with one, two, or three R^{d} independently selected from alkyl, haloalkyl, alkoxy, alkoxyalkyl, cycloalkyl, hydroxy, haloalkoxy, halo, carboxy, alkoxycarbonyl, aminosulfonyl, alkylsulfonyl, arylsulfonyl, heteroarylsulfonyl, aryl, aralkyl, heteroaryl, amino, monsubstituted amino, disubstituted amino, carbamoyl, or acyl wherein the aromatic or alicyclic ring in R^{d} is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino; and

R¹¹ is hydrogen or alkyl; or
(iii) a group of formula (a): 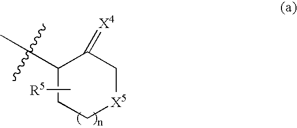
   where:

n is 0, 1, or 2;

X⁴ is selected from —NR²²—, —S—, or —O— where R²² is hydrogen, alkyl, or alkoxy; and

X⁵ is —O—, —S—, —SO₂—, or —NR²³—where R²³ is selected from hydrogen, alkyl, haloalkyl, hydroxyalkyl, alkoxyalkyl, aryloxyalkyl, heteroaryloxyalkyl, aminoalkyl, acyl, aryl, aralkyl, heteroaryl, heteroaralkyl, cycloalkyl, cycloalkylalkyl, —S(O)₂R²⁴, -alkylene-S(O)ₙ₃—R²⁵, —COOR²⁶, -alkylene-COOR²⁷, —CONR²⁸R²⁹, or -alkylene-CONR³⁰R³¹ (where n3 is 0-2 and R²⁴—R²⁷, R²⁸ and R³⁰ are independently hydrogen, alkyl, haloalkyl, aryl, aralkyl, heteroaryl, heteroaralkyl, cycloalkyl, cycloalkylalkyl, heterocycloalkyl, or heterocycloalkylalkyl and R²⁹ and R³¹ are independently hydrogen or alkyl) where the aromatic or alicyclic ring in X⁵ is optionally substituted with one, two, or three substituents independently selected from alkyl, haloalkyl, alkoxy, haloalkoxy, halo, hydroxy, amino, alkylamino, dialkylamino, carboxy, or alkoxycarbonyl and one substitutent selected from aryl, aralkyl, heteroaryl, or heteroaralkyl;

R⁵ is as defined above;

R¹ is hydrogen or alkyl;

R^{1a} is hydrogen, alkyl, haloalkyl, cycloalkyl, cycloalkylalkyl, aryl, aralkyl, heteroaryl, heteroaralkyl, heterocycloalkylalkyl, or -alkylene-X²—R³² [wherein X² is —NR³³—, —O—, —S(O)ₙ₄—, —CO—, —COO—, —OCO—, —NR³³CO—, —CONR³³—, —NR³³SO₂—, —SO₂NR³³—, —NR³³COO—, —OCONR³³—, —NR³³CONR³⁴, or —NR³³SO₂NR³⁴— (where R³³ and R³⁴ are independently hydrogen, alkyl, or acyl and n4 is 0-2) and R³² is hydrogen, alkyl, haloalkyl, cycloalkyl, cycloalkylalkyl, heterocycloalkyl, aryl, aralkyl, heteroaryl, heteroaralkyl, heterocycloalkyl, or heterocycloalkylalkyl] wherein said alkylene chain is optionally substituted with one to six halo and wherein the aromatic or alicyclic ring in R^{1a} is optionally substituted with one, two, or three R^{e} independently selected from alkyl, haloalkyl, alkoxy, alkylthio, alkylsulfonyl, arylsulfonyl, aminocarbonyl, aminosulfonyl, acyl, hydroxy, haloalkoxy, halo, nitro, cyano, carboxy, alkoxycarbonyl, aryloxycarbonyl, aryl, heteroaryl, cycloalkyl, cycloalkylalkyl, aralkyl, heteroaralkyl, amino, monsubstituted amino, disubstituted amino, or acyl; or

R¹ and R^{1a} together with the carbon atoms to which they are attached form cycloalkylene or heterocycloalkylene ring wherein said cycloalkylene or heterocycloalkylene is optionally substituted with one or two R^{f} independently selected from alkyl, halo, haloalkyl, hydroxyalkyl, keto, or —SO₂R where R is alkyl, cycloalkyl, cycloalkylalkyl, aryl, aralkyl, heteroaryl or heteroaralkyl where the aromatic or alicylic ring in R^{f} is optionally substituted with one, two, or three substitutents independently selected from alkyl, alkoxy, haloalkyl, haloalkoxy, hydroxy, halo, carboxy, or alkoxycarbonyl;

R² is hydrogen or alkyl;

R³ is hydrogen, alkyl, haloalkyl, cycloalkyl, aryl, aralkyl, heteroaryl, heteroaralkyl, heterocycloalkyl, heterocycloalkylalkyl, amino, mono or disubstituted amino, or -alkylene-X³—R³⁵ [wherein X³ is —NR³⁶—, —O—, —S(O)ₙ₅—, —CO—, —COO—, —OCO—, —NR³⁶CO—, —CONR³⁶—, —NR³⁶SO₂—, —SO₂NR³⁶—, —NR³⁶COO—, —OCONR³⁶—, —NR³⁶CONR³⁷—, or —NR³⁶SO₂NR³⁷— (where R³⁶ and R³⁷ are independently hydrogen, alkyl, or acyl and n5 is 0-2) and R³⁵ is hydrogen, alkyl, haloalkyl, cycloalkyl, cycloalkylalkyl, aryl, aralkyl, heteroaryl, or heteroaralkyl] wherein the aromatic or alicyclic rings in R³ are optionally substituted by one, two, or three R^{g} independently selected from alkyl, halo, hydroxy, alkoxy, haloalkyl, haloalkoxy, oxo, cyano, nitro, acyl, acyloxy, aryl, heteroaryl, cycloalkyl, heterocycloalkyl, aryloxy, benzyloxy, carboxy, alkoxycarbonyl, aryloxycarbonyl, carbamoyl, alkylthio, alkylsulfinyl, alkylsulfonyl, arylthio, arylsulfonyl, arylsulfinyl, alkoxycarbonylamino, aryloxycarbonylamino, alkylcarbamoyloxy, arylcarbamoyloxy, alkylsulfonylamino, arylsulfonylamino, aminosulfonyl, alkylaminosulfonyl, dialkylaminosulfonyl, arylaminosulfonyl, amino, monosubsituted or disubstituted amino, and further wherein the aromatic and alicyclic rings in R^{g} are optionally substituted with one, two, or three R^{h} wherein R^{h} is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, alkylamino, dialkylamino, aryl, heteroaryl, cycloalkyl, carboxy, carboxamido, or alkoxycarbonyl;

R⁴ is hydrogen, alkyl, hydroxy, nitrile, or -(alkylene)n₆-X⁶—R³⁸ (where X⁶ is —O—, —NR³⁹—, —S(O)ₙ₇—, —NR³⁹CO—, —CO—, or —OC(O)— where n6 is 0 or 1, n7 is 0-2, and R³⁹ is hydrogen or alkyl) and R³⁸ is hydrogen, alkyl, phenyl, naphthyl, pyrrolidinyl, piperidinyl, morpholinyl, thiomorpholinyl, piperazinyl, indolinyl, pyranyl, thiopyranyl, furanyl, thienyl, pyrrolyl, oxazolyl, isoxazolyl, thiazolyl, imidazolyl, pyridinyl, pyrimidinyl, pyrazinyl, indolyl, benzofuranyl, benzothienyl, benzimidazolyl, benzthiazolyl, quinolinyl, isoquinolinyl, quinazolinyl, benzoxazolyl, or quinoxalinyl where R³⁸ is optionally substituted with one, two, or three Rⁱ independently selected from alkyl, alkoxy, halo, haloalkyl, haloalkoxy, hydroxy, alkylthio, alkylsulfonyl, arylsulfonyl, aminosulfonyl, acyl, amino, monosubstituted amino, disubstituted amino, carboxy, alkoxycarbonyl, hydroxyalkyl, alkoxyalkyl, aminoalkyl, aryl, heteroaryl, or heterocycloalkyl where the aromatic or alicyclic ring in Rⁱ is optionally substituted with one or two substituents independently selected from alkyl, halo, alkoxy, haloalkyl, haloalkoxy, hydroxy, amino, alkylamino, dialkylamino, carboxy, or alkoxycarbonyl; or

R³ and R⁴ in (Ia) or (Ib) together with the atoms to which they are attached form heteroaryl or heterocycloalkyl ring optionally fused to an aryl or heteroaryl ring wherein said rings are optionally substituted on the aromatic and/or non-aromatic portion of the rings with one, two, or three R^{j};

each R^{j} and R^{4a} is independently:

hydrogen, alkyl optionally interrupted by one or two N, O, C(O), S, S(O), or S(O)₂ and optionally substituted by amino, hydroxy, halo, alkyl, pyrrolidinyl, piperidinyl, morpholinyl, thiomorpholinyl, piperazinyl, indolinyl, pyranyl, thiopyranyl, furanyl, thienyl, pyrrolyl, oxazolyl, isoxazolyl, thiazolyl, imidazolyl, pyridinyl, pyrimidinyl, pyrazinyl, indolyl, benzofuranyl, benzothienyl, benzimidazolyl, benzthiazolyl, quinolinyl, isoquinolinyl, quinazolinyl, benzoxazolyl or quinoxalinyl;

halo, alkoxy, alkylthio, hydroxy, carboxy, aryl, aryloxy, aroyl, heteroaryl, alkanoyl, —C(O)OR where (R is hydrogen, alkyl, alkoxyalkyl, haloalkyl, cycloalkyl, cycloalkylalkyl, heteroaryl, heteroarylalkyl, aryl, arylalkyl, aminoalkyl, heterocycloalkyl, or heterocycloalkylalkyl), aminocarbonyl, aminosulfonyl, alkylsulfonyl, aryloxycarbonyl, benzyloxycarbonyl, alkanoylamino, alkylaminocarbonyl, dialkylaminocarbonyl, alkoxycarbonylamino, aroylamino, amino, alkylamino, dialkylamino, alkylthio, arylthio, alkylsulfonylamino, arylsulfonylamino, alkylaminosulfonyl, arylaminosulfonyl, cycloalkyl, benzyloxy, or ureido wherein each of the aforementioned groups in R^{4a} and R^{j} is optionally substituted with one, two, or three substituents independently selected from halo, hydroxy, alkyl, alkoxy, haloalkyl, haloalkoxy, oxo, carboxy, nitrile, nitro or NH₂C(O)—; or
(b) a compound of Formula (II): 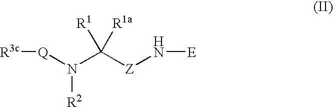
where:

E, R¹, R^{1a} and R² are as defined above;

Z is —CO— or —CH₂SO₂—; or

Q is —CO—, —SO₂—, —OCO—, —NRCO—, or —NRSO₂— where R is hydrogen, alkyl, hydroxyalkyl, alkoxyalkyl, or aralkyl;

R^{3c} is alkyl, haloalkyl, cycloalkyl, cycloalkylalkyl, aryl, aralkyl, heteroaryl, heteroaralkyl, heterocycloalkyl, heterocycloalkylalkyl, or -alkylene-X⁸—R⁴⁰ [wherein X⁸ is —NR⁴¹—, —O—, —S(O)ₙ₈—, —CO—, —COO—, —OCO—, —NR⁴¹CO—, —CONR⁴¹—, —NR⁴¹SO₂—, —SO₂NR⁴¹—, —NR⁴¹COO—, —OCONR⁴¹—, —NR⁴¹CONR⁴²—, or —NR⁴¹SO₂NR⁴²— (where each R⁴¹ and R⁴² is independently hydrogen, alkyl, or acyl and n8 is 0-2) and R⁴⁰ is hydrogen, alkyl, haloalkyl, cycloalkyl, cycloalkylalkyl, heterocycloalkyl, heterocycloalkylalkyl, aryl, aralkyl, heteroaryl, or heteroaralkyl] wherein the alkylene chain in R^{3c} is optionally substituted with one to three halo atoms and the aromatic and alicyclic rings in R^{3c} are optionally substituted by one, two, or three R^{k} independently selected from alkyl, aminoalkyl, halo, hydroxy, alkoxy, haloalkyl, haloalkoxy, oxo, cyano, nitro, acyl, acyloxy, aryl, aralkyl, heteroaryl, heteroaralkyl, cycloalkyl, cycloalkylalkyl, heterocycloalkyl, heterocycloalkylalkyl, aryloxy, benzyloxy, carboxy, alkoxycarbonyl, aryloxycarbonyl, carbamoyl, alkylthio, alkylsulfinyl, alkylsulfonyl, arylthio, arylsulfonyl, arylsulfinyl, alkoxycarbonylamino, aryloxycarbonylamino, alkylcarbamoyloxy, arylcarbamoyloxy, alkylsulfonylamino, arylsulfonylamino, aminosulfonyl, alkylaminosulfonyl, dialkylaminosulfonyl, arylaminosulfonyl, aralkylaminosulfonyl, aminocarbonyl, arylaminocarbonyl, aralkylaminocarbonyl, amino, monosubsituted or disubstituted amino, and further wherein the aromatic and alicyclic rings in R^{k} are optionally substituted with one, two, or three R¹ wherein R¹ is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, monosubstituted amino, dialkylamino, aryl, heteroaryl, cycloalkyl, carboxy, carboxamido, or alkoxycarbonyl; or
(c) a compound of Formula (III): 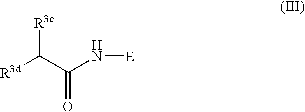
where E is as defined above;

R^{3d} and R^{3e} are independently -alkylene-X⁹—R⁴³ [wherein X⁹ is bond, —NR⁴⁴—, —O—, —S(O)ₙ₉—, —CO—, —COO—, —OCO—, —NR⁴⁴CO—, —CONR⁴⁴—, —NR⁴⁴SO₂—, —SO₂NR⁴⁴—, —NR⁴⁴COO—, —OCONR⁴⁴—, —NR⁴⁴CONR⁴⁵—, or —NR⁴⁴SO₂NR⁴⁵— (where R⁴⁴ and R⁴⁵ are independently hydrogen, alkyl, or acyl and n9 is 0-2) and R⁴³ is hydrogen, alkyl, haloalkyl, cycloalkyl, cycloalkylalkyl, aryl, aralkyl, heteroaryl, heteroaralkyl, heterocycloalkyl, or heterocycloalkylalkyl] wherein the alkylene chain is optionally substituted with one to three halo atoms and the aromatic or alicyclic rings in R^{3d} and R^{3e} are optionally substituted by one, two, or three R^{m} independently selected from alkyl, halo, hydroxy, alkoxy, haloalkyl, haloalkoxy, oxo, cyano, nitro, acyl, acyloxy, carboxy, alkoxycarbonyl, carbamoyl, alkylthio, alkylsulfinyl, alkylsulfonyl, alkoxycarbonylamino, alkylcarbamoyloxy, alkylsulfonylamino, aminosulfonyl, alkylaminosulfonyl, dialkylaminosulfonyl, aminocarbonyl, amino, monosubsituted or disubstituted amino and one R^{m} selected from aryl, aralkyl, heteroaryl, heteroaralkyl, cycloalkyl, cycloalkylalkyl, heterocycloalkyl, heterocycloalkylalkyl, aryloxy, benzyloxy, aryloxycarbonyl, arylthio, arylsulfonyl, arylsulfinyl, aryloxycarbonylamino, arylcarbamoyloxy, arylsulfonylamino, arylaminosulfonyl, or aralkylaminosulfonyl wherein the aromatic or alicyclic ring in R^{m} is optionally substituted with one, two, or three Rⁿ wherein Rⁿ is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, alkylsulfonylamino, arylsulfonylamino, heteroarylsulfonylamino, heteroaralkylsulfonylamino, amino, monosubstituted amino, dialkylamino, aryl, heteroaryl, cycloalkyl, carboxy, carboxamido, or alkoxycarbonyl; or
(d) a compound of Formula (IV): 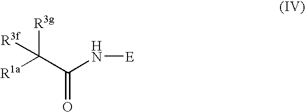
where:

E and R^{1a} are as defined above;

R^{3f} is hydrogen;

R^{3g} is hydrogen, fluoro, —OR⁴⁶ or —NR⁴⁷R⁴⁸ where:

R⁴⁶ is hydrogen, alkyl, cycloalkyl, cycloalkylalkyl, aryl, aralkyl, heteroaryl, heteroaralkyl, heterocycloalkyl, heterocycloalkylalkyl, -(alkylene)n₁₀-X¹⁰—R⁴⁹ [wherein n10 is 0 or 1, X¹⁰ is —CO— or —CONR⁵⁰— where R⁵⁰ is hydrogen, alkyl, or alkoxyalkyl, and R⁴⁹ is hydrogen, alkyl, haloalkyl, alkoxyalkyl, hydroxyalkyl, aryl, aralkyl, cycloalkyl, cycloalkylalkyl, heteroaryl, heteroaralkyl, heterocycloalkyl or heterocycloalkylalkyl or R⁴⁹ and R⁵⁰ together with the nitrogen atom to which they are attached from heterocycloalkyl], or -alkylene-X¹¹—R⁵¹ [wherein X¹¹ is —NR⁵²—, —O—, —S(O)ₙ₁₁—, —COO—, —OCO—, —NR⁵²CO—, —NR⁵²SO₂—, —SO₂NR⁵²—, —NR⁵²COO—, —OCONR⁵²—, —NR⁵²CONR⁵³—, or —NR⁵²SO₂NR⁵³— where n11 is hydrogen or alkyl, R⁵² is hydrogen or alkyl, and R⁵¹ is hydrogen, alkyl, haloalkyl, alkoxyalkyl, hydroxyalkyl, cycloalkyl, cycloalkylalkyl, heterocycloalkyl, heterocycloalkylalkyl, aryl, aralkyl, heteroaryl, heteroaralkyl or heterocycloalkylalkyl or R⁵¹ together with R⁵² or R⁵³ in —SO₂NR⁵²—, —OCONR⁵²—, —NR⁵²CONR⁵³—, or —NR⁵²SO₂NR⁵³— form heterocycloalkyl] wherein the alkylene chain is optionally substituted with one to three halo atoms and the aromatic or alicyclic rings in R⁴⁶ are optionally substituted by one, two, or three R^{o} independently selected from alkyl, halo, hydroxy, alkoxy, hydroxyalkyl, alkoxyalkyl, haloalkyl, haloalkoxy, oxo, cyano, nitro, acyl, acyloxy, carboxy, alkoxycarbonyl, carbamoyl, alkylthio, alkylsulfinyl, alkylsulfonyl, alkoxycarbonylamino, alkylcarbamoyloxy, alkylsulfonylamino, aminosulfonyl, alkylaminosulfonyl, dialkylaminosulfonyl, aminocarbonyl, amino, monosubsituted or disubstituted amino and one R^{o} selected from aryl, aralkyl, heteroaryl, heteroaralkyl, cycloalkyl, cycloalkylalkyl, heterocycloalkyl, heterocycloalkylalkyl, aryloxy, benzyloxy, aryloxycarbonyl, arylthio, arylsulfonyl, arylsulfinyl, aryloxycarbonylamino, arylcarbamoyloxy, arylsulfonylamino, arylaminosulfonyl, or aralkylaminosulfonyl wherein the aromatic and alicyclic rings in R^{o} are optionally substituted with one, two, or three R^{p} wherein R^{p} is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, monosubstituted amino, dialkylamino, aryl, heteroaryl, cycloalkyl, carboxy, carboxamido, or alkoxycarbonyl;

R⁴⁷ is hydrogen, alkyl, hydroxyalkyl, alkoxyalkyl, cycloalkyl, cycloalkylalkyl, aryl, aralkyl, heteroaryl, heteroaralkyl, heterocycloalkyl, or heterocycloalkylalkyl; and

R⁴⁸ is hydrogen, alkyl, hydroxyalkyl, alkoxyalkyl, alkoxycarbonyl, aryloxycarbonyl, aralkyloxycarbonyl, heteroaryloxycarbonyl, heteroaralkyloxycarbonyl, alkylsulfonyl, arylsulfonyl, heteroarylsulfonyl, cycloalkyl, cycloalkylalkyl, aryl, aralkyl, heteroaryl, heteroaralkyl, heterocycloalkyl, or heterocycloalkylalkyl provided that one of R⁴⁷ and R⁴⁸ is other than hydrogen and wherein the aromatic or alicyclic rings in R⁴⁷ and R⁴⁸ are optionally substituted by one, two, or three R^{q} independently selected from alkyl, halo, hydroxy, hydroxyalkyl, alkoxy, alkoxyalkyl, haloalkyl, haloalkoxy, oxo, cyano, nitro, acyl, acyloxy, carboxy, alkoxycarbonyl, carbamoyl, alkylthio, alkylsulfinyl, alkylsulfonyl, alkoxycarbonylamino, alkylcarbamoyloxy, alkylsulfonylamino, aminosulfonyl, alkylaminosulfonyl, dialkylaminosulfonyl, amino, monosubsituted or disubstituted amino and one R^{q} selected from aryl, aralkyl, heteroaryl, heteroaralkyl, cycloalkyl, cycloalkylalkyl, heterocycloalkyl, heterocycloalkylalkyl, aryloxy, benzyloxy, aryloxycarbonyl, arylthio, arylsulfonyl, arylsulfinyl, aryloxycarbonylamino, arylcarbamoyloxy, arylsulfonylamino, arylaminosulfonyl, or aralkylaminosulfonyl wherein the aromatic and alicyclic rings in R^{q} are optionally substituted with one, two, or three R^{r} wherein R^{r} is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, monosubstituted amino, dialkylamino, aryl, heteroaryl, cycloalkyl, carboxy, carboxamido, or alkoxycarbonyl; or

R^{3f} and R^{3g} are fluoro;

(e) 7-(2,2-dimethylpropyl)-6-thiophen-2-ylmethyl-7H-pyrrolo-[2,3-d]pyrimidine-2-carbonitrile;

(f) morpholine-4-carboxylic acid [(S)-1-(4-cyano-1-methylpiperidine-4-ylcarbamoyl)-4,4-dimethylhexyl]amide;

(g) morpholine-4-carboxylic acid [(S)-1-(4-cyano-1-propylpiperidine-4-ylcarbamoyl)-3,3,4,4-tetramethylpentyl]amide;

(h) morpholine-4-carboxylic acid [(S)-1-(4-cyano-1-propylpiperidine-4-ylcarbamoyl)-4,4-dimethylpentyl]amide;

(i) morpholine-4-carboxylic acid [(S)-1-(4-cyano-1-propylpiperidine-4-ylcarbamoyl)-4,4-dimethylhexyl]amide;

(j) morpholine-4-carboxylic acid [(R)-1-(4-cyano-1-methylpiperidine-4-ylcarbamoyl)-4,4-dimethylhexyl]amide;

(k) 5,5-dimethyl-2-(2-oxo-2H-benzo[e][1,3]oxazin-4-ylamino)heptanoic acid (4-cyano-1-propylpiperidin-4-yl)amide;

(l) 5,5-dimethyl-2-(2-oxo-2H-benzo[e][1,3]oxazin-4-ylamino)heptanoic acid (4-cyano-1-(3-morpholin-4-ylpropyl)piperidin-4-yl)amide;

(m) 5,5-dimethyl-2-(2-oxo-2H-benzo[e][1,3]oxazin-4-ylamino)heptanoic acid (4-cyano-1-(2-morpholin-4-ylethyl)piperidin-4-yl)amide;

(n) 5,5-dimethyl-2-(2-oxo-2H-benzo[e][1,3]oxazin-4-ylamino)heptanoic acid {4-cyano-1-[2-(2-methoxyethoxy)ethyl]piperidin-4-yl}amide;

(O) 5,5-dimethyl-2-(2-oxo-2H-benzo[e][1,3]oxazin-4-ylamino)heptanoic acid (4-cyano-1-methylpiperidin-4-yl)amide;

(p) 2-(7-fluoro-2-oxo-2H-benzo[e][1,3]oxazin-4-ylamino)-5,5-dimethylheptanoic acid (4-cyano-1-propylpiperidin-4-yl)amide;

(q) 2-(7-fluoro-2-oxo-2H-benzo[e][1,3]oxazin-4-ylamino)-5,5-dimethylhexanoic acid {4-cyano-1-(2-morpholin-4-ylethyl)piperidin-4-yl}amide; or

(r) 2-(7-fluoro-2-oxo-2H-benzo[e][1,3]oxazin-4-ylamino)-5,5-dimethylhexanoic acid {4-cyano-1-[2-(2-methoxyethoxy)ethyl]piperidin-4-yl}amide; or

a pharmaceutically acceptable salt thereof.

Compounds generically and specifically disclosed in U.S. Pat. Nos. 6,353,017B1, 6,525,052 B2, 6,395,897B1, 6,313,117 B1, 6,635,621, 6,617,426, 6,698,057, 6,608,030, 6,593,327, 6,583,155, 6,579,896, 6,576,630, 6,569,847, 6,531,612, 6,525,036, 6,506,733, 6,492,362, 6,476,026, 6,462,076, 6,455,502, 6,420,364, 6,395,897, 6,287,840, 6,117,870, 6,030,947, 6,004,933, 5,976,858, 5,776,718, and 6,635,633 B2, PCT applications publication Nos. WO 00/55144, 00/55126, 00/07145, 00/55125, 01/19796, 01/19816A1, 01/49288, 01/77073, 02/051983, 02/098850, 02/098406, 02/23784, 02/070519, 02/080920, 02/069901, 03/075836, 02/100849A2, 03/024924, 03/037892A1, 03/029,200A2, 03/024923, 03/041649, and 02/0204485A1, 04/084842, 04/084843 and 04/083182; PCT application No. US03/19990 and US applications publication Nos. 2002/0040019 A1, 2003/0069240 A1, 2003/0114437 A1, 2003/0186962, 2003/0119827 A1, and 2003/0087939 A1, 2003/0203900, 2003/0199506, 2003/0186962, 2003/0158406, 2003/0158256, 2003/0158231, 2003/0153508, 2003/0144234, 2003/0119827, 2003/0119788, 2003/0114437, 2003,0105099, 2003/0100550, 2003/0096796, 2003/0092634, 2003/0087939, 2003/0078419, 2003/0073672, 2003/0069240,2002/0164765, 2002/0147189, 2002/0137932, 2002/0115656, 2002/0091259, 2002/0086996, 2002/0064856, 2002/0058809, 2002/0055497, 2002/0052378, 2002/0040020, 2002/0040019, 2002/0016361, 2001/0041700, and 2001/0008901 can also be used to practice the present invention. The compounds disclosed in the above patents and patent applications are incorporated herein by reference in their entirety.

Preferably, the Cathepsin S inhibitor is selected from:
N-[1R-(benzooxazol-2-yl-2-hydroxy-1S-phenethylethylcarbamoyl)-2-benzylsulfonylethyl]-morpholine-4-carboxamide;
2S-acetylamino-N-(2-oxazol-2-yl-2-hydroxy-1S-phenethylethyl)-3-cyclohexylpropionamide;
2S-acetylamino-N-(2-oxazol-2-yl-2-hydroxy-1S-phenethyl)-3-cyclohexylpropionamide;
2S-acetylamino-N-[2-hydroxy-1S-phenethyl-2-(5-phenyloxazol-2-yl)ethyl]-3-cyclohexylpropionamide;
2S-acetylamino-N-(S-benzothiazol-2-ylcarbonyl-3-phenylpropyl)-3-cyclohexylpropionamide;
N-[1S-(1S-phenethyl-2-benzooxazol-2-yl-1-oxoethylcarbamoyl)-2-naphth-2-ylethyl]-piperidine-4-carboxamide;
2S-acetylamino-N-(1S-benzooxazol-2-ylcarbonyl-3-phenylpropyl)-3-cyclohexylpropionamide;
tert-butyl 1S-(1S-benzooxazol-2-ylcarbonyl-3-phenylpropylcarbamoyl)-2-cyclohexylethyl carbamate;
2S-acetylamino-N-[1S-(5-phenyloxazol-2-ylcarbonyl)-3-phenylpropyl]-3-cyclohexylpropionamide;
benzyl 1S-(benzooxazol-2-ylcarbonylmethylcarbamoyl)-3-methylbutylcarbamate;
benzyl 1S-(5-phenylbenzooxazol-2-ylcarbonylmethylcarbamoyl)-3-methylbutylcarbamate;
2S-acetylamino-N-(1S-oxazol-2-ylcarbonyl-3-phenylpropyl)-3-cyclohexylpropionamide;
2-acetylamino-N-(1S-benzooxazol-2-ylcarbonyl-3-phenylpropyl)-3-phenylpropionamide;
benzyl 1S-(1S-benzooxazol-2-ylcarbonyl)-3-phenylpropylcarbamoyl)-2-naphthalen-2-yl-ethylcarbamate;
2-acetylamino-N-(1S-benzooxazol-2-ylcarbonyl-3-phenylpropyl]-3-(2-fluorophenyl)-propionamide;
2S-acetylamino-N-(1S-benzooxazol-2-ylcarbonyl-3-phenylpropyl)-2-methyl-3-phenylpropionamide;
tert-butyl 1S-(1S-benzooxazol-2-ylcarbonyl-3-phenylpropylcarbamoyl)-3-phenylpropyl-carbamate;
2-acetylamino-N-(1S-benzooxazol-2-ylcarbonyl-3-phenylpropyl)-1,2,3,4-tetrahydroiso-quinoline-3S-carboxamide;
2S-acetylamino-N-(1S-benzooxazol-2-ylcarbonyl-3-phenylpropyl)-3-(2-chlorophenyl)-propionamide;
2-acetylamino-N-(1-benzooxazol-2-ylcarbonyl-3-phenylpropyl)-3-(2-trifluoromethylphenyl)propionamide;
benzyl 1S-(1S-benzooxazol-2-ylcarbonyl-3-phenylpropylsulfamoylmethyl)-3-methylbutylcarbamate;
N-{1S-[1S-(benzooxazol-2-ylcarbonyl)-3-phenylpropylsulfamoylmethyl]-3-methylbutyl}acetamide;
benzyl 1R-(1S-benzooxazol-2-ylcarbonyl-3-phenylpropylsulfamoylmethyl)-3-methylbutylcarbamate;
N-[1-(1-benzooxazol-2-ylcarbonyl-3-phenylpropylsulfamoylmethyl)-3-methylbutyl]-acetamide;
tert-butyl 1R-(2-benzooxazol-2-yl-2-hydroxy-1S-phenethylethylcarbamoyl)-2-(2-cyanobenzylsulfanyl)ethylcarbamate;
tert-butyl 1R-(2-benzooxazol-2-yl-2-hydroxy-1S-phenethylethylcarbamoyl)-2-benzylsulfanylethylcarbamate;
N-[1R-(2-benzooxazol-2-yl-2-hydroxy-1S-phenethylethylcarbamoyl)-2-(2-cyanobenzylsulfanyl)ethyl]isonicotinamide;
9H-fluoren-9-ylmethyl 1S-(2-benzooxazol-2-yl-2-hydroxy-1S-phenethylethylcarbamoyl)-2-cyclohexylethylcarbamate;
tert-butyl 4-[1R-(2-benzooxazol-2-yl-2-hydroxy-1S-phenethylethylcarbamoyl]-2-(2-cyano benzylsulfanyl)ethylcarbamoylpiperidine-1-carboxylate;
benzyl 4-[1S-(2-benzooxazol-2-yl-2-hydroxy-1S-phenethylethylcarbamoyl)-2-cyclohexyl-ethylcarbamoyl]piperidine-1-carboxylate;
N-[1R-(2-benzooxazol-2-yl-2-hydroxy-1S-phenethylethylcarbamoyl)-2-benzylsulfonylethyl]-tetrahydropyran-4-carboxamide;
N-[1R-(2-benzooxazol-2-yl-2-hydroxy-1S-phenethylethylcarbamoyl)-2-benzylsulfonylethyl]-nicotinamide;
N-[1R-(2-benzooxazol-2-yl-2-hydroxy-1S-phenethylethylcarbamoyl)-2-benzylsulfonylethyl]pyrazine-2-carboxamide;
4-[1S-(2-benzooxazol-2-yl-2-hydroxy-1S-phenethylethylcarbamoyl)-2-cyclohexylethylcarbamoyl]piperidine-1-carboxylate;
N-[1S-(2-benzooxazol-2-yl-2-hydroxy-1S-phenethylethylcarbamoyl)-2-cyclohexylethyl]-isonicotinamide;
tert-butyl 4-[1R-(2-benzooxazol-2-yl-2-hydroxy-1S-phenethylethylcarbamoyl]-2-(3-methylpyrid-2-ylmethylsulfonyl)ethylcarbamoyl]piperidine-1-carboxylate;
tetrahydropyran-4-yl 1R-(2-benzooxazol-2-yl-2-hydroxy-1S-phenethylethylcarbamoyl)-2-benzylsulfanylethylcarbamate;
N-[1S-(2-benzooxazol-2-yl-2-hydroxy-1S-pbenethylethylcarbamoyl)-2-cyclohexylethyl]tetrahydropyran-4-carboxamide;
N-[1S-(2-benzooxazol-2-yl-2-hydroxy-1S-phenethylethylcarbamoyl)-2-cyclohexylethyl]-6-hydroxynicotinamide;
tert-butyl 4-[1R-(2-benzooxazol-2-yl-2-hydroxy-1S-phenethylethylcarbamoyl]-2-(2-cyano benzylsulfonyl)ethylcarbamoylpiperidine-1-carboxylate;
N-[1R-(2-benzooxazol-2-1-2-hydroxy-1S-phenethylethylcarbamoyl)-2-(2-cyanobenzylsulfonyl)ethyl]isonicotinamide;
tert-butyl 1R-(2-benzooxazol-2-yl-2-hydroxy-1S-phenethylethylcarbamoyl)-2-benzylsulfonylethylcarbamate;
N-[1R-(2-benzooxazol-2-yl-2-hydroxy-1S-phenethylethylcarbamoyl)-2-pyridin-3-yl-methylsulfonylethyl]pyrazine-2-carboxamide;
N-(2-benzooxazol-2-yl-2-hydroxy-1S-phenethylethyl)-3-cyclohexyl-2S-(3-pyridin-3-yl-ureido)propionamide;
N-[1S-(2-benzooxazol-2-yl-2-hydroxy-1S-phenethylethylcarbamoyl)-2-cyclohexylethyl]morpholine-4-carboxamide;
tert-butyl 4-[1R-(1S-benzooxazol-2-ylcarbonyl-3-phenylpropylcarbamoyl)-2-(2-cyanobenzylsulfonyl)ethylcarbamoyl]piperidine-1-carboxylate;
N-[1R-(1S-benzooxazol-2-ylcarbonyl)-3-phenylpropylcarbamoyl)-2-benzylsulfonylethyl]tetrahydropyran-4-carboxamide;
N-[1R-(1S-benzooxazol-2-ylcarbonyl-3-phenylpropylcarbamoyl)-2-benzylsulfonylethyl]nicotinamide;
N-[1R-(1S-benzooxazol-2-ylcarbonyl-3-phenylpropylcarbamoyl)-2-benzylsulfonylethyl]pyrazine-2-carboxamide;
tert-butyl 4-[1S-(1S-benzooxazol-2-ylcarbonyl-3-phenylpropylcarbamoyl)-2-cyclohexylethylcarbamoyl]piperidine-1-carboxylate;
tert-butyl 4-[1S-(1-benzooxazol-2-ylcarbonyl-3-phenylpropylcarbamoyl)-2-(6-methylpyridin-2-ylmethylsulfonyl)ethylcarbamoyl]piperidine-1-carboxylate;
N-[1R-(1S-benzooxazol-2-ylcarbonyl-3-phenylpropylcarbamoyl)-2-(2-cyanobenzylsulfonyl)ethyl]isonicotinamide;
tetrahydropyran-4-yl 1R-(1S-benzooxazol-2-ylcarbonyl-3-phenylpropylcarbamoyl)-2-benzylsulfonylethylcarbamate;
benzyl 4-[1S-(1S-benzooxazol-2-ylcarbonyl-3-phenylpropylcarbamoyl)-2-cyclohexylethylcarbamoyl]piperidine-1-carboxylate;
N-(1S-benzooxazol-2-ylcarbonyl-3-phenylpropyl)-3-cyclohexyl-2S-(3-pyridin-3-yl-ureido)propionamide;
N-[1S-(1S-benzooxazol-2-ylcarbonyl-3-phenylpropylcarbamoyl)-2-cyclohexylethyl]-morpholine-4-carboxamide;
N-[1S-(1S-benzooxazol-2-ylcarbonyl-3-phenylpropylcarbamoyl)-2-cyclohexylethyl-isonicotinamide;
N-[1S-(1S-benzooxazol-2-ylcarbonyl-3-phenylpropylcarbamoyl)-2-cyclohexylethyl]-tetrahydropyran-4-carboxamide;
N-[1S-(1S-benzooxazol-2-ylcarbonyl-3-phenylpropylcarbamoyl)-2-cyclohexylethyl]-6-hydroxynicotinamide;
N-[1R-(1S-benzooxazol-2-ylcarbonyl)-3-phenylpropylcarbamoyl)-2-benzylsulfonylethyl]morholine-4-carboxamide;
N-[1R-(2-benzooxazol-2-yl-1,1-dimethyl-2-oxoethylcarbamoyl)-2-benzylsulfonylethyl]morpholine-4-carboxamide;
N-[1R-(1S-benzooxazol-2-ylcarbonylpentylcarbamoyl)-2-(3,5-dimethylisoxazol-4-yl-methylsulfonyl)ethyl]morpholine-4-carboxamide;
N-[1R-(1S-benzooxazol-2-ylcarbonylpentylcarbamoyl)-2-(3,5-dimethylisoxazol-4-yl-methylsulfonylethyl]isonicotinamide;
N-[1R-(1S-benzooxazol-2-ylcarbonyl-3-phenylpropylcarbamoyl)-2-(2-cyanobenzylsulfonyl)ethyl]piperidine-4-carboxamide;
N-[1S-(1S-benzooxazol-2-ylcarbonyl-3-phenylpropylcarbamoyl)-2-cyclohexylethyl]-piperidine-4-carboxamide hydrochloride;
N-[1R-(1S-benzooxazol-2-ylcarbonyl-3-phenylpropylcarbamoyl)-2-(6-methylpyrid-2-yl-methylsulfonyl)ethyl]piperidine-4-carboxamide;
N-(1S-benzooxazol-2-ylcarbonylbutyl)-2R-methylsulfonylamino-3-benzylsulfonylpropionamide;
methyl 1R-(1S-benzooxazol-2-ylcarbonylbutylcarbamoyl)-2-benzylsulfonylethylcarbamate;
N-[1R-(1S-benzooxazol-2-ylcarbonylbutylcarbamoyl)-2-benzylsulfonylethyl]morpholine-4-carboxamide;
2S-acetylamino-N-(2-benzooxazol-2-yl-1S-butyl-2-hydroxyethyl)-3-cyclohexylpropionamide;
2S-acetylamino-N-(1S-benzooxazol-2-ylcarbonylpentyl)-3-cyclohexylpropionamide;
tert-butyl 1S-[1-benzooxazol-2-ylcarbonyl)-3-phenylpropylcarbamoyl)-2-cyclohexylethyl]carbamate;
2S-acetylamino-N-(1-benzooxazol-2-ylcarbonyl)-3-phenylpropyl)-3-cyclohexylpropionamide;
2S-acetylamino-N-(1-benzooxazol-2-ylcarbonylcyclobutyl)-3-cyclohexylpropionamide;
2S-acetylamino-N-(1R-benzooxazol-2-ylcarbonyl-3-phenylpropyl)-3-cyclohexylpropionamide;
2S-acetylamino-N-(2-benzooxazol-2-yl-2-hydroxy-1R-phenylpropyl)-3-cyclohexylpropionamide;
N-[1S-(1S-benzooxazol-2-ylcarbonyl-3-phenylpropylcarbamoyl)-2-cyclohexylethyl]-3H-imidazole-4-carboxamide;
N-[1S-(2-benzooxazol-2-yl-2-hydroxy-1S-phenylethylethylcarbamoyl)-2-cyclohexylethyl]-3H-imidazole-4-carboxamide;
tert-butyl 1R-(1-benzooxazol-2-ylcarbonylcyclobutylcarbamoyl)-2-benzylsulfonylethylcarbamate;
N-[1R-(1S-benzooxazol-2-ylcarbonyl-3-phenylpropylcarbamoyl)-2-o-tolylmethyl sulfonylethyl]morpholine-4-carboxamide;
N-[1R-(1S-benzooxazol-2-ylcarbonyl-3-phenylpropylcarbamoyl)-2-(2-nitrobenzylsulfonyl)ethyl]morpholine-4-carboxamide;
N-[1R-(1S-benzooxazol-2-ylcarbonyl-3-phenylpropylcarbamoyl)-2-(2-chlorobenzylsulfonyl)ethyl]morpholine-4-carboxamide;
N-[1R-(1S-benzooxazol-2-ylcarbonylpentylcarbamoyl)-2-benzylsulfonylethyl]-morpholine-4-carboxamide;
N-[1R-(1S-benzooxazol-2-ylcarbonylpentylcarbamoyl)-2-o-tolylmethylsulfonyl ethyl]morpholine-4-carboxamide;
N-[1R-(1S-benzooxazol-2-ylcarbonylpentylcarbamoyl)-2-(2-nitrobenzylsulfonyl)-ethyl]morpholine-4-carboxamide;
N-[1R-(1S-benzooxazol-2-ylcarbonylpentylcarbamoyl)-2-(2-chlorobenzylsulfonyl)-ethyl]morpholine-4-carboxamide;
N-[1R-(2-benzooxazol-2-yl-1,1-dimethyl-2-oxoethylcarbamoyl)-2-o-tolylmethyl sulfonylethyl]morpholine-4-carboxamide;
N-[1R-(2-benzooxazol-2-yl-1,1-dimethyl-2-oxoethylcarbamoyl)-2-(2-chlorobenzylsulfonyl)ethyl]morpholine-4-carboxamide;
N-[1R-(2-benzooxazol-2-yl-1,1-dimethyl-2-oxoethylcabamoyl)-2-(2-nitrobenzylsulfonyl)ethyl]morpholine-4-carboxamide;
N-[1R-(1S-benzooxazol-2-ylcarbonyl-3-phenylpropylcarbamoyl)-2-pyridin-2-ylmethylsulfonylethyl]piperidine-4-carboxamide;
N-[1R-(1S-benzooxazol-2-ylcarbonylpentylcarbamoyl)-2-pyridin-2-ylmethylsulfonylethyl]morpholine-4-carboxamide;
N-[1R-(1R-benzooxazol-2-ylcarbonyl-3-phenylpropylcarbamoyl)-2-benzylsulfonylethyl]morpholine-4-carboxamide;
N-[1R-(1-benzooxazol-2-ylcarbonylcyclobutylcarbamoyl)-2-benzylsulfonylethyl]-morpholine-4-carboxamide;
benzyl 1S-(2-benzooxazol-2-yl-2-hydroxyethylcarbamoyl)-3-methylbutylcarbamate;
2S-acetylamino-N-(2-benzooxazol-2-yl-1S-methyl-2-oxoethyl)-3-cyclohexylpropionamide;
tert-butyl 1R-(1-benzooxazol-2-ylcarbonylcyclobutylcarbamoyl)-2-benzylsulfanylethyl-carbamate;
N-[1R-(1S-benzooxazol-2-ylcarbonyl-3-methylsulfonylpropylcarbamoyl)-2-benzyl-sulfonylethyl]morpholine-4-carboxamide;
N-[1-(1S-benzooxazol-2-ylcarbonylpentylcarbamoyl)-3-phenylsulfanylpropyl]-morpholine-4-carboxamide;
N-[1R-(1S-benzooxazol-2-ylcarbonylpentylcarbamoyl)-2-(2-trifluoromethylbenzylsulfonyl)ethyl]morpholine-4-carboxamide;
N[1R-(1S-benzooxazol-2-ylcarbonyl-3-phenylpropylcarbamoyl)-2-pyridin-2-ylmethylsulfonylethyl]morpholine-4-carboxamide;
N-[1R-(1S-benzooxazol-2-ylcarbonyl-3-methylsulfonylpropylcarbamoyl)-2-pyridin-2-yl-methylsulfonylethyl]morpholine-4-carboxamide;
2-[2-(1-benzooxazol-2-ylcarbonylpentylcarbamoyl)-2-morpholin-4-ylcarbonylamino)-ethanesulfonylmethyl]pyridine-1-oxide;
N-[3-phenylsulfonyl-1-(1S-benzooxazol-2-ylcarbonylpentylcarbamoyl)propyl]-morpholine-4-carboxamide;
N-[1R-(1S-benzooxazol-2-ylcarbonylpentylcarbamoyl)-2-(2-difluoromethoxybenzylsulfonyl)ethyl]morpholine-4-carboxyamide;
N-[1R-(1S-benzooxazol-2-ylcarbonylpentylcarbamoyl)-2-(2-difluoromethoxybenzylsulfonyl)ethyl]isonicotinamide;
N-[1R-(1S-benzooxazol-2-ylcarbonylbutylcarbamoyl)-2-pyridin-2-ylmethylsulfonyl)-ethyl]morpholine-4-carboxamide;
2-[2R-(1S-benzooxazol-2-ylcarbonylbutylcarbamoyl)-2-morpholin-4-ylcarbonylamino-ethylsulfonylmethyl]pyridine-1-oxide;
1R-(1S-benzooxazol-2-ylcarbonylpentylcarbamoyl)-2-(2-difluoromethoxybenzylsulfonyl)ethylcarbamate;
2R-[3,3-bis(2-methoxyethyl)ureido]-N-(1S-benzooxazol-2-ylcarbonylpentyl)-3-benzylsulfonylpropionamide;
N-[1R-(1S-benzooxazol-2-ylcarbonyl-3-phenylpropylcarbamoyl)-2-(6-methylpyrid-2-yl-methylsulfonyl)ethyl]isonicotinamide;
N-[1R-(1S-benzooxazol-2-ylcarbonyl-3-phenylpropylcarbamoyl)-2-(2-trifluoromethylbenzylsulfonyl)ethyl]tetrahydropyran-4-carboxamide;
N-[1R-(1S-benzooxazol-2-ylcarbonyl-3-phenylpropylcarbamoyl)-2-thien-3-ylmethylsulfonylethyl]isonicotinamide;
N-[1R-(1S-benzooxazol-2-ylcarbonyl-3-phenylpropylcarbamoyl)-2-(6-methylpyrid-2-yl-methylsulfonyl)ethyl]tetrahydropyran-4-carboxamide;
N-[1R-(1-benzooxazol-2-ylcarbonylcyclobutylcarbamoyl)-2-(2-trifluoromethylbenzylsulfonyl)ethyl]tetrahydropyran-4-carboxamide;
N-[1R-(1S-benzooxazol-2-ylcarbonyl-3-phenylpropylcarbamoyl)-2-pyridin-3-ylmethylsulfonylethyl]pyrazine-2-carboxamide;
N-[1-(1-benzooxazol-2-ylcarbonyl-3-phenylpropylcarbamoyl)-2-thien-3-ylmethylsulfonylethyl]piperidine-4-carboxamide;
N-[1S-(1S-benzooxazol-2-ylcarbonyl-3-phenylpropylcarbamoyl)-2-thien-3-ylmethylsulfonylethyl]azetidine-3-carboxamide;
N-[1R-(1S-benzooxazol-2-ylcarbonyl)butylcarbamoyl)-2-pyridin-3-ylmethylsulfonylethyl]morpholine-4-carboxamide;
N-1R-(1S-benzooxazol-2-ylcarbonyl-3-phenylpropylcarbamoyl)-2-benzylsulfonylethyl]-piperazine-1-carboxamide;
N-[1R-(1S-benzooxazol-2-ylcarbonyl-3-methylsulfonylpropylcarbamoyl)-2-(2-di fluoromethoxybenzylsulfonyl)ethyl]morpholine-4-carboxamide;
N-[1R-(1S-benzooxazol-2-ylcarbonyl-3-methylesulfonylpropylcarbamoyl)-2-(2-m ethoxybenzylsulfonyl)ethyl]morpholine-4-carboxamide;
N-[1R-(1S-benzooxazol-2-ylcarbonylpentylcarbamoyl)-2-benzylsulfonylethyl]piperazine-1-carboxamide;
N-(1S-benzooxazol-2-ylcarbonyl-3-methylsulfonylpropyl)-2R-methylsulfonylamino-3-benzylsulfonylpropionamide;
methyl 1R-(1S-benzooxazol-2-ylcarbonyl-3-phenylpropylcarbamoyl)-2-pyridin-2-ylmethyl-sulfonylethylcarbamate;
methyl 1R-(1S-benzooxazol-2-ylcarbonyl-3-methylsulfonylpropylcarbamoyl)-2-benzylsulfonylethylcarbamate;
N-(1S-benzooxazol-2-ylcarbonylpentyl)-2R-[3,3-di(2-methoxyethyl)ureido]-3-pyridin-2-yl-methylsulfonylpropionamide;
N-[1R-(1S-benzooxazol-2-ylcarbonylbutylcarbamoyl)-2-(2-methoxybenzylsulfonyl)-ethyl]morpholine-4-carboxamide;
N-(1S-benzooxazol-2-ylcarbonylbutylcarbamoyl)-2R-(3,3-dimethylureido)-3-(2-methoxybenzylsulfonyl)propionamide;
N-(1S-benzooxazol-2-ylcarbonylbutyl)-2-methylsulfonylamino-3-(2-methoxybenzylsulfonyl)propionamide;
benzyl 1S-(2-benzooxazol-2-yl-2-hydroxy-1S-phenethylethylsulfamoylmethyl)-3-methylbutylcarbamate;
N-[1S-(2-benzooxazol-2-yl-2-hydroxy-1S-phenethylethylsulfamoylmethyl)-3-methylbutyl]acetamide;
benzyl 1S(2-benzooxazol-2-yl-2-hydroxy-1S-phenethylethylsulfamoylmethyl)-3-methylbutylcarbamate;
N-[1R-(2-benzooxazol-2-yl-2-hydroxy-1S-phenethylethylsulfamoylmethyl)-3-methylbutyl]acetamide;
2S-acetylamino-N-(2-benzooxazol-2-yl-2-hydroxy-1S-phenethylethyl)-3-cyclohexylpropionamide;
tert-butyl 1S-(2-benzooxazol-2-yl-2-hydroxy-1S-phenethylethyl]-2-cyclohexylethyl)-carbamate;
2-acetylamino-N-2-benzooxazol-2-yl-1,1-dimethyl-2-oxoethyl)-3-cyclohexylpropionamide;
benzyl 1S-[2-(5-phenylbenzooxazol-2-yl)-2-hydroxyethylcarbamoyl]-3-methylbutylcarbamate;
N-(2-benzooxazol-2-yl-2-hydroxy-1S-phenyethylethyl)-2-bicyclo[2.2.1]hept-2-yl-acetamide;
N-(2-benzooxazol-2-yl-2-hydroxy-1S-phenethylethyl)-2-naphthalen-1-ylacetamide;
N-(2-benzooxazol-2-yl-2-hydroxy-1S-phenethylethyl)-3-phenylpropionamide;
methyl 2-[2S-(3-cyclohexylpropionylamino)-1-hydroxy-4-phenylbutyl]-4,5-dihydrooxazole-4S-carboxylate;
methyl 2-[2S-(3-cyclohexylpropionylamino)-1-hydroxy-4-phenylbutyl]oxazole-4-carboxylate;
N-(2-benzooxazol-2-yl-2-hydroxy-1S-phenethyl)-4-cyclohexylbutyramide;
methyl 2-[2S-(3-cyclohexylpropionylamino)-1-hydroxy-4-phenylbutyl]-4,5-dihydrooxazole-4R-carboxylate;
2S-acetylamino-N-(2-benzooxazol-2-yl-2-hydroxy-1S-phenethylethyl)-3-(2-trifluoromethylphenyl)propionamide;
methyl 1-(1-benzooxazol-2-ylcarbonyl-3-phenylpropylcarbamoyl)-2-cyclohexylethylcarbamate;
N-(1-benzooxazol-2-ylcarbonyl-3-phenylpropyl)-3-cyclohexyl-2-methylsulfonyl aminopropionamide;
benzyl 1-(1-benzooxazol-2-ylcarbonyl-3-phenylpropylsulfamoylmethyl)-2-methylbutyl carbamate;
N-[1R-(1S-benzooxazol-2-ylcarbonyl-3-phenylpropylcarbamoyl)-2-(6-methylpyrid-2-yl-methylsulfonyl)ethyl]thiophene-3-carboxamide;
N-[1R-(1S-benzooxazol-2-ylcarbonyl)-3-phenylpropylcarbamoyl)-2-(2-methylpyrid-3-yl-methylsulfonyl)ethyl]nicotinamide;
N-[1R-(1S-benzooxazol-2-ylcarbonyl-3-phenylpropylcarbamoyl)-2-(2-cyanobenzyl sulfonyl)ethyl]azetidine-3-carboxamide;
tert-butyl 1R-(1-benzooxazol-2-ylcarbonylcyclobutylcarbamoyl)-2-(2-difluoromethoxybenzylsulfonyl)ethylcarbamate;
tert-butyl 1R-(1S-benzooxazol-2-ylcarbonyl-3-phenylpropylcarbamoyl)-2-(4-trifluoro-methylpyrid-3-ylmethylsulfonyl)ethylcarbamate;
N-[1R-(1-benzooxazol-2-ylcarbonylcyclobutylcarbamoyl)-2-(2-difluoromethoxybenzylsulfonyl)ethylmorpholine-4-carboxamide;
N-[1R-(1S-benzooxazol-2-ylcarbonylpentylcarbamoyl)-2-pyridin-3-ylmethylsulfonylethyl]isonicotinamide;
methyl 1R-(1S-benzooxazol-2-ylcarbonylbutylcarbamoyl)-2-(2-methoxybenzylsulfonyl) ethylcarbamate;
N-[1R-(1S-benzooxazol-2-ylcarbonylpropylcarbamoyl)-2-benzylsulfonylethyl]-morpholine-4-carboxamide;
N-(1R-benzooxazol 2-ylcarbonylpropyl)-2-(3,3-dimethylureido)-3-(2-methoxybenzylsulfonyl)propionamide;
methyl 1R-(1S-benzooxazol-2-ylcarbonylpropylcarbamoyl)-2-(2-methoxybenzylsulfonyl ethyl)carbamate;
N-(1-benzooxazol-2-ylcarbonylpentyl)-2R-[3,3-bis(2-methoxyethyl)ureido]-3-pyridin-3-yl-methylsulfonylpropionamide;
N-(1S-benzooxazol-2-ylcarbonylpentyl)-2R-[3,3-bis(2-methoxyethyl)ureido]-3-(3,5-dimethylisoxazol-4-ylmethylsulfonyl)propionamide;
N-(1S-benzooxazol-2-ylcarbonylpropyl)-3-(3,5-dimethylisoxazol-4-ylmethylsulfonyl)-2R-methylsulfonylaminopropionamide;
methyl 1R-(1S-benzooxazol-2-ylcarbonylpropylcarbamoyl)-2-(3,5-dimethylisoxazol-4-yl-methylsulfonyl)ethylcarbamate;
N-[1R-(1-benzooxazol-2-ylcarbonylpentylcarbamoyl)-2-pyridin-2-ylmethylsulfonylethyl]isonicotinamide;
4-[1R-(1S-benzooxazol-2-ylcarbonylpentylcarbamoyl)-2-pyridin-2-ylmethylsulfonyl-ethylcarbamoyl]pyridine-1-oxide;
benzyl 1S-[2-(4,5-dihydrooxazol-2-yl)-2-hydroxy-1S-phenethylethylcarbamoyl]-3-methylbutylcarbamate;
benzyl 1S-[2-(1H-benzoimidazol-2-yl)-2-hydroxy-1S-phenyethylethylcarbamoyl]-3-methylbutylcarbamate;
benzyl 1S-[2-(4,5-dihydro-4S-phenyloxazol-2-yl)-2-hydroxy-1S-phenethylethylcarbamoyl]-3-methylbutylcarbamate;
benzyl 1S-(2-benzooxazol-2-yl-2-hydroxy-1S-phenethylethylcarbamoyl)-3-methylbutyl carbamate;
benzyl 1-[2-(4,5-dihydro-5-phenyloxazol-2-yl)-2-hydroxy-1-phenethylethylcarbamoyl]-3-methylbutylcarbamate;
benzyl 1-[2-(4,5-dihydro-4S-methyl-5S-phenyloxazol-2-yl)-2-hydroxy-1-phenyethylcarbamoyl]-3-methylbutylcarbamate;
benzyl 3-methyl-1-(2-hydroxy-2-naphtho[2,3-d]oxazol-2-yl-1-phenethylethylcarbamoyl}-butylcarbamate;
benzyl 1S-(2-benzooxazol-2-yl-2-hydroxy-1S-phenethylethylcarbamoyl)-2-methylpropylcarbamate;
benzyl 1S-(2-benzooxazol-2-yl-2-hydroxy-1S-phenethylethylcarbamoyl)-3-methylbutyl carbamate;
benzyl 1S-[2-(4,5-dihydro-4,4-dimethyloxazol-2-yl)-2-hydroxy-1S-phenethylethylcarbamoyl]-3-methylbutylcarbamate;
methyl 2-[2-(2-benzyloxycarbonylamino-4-methylvalerylamino)-1-hydroxy-4-phenylbutyl]-4,5-dihydrooxazole-4-carboxylate;
methyl 2-[2-(2,2-dimethylpropionylamino)-4-phenylbutyryl]oxazole-4-carboxylate;
tert-butyl 4-{1S-[2-(5-tert-butylbenzooxazol-2-yl)-2-hydroxy-1S-phenethylethylcarbamoyl]-3-methylbutylcarbamoyl}piperidine-1-carboxylate;
tert-butyl 4-{1S-[2-hydroxy-1S-phenethyl-2-(5-sulfamoylbenzooxazol-2-yl)ethylcarbamoyl]-3-methylbutylcarbamoyl}piperidine-1-carboxylate;
tert-butyl 4-[1S-(2-hydroxy-2-naphtho[1,2-d]oxazol-2-yl-1S-phenethylethylcarbamoyl)-3-methylbutylcarbamoyl]piperidine-1-carboxylate;
tert-butyl 4-[1S-(2-hydroxy-2-naphtho[2,1-d]oxazol-2-yl-1S-phenethylethylcarbamoyl)-3-methylbutylcarbamoyl]piperidine-1-carboxylate;
tert-butyl 4-{1S-[2-hydroxy-1S-phenethyl-2-(5-phenylbenzooxazol-2-yl)ethylcarbamoyl]-3-methylbutylcarbamoyl}piperidine-1-carboxylate;
tert-butyl 4-[1S-(2-benzooxazol-2-yl)-2-hydroxy-1S-phenethylethylcarbamoyl)-2-methylbutylcarbamoyl]piperidine-1-carboxylate;
tert-butyl 3-[1S-(2-benzooxazol-2-yl)-2-hydroxy-1S-phenethylethylcarbamoyl)-2-methylbutylcarbamoyl]benzylcarbamate;
tert-butyl 4-[1S-(2-benzooxazol-2-yl)-2-hydroxy-1S-phenethylethylcarbamoyl)-2-cyclohexylethylcarbamoyl]piperidine-1-carboxylate;
benzyl 3-methyl-1S-[2-hydroxy-1S-phenethyl-2-(5-phenyloxazol-2-yl)ethylcarbamoyl]butylcarbamate;
pyridin-3-yl 3-methyl-1S-[2-hydroxy-1S-phenethyl-2-(5-phenyloxazol-2-yl)ethylcarbamoyl]butylcarbamate;
benzyl 1S-[2-hydroxy-1S-phenethyl-2-(5-phenyloxazol-2-yl)ethylsulfamoylmethyl]-2R-methylbutylcarbamate;
benzyl 3-methyl-1S-(1S-pyridin-2-ylcarbonyl-3-phenylpropylcarbamoyl)butylcarbamate;
benzyl 1-[1-(pyridin-3-ylcarbonyl)-3-phenylpropylcarbamoyl]-3-methylbutylcarbamate;
benzyl 1-[1-(quinolin-3-ylcarbonyl)-3-phenylpropylcarbamoyl]-3-methylbutylcarbamate;
benzyl 1-[1-(1H-indol-5-ylcarbonyl)-3-phenylpropylcarbamoyl]-3-methylbutylcarbamate;
benzyl 1-[1-(benzofuran-2-ylcarbonyl)-3-phenylpropylcarbamoyl]-3-methytbutylcarbamate;
benzyl 1-[1-(benzothiazol-2-ylcarbonyl)-3-phenylpropylcarbamoyl]-3-methylbutylcarbamate;
benzyl 3-methyl-1S-(3-phenyl-1S-thiazol-2-ylcarbonylpropylcarbamoyl)butylcarbamate;
N-[3-methyl-1S-(3-phenyl-1S-thiazol-2-ylcarbonylpropylcarbamoyl)butyl]-4-methyl-piperazine-1-carboxamide;
tert-butyl 4-[3-methyl-1S-(3-phenyl-1S-thiazol-2-ylcarbonylpropylcarbamoyl)butylcarbamoyl]piperazine-1-carboxylate;
benzyl 3-methyl-1S-(3-phenyl-1S-thien-2-ylcarbonylpropylcarbamoyl)butylcarbamate;
benzyl 1S-[1S-(1-methyl-1H-imidazol-2-ylcarbonyl-3-phenylpropylcarbamoyl]-methylbutylcarbamate;
benzyl 1S-(1S-thiazol-2-ylcarbonyl-3-phenylpropylcarbamoyl)-2-methylpropylcarbamate;
N-[3-methyl-1S-(3-phenyl-1S-thiazol-2-ylcarbonylpropylcarbamoyl)butyl]piperazine-1-carboxamide;
benzyl 1S-[1S-(4-methylthiazol-2-ylcarbonyl)-3-phenylpropylcarbamoyl]-3-methylbutylcarbamate;
benzyl 1S-(1S-furan-2-ylcarbonyl-3-phenylpropylcarbamoyl)-3-methylbutylcarbamate;
benzyl 1S-[1S-(1-benzyl-1H-imidazol-2-ylcarbonyl-3-phenylpropylcarbamoyl]-3-methylbutylcarbamate;
benzyl 3-phenyl-1-(4,5-dihydro-4S-phenyloxazol-2-ylcarbonyl)propyl]carbamate;
benzyl 3-phenyl-1-(4,5-dihydro-5-phenyloxazol-2-ylcarbonylpropyl]carbamate;
benzyl [1-(4,5-dihydro-4S-methyl-5S-phenyloxazol-2-ylcarbonyl)-3-phenylpropyl]-carbamate;
ethyl 2-[2-(2-benzyloxycarbonylamino-4-methylvalerylamino)-4-phenylbutyryl]thiazole-4-carboxylate;
methyl 2-[2-(2-benzyloxycarbonylamino-4-methylvalerylamino)-1-hydroxy-4-phenylbutyl]oxazole-4-carboxylate;
2-[2-(2-benzyloxycarbonylamino-4-methylvalerylamino)-1-hydroxy-4-phenylbutyl]-oxazole-4-carboxylic acid;
benzyl 3-methyl-1-[2-hydroxy-1-phenethyl-2-(4-phenylcarbamoyloxazol-2-yl)ethylcarbamoyl]butylcarbamate;
benzyl 1-[2-(4-benzylcarbamoyloxazol-2-yl)-2-hydroxy-1-phenethylethylcarbamoyl]-3-methylbutylcarbamate;
benzyl 3-methyl-1-[2-hydroxy-1-phenethyl-2-(4-phenyethylcarbamoyloxazol-2-yl)ethylcarbamoyl]butylcarbamate;
benzyl 1-[1-(4,5-dihydro-4S-phenyloxazol-2-ylcarbonyl)-3-phenylpropylcarbamoyl]-3-methylbutylcarbamate;
benzyl 1S-(1S-benzooxazol-2-ylcarbonyl-3-pentylcarbamoyl)-3-methylbutylcarbamate;
benzyl 1S-[1S-(4,5-dihydrooxazol-2-ylcarbonyl)-3-phenylpropylcarbamoyl]-3-methylbutylcarbamate;
N-[3-methyl-1S-(3-phenyl-1S-benzooxazol-2-ylcarbonylpropylcarbamoyl)butyl]-piperidine-4-carboxamide;
benzyl 1-[1-(4,5-dihydro-5-phenyloxazol-2-ylcarbonyl)-3-phenylpropylcarbamoyl]-3-methylbutylcarbamate;
benzyl 1-[1-(4,5-dihydro-5S-phenyl-4S-methyloxazol-2-ylcarbonyl)-3-phenylpropylcarbamoyl)-3-methylbutylcarbamate;
benzyl 1S-(1S-phenethyl-2-benzimidazol-2-yl-1-oxoethylcarbamoyl)-3-methylbutylcarbamate;
benzyl 1-[1-(naphtho[2,3-d]oxazol-2-ylcarbonyl)-3-phenylpropylcarbamoyl]-3-methyl butylcarbamate;
methyl 2-[2-(2-benzyloxycarbonylamino-4-methylvalerylamino)-4-phenylbutyryl]4,5-dihydrooxazole-4-carboxylate;
benzyl 1S-[1S-(4,5-dihydro-4,4-dimethyloxazol-2-ylcarbonyl)-3-phenylpropyl-carbamoyl]-3-methylbutylcarbamate;
benzyl 1S-(1S-benzooxazol-2-ylcarbonyl-3-phenylpropylcarbamoyl)-2-methylpropylcarbamate;
benzyl 1S-(1S-benzooxazol-2-ylcarbonyl-3-phenylpropylcarbamoyl)-2-methylbutylcarbamate;
benzyl 1S-[1S-(5-chlorobenzooxazol-2-ylcarbonyl)-3-phenylpropylcarbamol]-3-methylbutylcarbamate;
N-{3-methyl-1S-[3-phenyl-1S-(5-chlorobenzooxazol-2-ylcarbonyl)propylcarbamoyl]-butyl}piperidine-4-carboxamide;
N-[2-cyclohexyl-1S-(3-phenyl-1S-benzooxazol-2-ylcarbonylpropylcarbamoyl)ethyl]-piperidine-4-carboxamide;
methyl 2-(2-benzyloxycarbonylamino-4-methylvalerylamino)-4-phenylbutyryloxazole-4-carboxylate;
benzyl 1-[1-(4-phenylcarbamoyloxazol-2-ylcarbonyl)-3-phenylpropylcarbamoyl]-3-methylbutylcarbamate;
benzyl 1-[1-(4-benzylcarbamoyloxazol-2-ylcarbonyl)-3-phenylproylcarbamoyl]-3-methylbutylcarbamate;
tert-butyl 4 {1S-[1S-(5-tert-butylbenzooxazol-2-ylcarbonyl)-3-phenylpropylcarbamoyl]-3-methylbutylcarbamoyl}piperidine-1-carboxylate;
tert-butyl 4-{1S-[1S-(5-sulfamoylbenzooxazol-2-ylcarbonyl)-3-phenypropylcarbamoyl]-3-methylbutylcarbamoyl}piperidine-1-carboxylate;
N-{3-methyl-1S-[3-phenyl-1S-(5-tert-butylbenzooxazol-2-ylcarbonyl)propylcarbamoyl]butyl}piperidine-4-carboxamide;
N-{3-methyl-1S-[3-phenyl-1S-(5-sulfamoylbenzooxazol-2-ylcarbonyl)propylcarbamoyl]-butyl}piperidine-4-carboxamide;
tert-butyl 4-[1S-(1S-naphtho[1,2-d]oxazol-2-ylcarbonyl-3-phenylpropylcarbamoyl)-3-methylbutylcarbamoyl]piperidine-1-carboxylate;
tert-butyl 4-[1S-(1S-naphtho[2,1-d]oxazol-2-ylcarbonyl-3-phenylpropylcarbamoyl)-3-methylbutylcarbamoyl]piperidine-1-carboxylate;
tert-butyl 4-{1S-[1S-(5-phenylbenzooxazol-2-ylcarbonyl)-3-phenylproylcarbamoyl]-3-methylbutylcarbamoyl}piperidine-1-carboxylate;
N-{3-methyl-1S-[3-phenyl-1S-(naphtho[1,2-d]oxazol-2-ylcarbonyl)propylcarbamoyl]butyl}piperidine-4-carboxamide;
N-{3-methyl-1S-[3-phenyl-1S-(naphtho[2,1-d]benzooxazol-2-ylcarbonyl)propylcarbamoyl]butyl}piperidine-4-carboxamide;
N-{3-methyl-1-[3-phenyl-1-(5-phenylbenzooxazol-2-ylcarbonyl)propylcarbamoyl]-butyl}piperidine-4-carboxamide;
benzyl 1-[1-(4-phenyethylcarbamoyloxazol-2-ylcarbonyl)-3-phenylpropylcarbamoyl]-3-methylbutylcarbamate;
benzyl 1-{1-[4-(3-phenylpropylcarbamoyl)oxazol-2-ylcarbonyl]-3-phenylpropylcarbamoyl}-3-methylbutylcarbamate;
tert-butyl 4-[1S-(1S-benzooxazol-2-ylcarbonyl-3-phenylpropylcarbamoyl)-2-methylbutylcarbamoyl]piperidine-1-carboxylate;
tert-butyl 3-[1S-(1S-benzooxazol-2-ylcarbonyl-3-phenylpropylcarbamoyl)-2-methylbutylcarbamoyl]benzylcarbamate;
N-[2-methyl-1S-(3-phenyl-1S-benzooxazol-2-ylcarbonylpropylcarbamoyl)butyl]-piperidine-4-carboxamide;
N-[2-methyl-1S-(3-phenyl-1S-benzooxazol-2-ylcarbonylpropylcarbamoyl)butyl]-3-aminomethylbenzamide;
benzyl 1-{1-[4-(2-indol-3-ylethylcarbamoyl)oxazol-2-ylcarbonyl]-3-phenylpropylcarbamoyl}-3-methylbutylcarbamate;
benzyl 1-[1-(4-methylcarbamoyloxazol-2-ylcarbonyl)-3-phenylpropylcarbamoyl]-3-methylbutylcarbamate;
benzyl 2-{2-[2-(2-benzyloxycarbonylamino-4-methylvalerylamino)-4-phenylbutyryl]-oxazol-2-ylcarbonylamino}valerate;
benzyl 1S-{1S-[4-(4-benzylpiperidin-1-ylcarbonyl)oxazol-2-ylcarbonyl]-3-phenylpropylcarbamoyl}-3-methylbutylcarbamate;
benzyl 1S-[1S-(4-furan-2-ylmethylcarbamoyloxazol-2-ylcarbonyl)-3-phenylpropylcarbamoyl]-3-methylbutylcarbamate;
benzyl 3-methyl-1S-[1S-(4-pyridin-2-ylmethylcarbamoyloxazol-2-ylcarbonyl)-3-phenylpropylcarbamoyl]butylcarbamate;
benzyl 3-methyl-1S-[1S-(4-pyridin-3-ylmethylcarbamoyloxazol-2-ylcarbonyl)-3-phenylpropylcarbamoyl]butylcarbamate;
benzyl 3-methyl-1S-[1S-(4-pyridin-4ylmethylcarbamoyloxazol-2-ylcarbonyl)-3-phenylpropylcarbamoyl]butylcarbamate;
benzyl 1S-{1S-[4-(2-chlorobenzylcarbamoyl)oxazol-2-ylcarbonyl]-3-phenylpropylcarbamoyl}-3-methylbutylcarbamate;
benzyl 1S-{1S-[4-(3-chlorobenzylcarbamoyl)oxazol-2-ylcarbonyl]-3-phenylpropylcarbamoyl}-3-methylbutylcarbamate;
benzyl 1S-{1S-[4-(4-chlorobenzylcarbamoyl)oxazol-2-ylcarbonyl]-3-phenylpropylcarbamoyl}-3-methylbutylcarbamate;
benzyl 3-methyl-1S-[1S-(4-diphenylmethylmethylcarbamoyloxazol-2-ylcarbonyl)-3-phenylpropylcarbamoyl]-3-methylbutylcarbamate;
benzyl 1S-[1S-(4-adamantan-1-ylmethylcarbamoyloxazol-2-ylcarbonyl)-3-phenylpropyl carbamoyl]-3-methylbutylcarbamate;
benzyl 1-{1-[4-(1-methylethylcarbamoyl)oxazol-2-ylcarbonyl]-3-phenylpropylcarbamoyl}-3-methylbutylcarbamate;
benzyl 1-{1-[4-(1S-phenylethylcarbamoyl)oxazol-2-ylcarbonyl]-3-phenylpropylcarbamoyl}-3-methylbutylcarbamate;
benzyl 1-{1-[4-(1R-phenylethylcarbamoyl)oxazol-2-ylcarbonyl]-3-phenylpropylcarbamoyl}-3-methylbutylcarbamate;
benzyl 1-{1-[4-(N-benzyl-N-methylcarbamoyl)oxazol-2-ylcarbonyl]-3-phenylpropylcarbamoyl}-3-methylbutylcarbamate;
benzyl 1-[1-(4-pyrrolidin-1-ylcarbonyloxazol-2-ylcarbonyl)-3-phenylpropylcarbamoyl]-3-methylbutylcarbamate;
benzyl 1-[1-(4-piperidin-1-ylcarbonyloxazol-2-ylcarbonyl)-3-phenylpropylcarbamoyl]-3-methylbutylcarbamate;
benzyl 1-{1-[4-(2,3-dihydroindol-1-ylcarbonyl)oxazol-2-ylcarbonyl]-3-phenylpropyl carbamoyl}-3-methylbutylcarbamate;
benzyl 1-{1-[4-(3,4-dihydro-1H-isoquinolin-2-ylcarbonyl)oxazol-2-ylcarbonyl]-3-phenylpropylcarbamoyl}-3-methyl;
benzyl 1-{1-[4-(3,4-dihydro-2H-quinolin-1-ylcarbonyl)oxazol-2-ylcarbonyl]-3-phenylpropylcarbamoyl}-3-methylbutylcarbamate;
benzyl 1-[1-(4-naphth-1-ylmethylcarbamoyloxazol-2-ylcarbonyl)-3-phenylpropylcarbamoyl]-3-methylbutylcarbamate;
tert-butyl 4-[1S-(1S-benzooxazol-2-ylcarbonyl)-3-phenylpropylcarbamoyl)-2-cyclohexylethylcarbamoyl]piperidine-1-carboxylate;
1S-{1S-[4-(3,4-dihydro-2H-quinol-1-ylcarbonyl)oxazol-2-ylcarbonyl]-ethylcarbamoyl}-3-methylbutylcarbamate;
benzyl 3-methyl-1S-[1S-(5-phenyloxazol-2-ylcarbonyl)-3-phenylpropylcarbamoyl]-butylcarbamate;
pyridin-3-yl 3-methyl-1S-[1S-(5-phenyloxazol-2-ylcarbonyl)-3-phenylpropylcarbamoyl]-butylcarbamate;
benzyl 1S-[1s-(5-phenyloxazol-2-ylcarbonyl)-3-phenylpropylsulfamoylmethyl]-2R-methylbutylcarbamate;
benzyl 3-methyl-1-{2-hydroxy-1-phenethyl-2-[4-(3-phenylpropylcarbamoyl)oxazol-2-yl]-ethylcarbamoyl}butylcarbamate;
benzyl 1-{2-hydroxy-2-[4-(2-indol-3-ylethylcarbamoyl)oxazol-2-yl]-phenethylethylcarbamoyl}-3-methylbutylcarbamate;
benzyl 3-methyl-1-[2-hydroxy-2-(4-methylcarbamoyloxazol-2-yl)-1-phenethylethylcarbamoyl]butylcarbamate;
benzyl 2-{2-[2-(2-benzyloxycarbonylamino-4-methylvalerylanino)-1-hydroxy-4-phenyl butyl]oxazol-2-ylcarbonylamino}valerate;
benzyl 1S-{2-[4-(4-benzylpiperidin-1-ylcarbonyl)oxazol-2-yl]-2-hydroxy-1S-phenethylethylcarbamoyl}-3-methylbutylcarbamate;
benzyl 1S-[2-(4-furan-2-ylmethylcarbamoyloxazol-2-yl)-2-hydroxy-1S-phenethylethylcarbamoyl]-3-methylbutylcarbamate;
benzyl 3-methyl-1S-[2-hydroxy-1S-phenethyl-2-(4-pyridin-2-ylmethylcarbamoyloxazol-2-yl)ethylcarbamoyl]butylcarbamate;
benzyl 3-methyl-1S-[2-hydroxy-1S-phenethyl-2-(4-pyridin-3-ylmethylcarbamoyloxazol-2-yl)ethylcarbamoyl]butylcarbamate;
benzyl 3-methyl-1S-[2-hydroxy-1S-phenethyl-2-(4-pyridin-4-ylmethylcarbamoyloxazol-2-yl)ethylcarbamoyl]butylcarbamate;
benzyl 3-methyl-1S-{2-[4-(2-chlorobenzylcarbamoyl)oxazol-2-yl]-2-hydroxy-1S-phenethylethylcarbamoyl}butylcarbamate;
benzyl 3-methyl-1S-{2-[4-(3-chlorobenzylcarbamoyl)oxazol-2-yl]-2-hydroxy-1S-phenethylethylcarbamoyl}butylcarbamate;
benzyl 3-methyl-1S-{2-[4-(4-chlorobenzylcarbamoyl)oxazol-2-yl]-2-hydroxy-1S-phenethylethylcarbamoyl}butylcarbamate;
benzyl 3-methyl-1S-{2-hydroxy-1S-phenethyl-2-[4-(2R-phenylcycloprop-1S-yl-carbamoyl)oxazol-2-yl]ethylcarbamoyl}butylcarbamate;
benzyl 1S-[2-(4-adamantan-1-ylmethylcarbamoyloxazol-2-yl)-2-hydroxy-methyl)-1S-phenethylethylcarbamoyl]-3-methylbutylcarbamate;
benzyl 3-methyl-1S-[2-hydroxy-1S-phenethyl-2-(4-diphenylmethylcarbamoyloxazol-2-yl)ethylcarbamoyl]butylcarbamate;
benzyl 3-methyl-1-{2-hydroxy-2-[4-(1-methylethylcarbamoyl)oxazol-2-yl]-1-phenethylethylcarbamoyl}butylcarbamate;
benzyl 3-methyl-1-{2-hydroxy-1-phenethyl-2-[4-(2S-phenyethylcarbamoyl)oxazol-2-yl]ethylcarbamoyl}butylcarbamate;
benzyl 3-methyl-1-{2-hydroxy-1-phenethyl-2-[4-(2R-phenyethylcarbamoyl)oxazol-2-yl]ethylcarbamoyl}butylcarbamate;
benzyl 3-methyl-1-{2-[4-(N-benzyl-N-methylcarbamoyl)oxazol-2-yl]-2-hydroxy-1-phenethylethylcarbamoyl}butylcarbamate;
benzyl 3-methyl-1-[2-hydroxy-1-phenethyl-2-(4-pyrrolidin-1-ylcarbonyloxazol-2-yl) ethylcarbamoyl]butylcarbamate;
benzyl 3-methyl-1-[2-hydroxy-1-phenethyl-2-(4-piperidin-1-ylcarbonyloxazol-2-yl)ethylcarbamoyl]butylcarbamate;
benzyl 3-methyl-1-{2-[4-(2,3-dihydroindol-1-ylcarbonyl)oxazol-2-yl]-2-hydroxy-1-phenethylethylcarbamoyl}butylcarbamate;
benzyl 3-methyl-1-{2-[4-(3,4-dihydro-1H-isoquinolin-2-ylcarbonyl)oxazol-2-yl]-2-hydroxy-1-phenethylethylcarbamoyl}butylcarbamate;
benzyl 3-methyl-1-{2-[4-(3,4-dihydro-1H-isoquinolin-1-ylcarbonyl)oxazol-2-yl]-2-hydroxy-1-phenethylethylcarbamoyl}butylcarbamate;
benzyl 3-methyl-1-[2-hydroxy-2-(4-naphth-1-ylmethylcarbonyloxazol-2-yl)-1-phenethylethylcarbamoyl]butylcarbamate;
benzyl 1S-{2-[4-(3,4-dihydro-2H-quinol-1-ylcarbonyl)oxazol-2-yl]-2-hydroxy-1S-methylethlcarbamoyl}-3-methylbutylcarbamate;
N-[3-methyl-1S-(1S-thiazol-2-ylcarbonylethylcarbamoyl)butyl]-4-morpholin-4-yl-benzamide;
N-[1S-(2-benzooxazol-2-yl-1,1-dimethyl-2-oxoethylcarbamoyl)-3-methylbutyl]-4-(4-methylpiperazin-1-yl)benzamide;
N-[1R-(1S-benzooxazol-2-yl-carbonylprop-1-ylcarbamoyl)-2-(2-methylprop-1-yl-sulfonyl)ethyl]morpholine-4-carboxamide;
N-[1R-(1S-benzooxazol-2-yl-carbonylprop-1-ylcarbamoyl)-2-cyclopropylmethylsulfonylethyl]morpholine-4-carboxamide;
N-{(R)-1-(cyanomethylcarbamoyl)-2-[2-(1,1-difluoromethoxy)-phenylmethanesulfonyl]-ethyl}-nicotinamide;
N-{(R)-1-(cyanomethylcarbamoyl)-2-[2-(1,1-difluoromethoxy)-phenylmethanesulfonyl]-ethyl}-isonicotinamide;
pyridine-2-carboxylic acid-{(R)-1-(cyanomethylcarbamoyl)-2-[2-(1,1-difluoromethoxy)-phenylmethanesulfonyl]-ethyl}-amide;
pyrazine-2-carboxylic acid-{(R)-1-(cyanomethylcarbamoyl)-2-[2-(1,1-difluoromethoxy)-phenylmethanesulfonyl]-ethyl}-amide;
N-{(R)-1-(cyanomethyl-carbamoyl)-2-[2-(1,1-difluoromethoxy)-phenylmethanesulfonyl]-ethyl}-6-hydroxynicotinamide;
2-amino-N-{(R)-1-(cyanomethylcarbamoyl)-2-[2-(1,1-difluoromethoxy)-phenylmethanesulfonyl]ethyl}-nicotinamide;
6-amino-N-{(R)-1-(cyanomethylcarbamoyl)-2-[2-(1,1-difluoromethoxy)-phenylmethanesulfonyl]-ethyl}-nicotinamide;
3-hydroxypyridine-2-carboxylic acid-{(R)-1-(cyanomethylcarbamoyl)-2-[2-(1,1-difluoromethoxy)phenylmethanesulfonyl]ethyl}amide;
morpholine-4-carboxylic acid-{(R)-1-(4-cyanotetrahydropyran-4-ylcarbamoyl)-2-[2-(1,1-difluoromethoxy)phenylmethanesulfonyl]ethyl}amide;
morpholine-4-carboxylic acid-{(R)-1-(4-cyano-1-methylpiperidin-4-ylcarbamoyl)-2-[2-(1,1-difluoromethoxy)phenylmethanesulfonyl]ethyl}amide;
(R)—N-cyanomethyl-3-[2-(1,1-difluoro-methoxy)-phenylmethanesulfonyl]-2-(3,3-dimethylureido)propionamide;
{(R)-1-(cyanomethylcarbamoyl)-2-[2-(1,1-difluoromethoxy)phenylmethanesulfonyl]-ethyl}carbamic acid allyl ester;
5 {(R)-1-(cyanomethylcarbamoyl)-2-[2-(1,1-difluoromethoxy)phenylmethanesulfonyl]-ethyl}-carbamic acid isopropyl ester;
{(R)-1-(cyanomethylcarbamoyl)-2-[2-(1,1-difluoromethoxy)phenylmethanesulfonyl]-ethyl}-carbamic acid isobutyl ester;
N-{(R)-1-(cyanomethylcarbamoyl)-2-[2-(1,1-difluoromethoxy)phenylmethanesulfonyl]-ethyl}-3,4-difluorobenzamide;
N-{(R)-1-(cyanomethylcarbamoyl)-2-[2-(1,1-difluoromethoxy)phenylmethanesulfonyl]-ethyl}-3-methylbenzamide;
thiophene-2-carboxylic acid-{(R)-1-(cyanomethylcarbamoyl)-2-[2-(1,1-difluoromethoxy)-phenylmethanesulfonyl]ethyl}amide;
4-bromo-N-{(R)-1-(cyanomethylcarbamoyl)-2-[2-(1,1-difluoromethoxy)-phenylmethanesulfonyl]ethyl}benzamide;
N-{(R)-1-(cyanomethylcarbamoyl)-2-[2-(1,1-difluoromethoxy)phenylmethanesulfonyl]-ethyl}-4-methoxy-benzamide;
N-{(R)-1-(cyanomethylcarbamoyl)-2-[2-(1,1-difluoromethoxy)phenylmethanesulfonyl]-ethyl}-4-trifluoromethoxybenzamide;
naphthalene-2-carboxylic acid-{(R)-1-(cyanomethylcarbamoyl)-2-[2-(1,1 difluoromethoxy)phenylmethanesulfonyl]ethyl}amide;
(E)-N-{(R)-1-(cyanomethyl-carbamoyl)-2-[2-(1,1-difluoromethoxy)-phenylmethanesulfonyl]ethyl}-3-phenylacrylamide;
5-methyl-thiophene-2-carboxylic acid-{(R)-1-(cyanomethyl-carbarnoyl)-2-[2-(1,1-difluoromethoxy)phenylmethanesulfonyl]ethyl}amide;
biphenyl-4-carboxylic acid-{(R)-1-(cyanomethyl-carbamoyl)-2-[2-(1,1-difluoromethoxy)-phenylmethanesulfonyl]ethyl}amide;
1H-indole-5-carboxylic acid-{(R)-1-(cyanomethylcarbamoyl)-2-[2-(1,1-difluoromethoxy)-phenylmethanesulfonyl]ethyl}amide;
benzo[1,3]dioxole-5-carboxylic acid-{(R)-1-(cyanomethylcarbamoyl)-2-[2-(1,1-difluoromethoxy)phenylmethanesulfonyl]ethyl}amide;
benzo[b]thiophene-2-carboxylic acid-{(R)-1-(cyanomethylcarbamoyl)-2-[2-(1,1-difluoromethoxy)-phenylmethanesulfonyl]ethyl}-amide;
N-{(R)-1-(cyanomethylcarbamoyl)-2-[2-(1,1-difluoromethoxy)-phenylmethanesulfonyl]-ethyl)-3-phenoxybenzamide;
quinoline-3-carboxylic acid-{(R)-1-(cyanomethylcarbamoyl)-2-[2-(1,1-difluoromethoxy)-phenylmethanesulfonyl]ethyl}amide;
N-{(R)-1-(cyanomethylcarbamoyl)-2-[2-(1,1-difluoromethoxy)phenylmethanesulfonyl)-ethyl}-3-(1-phenylmethanoyl)benzamide;
4-chloro-N-{(R)-1-(cyanomethylcarbamoyl)-2-[2-(1,1-difluoromethoxy)-phenylmethanesulfonyl]-ethyl}-benzamide;
N-{(R)-1-(cyanomethylcarbamoyl)-2-[2-(1,1-difluoromethoxy)phenylmethanesulfonyl]-ethyl}-3-fluoro-4-methoxybenzamide;
3-bromo-thiophene-2-carboxylic acid-{(R)-1-(cyanomethylcarbamoyl)-2-[2-(1,1-difluoromethoxy)-phenylmethanesulfonyl]ethyl}amide;
3-chlorobenzo[b]thiophene-2-carboxylic acid-{(R)-1-(cyanomethylcarbamoyl)-2-[2-(1,1-difluoromethoxy)phenylmethanesulfonyl]ethyl}amide;
3-chlorothiophene-2-carboxylic acid-{(R)-1-(cyanomethyl-carbamoyl)-2-[2-(1,1-difluoromethoxy)phenylmethanesulfonyl]ethyl}amide;
N-{(R)-(cyanomethylcarbamoyl)-[2-(1,1-difluoromethoxy)phenylmethanesulfonyl]ethyl}-trifluoromethyl-benzamide;
(R)—N-cyanomethyl-3-[2-(1,1-difluoromethoxy)-phenylmethanesulfonyl]-2-(naphthalene-2-sulfonylamino)-propionamide;
cyclopentanecarboxylic acid-{(R)-1-(cyanomethylcarbamoyl)-2-[2-(1,1-difluoromethoxy)-phenylmethanesulfonyl]ethyl}amide;
N-[1R-cyanomethylcarbamoyl-2-(3-trifluoromethoxybenzylsulfonyl)ethyl]benzamide;
N-[1R-cyanomethylcarbamoyl-2-(2-difluoromethoxybenzylsulfonyl)ethyl]benzamide;
N-[1R-cyanomethylcarbamoyl-2-(2-trifluoromethoxybenzylsulfonyl)ethyl]benzamide;
N-(1R-cyanomethylcarbamoyl-2-(3-difluoromethoxybenzylsulfonyl)ethyl]benzamide;
N-[1R-cyanomethylcarbamoyl-2-(2-difluoromethoxybenzylsulfonyl)ethyl]morpholine-4-carboxamide;
N-[1R-(1-cyanocyclopropylcarbamoyl)-2-(2-difluoromethoxybenzylsulfonyl)ethyl]-morpholine-4-carboxamide;
N—[(S)-1-(1-benzooxazol-2-ylmethanoyl)butyl]-3-benzylsulfonyl-2-benzylsulfonylmethylpropionamide;
N—[(S)-1-(1-benzooxazol-2-ylmethanoyl)-butyl]-3-(2-trifluoromethylbenzylsulfonyl)-2-(2-trifluoromethylbenzylsulfonylmethyl)propionamide;
N—[(S)-1-(1-benzooxazol-2-ylmethanoyl)pentyl]-4-(2-methoxybenzenesulfonyl)-2-[2-(2-n methoxybenzenesulfonyl)ethyl]butyramide;
4-benzenesulfonyl-2-(2-benzenesulfonylethyl)-N—[(S)-1-(1-benzooxazol-2-ylmethanoyl)-butyl]butyramide;
(R)—N—[(S)-1-(1-benzooxazol-2-ylmethanoyl)butyl]-2-cyclohexylmethyl-3-benzylsulfonylpropionamide;
N—[(S)-1-(1-benzothiazol-2-ylmethanoyl)propyl]-4-morpholin-4-yl-4-oxo-2-benzylsulfonylmethylbutyramide;
N—[(S)-1-(1-benzooxazol-2-ylmethanoyl)butyl]-3-cyclohexyl-2-cyclohexylmethyl-propionamide;
N—[(S)-1-(1-benzooxazol-2-ylmethanoyl)butyl]-3-isobutylsulfanyl-2-isobutylsulfanylmethylpropionamide;
N—[(S)-1-(1-benzooxazol-2-yl-methanoyl)-butyl]-3-benzylsulfanyl-2-benzylsulfanylmethylpropionamide;
N—[(S)-1-(1-benzooxazol-2-ylmethanoyl)-butyl]-4-phenylsulfanyl-2-(2-phenylsulfanylethyl)butyramide;
N-cyanomethyl-4-morpholin-4-yl-4-oxo-2-(2-trifluoromethylbenzylsulfonylmethyl)-butyramide;
N⁴-(4-carbamoyl-phenyl)-N¹-cyanomethyl-2-benzylsulfonylmethylsuccinamide;
N-cyanomethyl-2-[2-(1,1-difluoromethoxy)benzylsulfonylmethyl]-4-morpholin-4-yl-4-oxobutyramide;
N—[(S)-1-(1-benzooxazol-2-ylmethanoyl)propyl]-4-morpholin-4-yl-4-oxo-2-benzylsulfonylmethylbutyramide;
N—[(S)-1-(1-benzooxazol-2-ylmethanoyl)pentyl]-4-morpholin-4-yl-4-oxo-2-25, benzylsulfonylmethylbutyramide;
(S)-2,2-difluoro-4-(4-morpholin-4-yl-4-oxo-2-benzylsulfonylmethylbutanoylamino)-3-oxo-hexanoic acid dimethylamide;
N—[(S)-1-(1-benzylcarbamoylmethanoyl)propyl]-4-morpholin-4-yl-4-oxo-2-benzylsulfonylmethylbutyramide;
3-biphenyl-3-yl-N-cyanomethyl-2-benzylsulfonylmethylpropionamide;
3-biphenyl-4-yl-N-cyanomethyl-2-benzylsulfonylmethylpropionamide;
3-(3-bromo-phenyl)-N-cyanomethyl-2-benzylsulfonylmethylpropionamide;
N—[(S)-1-((E)-2-benzenesulfonylvinyl)pentyl]-3-benzylsulfonyl-2-benzylsulfonylmethylpropionamide;
N-(3-benzenesulfonyl-1-phenethylallyl)-3-benzylsulfonyl-2-benzylsulfonylmethylpropionamide;
N-cyanomethyl-3-(3-cyanobenzylsulfonyl)-2-benzylsulfonylmethylpropionamide;
4-morpholin-4-yl-4-oxo-2-benzylsulfonylmethyl-N-{(S)-1-[1-(3-phenyl-[1,2,4]oxadiazol-5-yl)-methanoyl]-propyl}-butyramide;
N-cyanomethyl-2-[2-1,1-difluoromethoxy)benzylsulfanylmethyl]-3-benzylsulfanyl-propionamide;
N-cyanomethyl-3-(2-trifluoromethylbenzylsulfanyl)-2-(2-trifluoromethyl-benzylsulfanylmethyl)-propionamide;
N-cyanomethyl-3-isobutylsulfanyl-2-isobutylsulfanylmethylpropionamide;
N-cyanomethyl-4-phenylsulfanyl-2-(2-phenylsulfanylethyl)butyramide;
N-cyanomethyl-3-[2-(1,1-difluoromethoxy)benzylsulfanyl]-2-[2-(1,1-difluoromethoxy)-benzylsulfanylmethyl]propionamide;
3-benzylsulfanyl-2-benzylsulfanylmethyl-N-cyanomethylpropionamide;
N-cyanomethyl-2-[2-1,1-difluoromethoxy)benzylsulfonylmethyl]-3-benzylsulfonylpropionamide;
N-cyanomethyl-3-(2-trifluoromethylbenzylsulfonyl)-2-(2-trifluoromethyl-benzylsulfonylmethyl)propionamide;
4-benzenesulfonyl-2-(2-benzenesulfonylethyl)-N-cyanomethylbutyramide;
N-cyanomethyl-3-[2-(1,1-difluoromethoxy)benzylsulfonyl]-2-[2-(1,1-difluoromethoxy)-benzylsulfonylmethyl]propionamide;
N-cyanomethyl-3-benzylsulfonyl-2-benzylsulfonylmethylpropionamide;
N—[(S)-1-(1-benzylcarbamoylmethanoyl)propyl]-3-benzylsulfonyl-2-benzylsulfonylmethylpropionamide;
N—[(S)-1-(1-benzooxazol-2-ylmethanoyl)butyl]-2-[2-(1,1-difluoromethoxy)-benzylsulfonylmethyl]-3-benzylsulfonylpropionamide;
N-cyanomethyl-3-(2-methylpropane-1-sulfonyl)-2-(2-methylpropane-1-sulfonylmethyl)-propionamide;
acetic acid (2S,3S)-3-(4-morpholin-4-yl-4-oxo-2-benzylsulfonylmethylbutanoylamino)-4-oxo-azetidin-2-yl ester;
N-cyanomethyl-3-(2-methylthiazol-4-ylmethylsulfonyl)-2-benzylsulfonylmethylpropionamide;
N-(3-benzenesulfonylamino-2-oxopropyl)-4-morpholin-4-yl-4-oxo-2-benzylsulfonylmethylbutyramide;
3-biphenyl-3-yl-N-cyanomethyl-2-[2-(1,1-difluoromethoxy)benzylsulfonylmethyl]-propionamide;
(3′-{2-(cyanomethylcarbamoyl)-3-[2-(1,1-difluoromethoxy)benzylsulfonyl]propyl}-biphenyl-4-yl)-carbamic acid ethyl ester;
N-cyanomethyl-2-[2-(1,1-difluoromethoxy)-benzylsulfonylmethyl]-3-(4′-methylsulfonyl-aminobiphenyl-3-yl)propionamide;
3-(3-bromophenyl)-N-cyanomethyl-2-[2-(1,1-difluoromethoxy)phenyl-methylsulfonylmethyl]-propionamide;
N-cyanomethyl-2-((E)-3-phenyl-allyl)-3-benzylsulfonylpropionamide;
N-cyanomethyl-3-benzylsulfonyl-2-(3-phenylpropyl)propionamide;
4-morpholin-4-yl-4-oxo-N-[1-(2-oxo-2-phenylacetyl)pentyl]-2-benzylsulfonylmethylbutyramide;
4-morpholin-4-yl-4-oxo-N-[1-(oxophenylacetyl)pentyl]-2-benzylsulfonylmethylbutyramide as a mixture of diastereomers;
3-(4-morpholin-4-yl-4-oxo-2-benzylsulfonylmethylbutyrylamino)-4-oxo-pyrrolidine-1-carboxylic acid tert-butyl ester;
4-(4-morpholin-4-yl-4-oxo-2-benzylsulfonylmethylbutyrylamino)-3-oxo-azepane-1-carboxylic acid benzyl ester;
N-(1,1-dimethyl-2-oxazolo[4,5-b]pyridin-2-yl-2-oxoethyl)-4-morpholin-4-yl-4-oxo-2-benzylsulfonylmethylbutyramide;
N-[1-(5-ethyl-[1,3,4]oxadiazole-2-carbonyl)butyl]-4-morpholin-4-yl-4-oxo-2-benzylsulfonylmethylbutyramide;
N-[1-(5-ethyl-[1,3,4]oxadiazole-2-carbonyl)butyl]-4-oxo-2-benzylsulfonylmethyl-4-piperidin-1-yl-butyramide;
N-[1-(5-ethyl-[1,3,4]oxadiazole-2-carbonyl)butyl]-4-oxo-2-benzylsulfonylmethyl-4-pyrrolidin-1-yl-butyramide;
N-[1-(5-methoxymethyl-[1,3,4]oxadiazole-2-carbonyl)propyl]-4-morpholin-4-yl-4-oxo-2-benzylsulfonylmethylbutyramide;
N-[1-(5-methoxymethyl-[1,3,4]oxadiazole-2-carbonyl)propyl]-4-oxo-2-benzylsulfonylmethyl-4-piperidin-1-yl-butyramide;
N-[1-(5-methoxymethyl-[1,3,4]oxadiazole-2-carbonyl)-propyl]-4-oxo-2-benzylsulfonylmethyl-4-pyrrolidin-1-yl-butyramide;
4-morpholin-4-yl-4-oxo-2-benzylsulfonylmethyl-N-[1-(5-phenyl-[1,3,4]oxadiazole-2-carbonyl)propyl]butyramide;
4-oxo-2-benzylsulfonylmethyl-N-[1-(5-phenyl-[1,3,4]oxadiazole-2-carbonyl)propyl-4-piperidin-1-ylbutyramide;
4-oxo-2-benzylsulfonylmethyl-N-[1-(5-phenyl-[1,3,4]oxadiazole-2-carbonyl)propyl]-4-pyrrolidin-1-yl-butyramide;
4-morpholin-4-yl-N-[1-(oxazolo[4,5-b]pyridine-2-carbonyl)propyl]-4-oxo-2-benzylsulfonylmethylbutyramide;
N-[1-(oxazolo[4,5-b]pyridine-2-carbonyl)propyl]-4-oxo-2-benzylsulfonylmethyl-4-piperidin-1-yl-butyramide;
N-[1-(oxazolo[4,5-b]pyridine-2-carbonyl)propyl]-4-oxo-2-benzylsulfonylmethyl-4-pyrrolidin-1-ylbutyramide;
4-morpholin-4-yl-4-oxo-2-benzylsulfonylmethyl-N-[1-(5-pyridin-4-yl-[1,3,4]oxadiazole-2-carbonyl)propyl]butyramide;
4-oxo-2-benzylsulfonylmethyl-4-piperidin-1-yl-N-[1-(5-pyridin⁻⁴-yl-[1,3,4]oxadiazole-2-carbonyl)propyl]butyramide;
4-oxo-2-benzylsulfonylmethyl-N-[1-(5-pyridin-4-yl-[1,3,4]oxadiazole-2-carbonyl)-propyl]-4-pyrrolidin-1-ylbutyramide;
4-morpholin-4-yl-4-oxo-2-benzylsulfonylmethyl-N-[1-(5-pyridin-3-yl-[1,3,4]oxadiazole-2-carbonyl)propyl]butyramide;
N-[1-(benzooxazole-2-carbonyl)propyl-4-oxo-2-benzylsulfonylmethyl-4-piperidin-1-yl-butyramide;
N-[1-(benzooxazole-2-carbonyl)propyl]-4-oxo-2-benzylsulfonylmethyl-4-pyrrolidin-1-yl-butyramide;
N-[1-(benzooxazole-2-carbonyl)propyl]-2-cyclohexylmethyl-4-morpholin-4-yl-4-oxo-butyramide;
2-cyclohexylmethyl-4-morpholin-4-yl-N-[1-(oxazolo[4,5-b]pyridine-2-carbonyl)propyl]-4-oxobutyramide;
2-cyclohexylmethyl-N-[1-(5-ethyl-[1,3,4]oxadiazole-2-carbonyl)butyl]-4-morpholin-4-yl-4-oxo-butyramide;
N-(2-benzooxazol-2-yl-1-methoxymethyl-2-oxo-ethyl)-2-(2-difluoromethoxy-benzylsulfonylmethyl)-4-morpholin-4-yl-4-oxo-butyramide;
N-[1-(benzooxazole-2-carbonyl)propyl]-2-(2-cyclohexylethyl)-4-morpholin-4-yl-4-oxo-butyramide;
2-(2-cyclohexylethyl)-4-morpholin-4-yl-N-[1-(oxazolo[4,5-b]pyridine-2-carbonyl)-propyl]-4-oxo-butyramide;
12-(2-cyclohexylethyl)-4-morpholin-4-yl-4-oxo-N-[1-(5-phenyl-[1,3,4]oxadiazole-2-carbonyl)propyl]butyramide;
2-(2-difluoromethoxybenzylsulfonylmethyl)-4-morpholin-4-yl-4-oxo-N-[1-(5-phenyl-[1,3,4]oxadiazole-2-carbonyl)propyl]butyramide;
2-(2-difluoromethoxybenzylsulfonylmethyl)-N-[1-(5-ethyl-[1,3,4]oxadiazole-2-carbonyl)-butyl]-4-morpholin-4-yl-4-oxo-butyramide;
N-[1-(benzooxazole-2-carbonyl)propyl]-2-(2-difluoromethoxybenzylsulfonylmethyl)-4-morpholin-4-yl-4-oxo-butyramide;
2-(2-morpholin-4-yl-2-oxo-ethyl)-5-phenylpentanoic acid, 1-(benzooxazole-2-carbonyl)-propyl]amide;
(R)-2-cyclohexylmethyl-4-morpholin-4-yl-4-oxo-N—[(S)-1-(5-phenyl-1,2,4-oxadiazole-3-carbonyl)propyl]butyramide;
2-(2-morpholin-4-yl-2-oxo-ethyl)-5-phenylpentanoic acid, (S)-1-(5-phenyl-[1,2,4]oxadiazole-3-arbonyl)propyl]amide;
4-morpholin-4-yl-4-oxo-2-benzylsulfonylmethyl-N—[(S)-1-(5-phenyl-1,2,4-oxadiazole-3-carbonyl)propyl]butyramide;
(R)-2-cyclohexylmethyl-4-morpholin-4-yl-4-oxo-N—[(S)-1-(3-phenyl-1,2,4-oxadiazole-5-carbonyl)propyl]butyramide;
4-morpholin-4-yl-N-[1-(oxazole-2-carbonyl)-3-phenylpropyl]-4-oxo-2-benzylsulfonylmethylbutyramide;
N-(1,1-dimethyl-2-oxazol-2-yl-2-oxo-ethyl)-4-morpholin-4-yl-4-oxo-2-benzylsulfonylmethylbutyramide;
N-4-isopropyl-N-1-[1-(oxazole-2-carbonyl)-3-phenylpropyl]-2-benzylsulfonylmethylsuccinamide;
2-(2-difluoromethoxybenzylsulfonylmethyl)-4-morpholin-4-yl-N-[1-(oxazole-2-carbonyl)-3-phenylpropyl]-4-oxo-butyramide;
2-(2-methylpropane-1-sulfonylmethyl)-4-morpholin-4-yl-N-[1-(oxazole-2-carbonyl)-3-phenylpropyl]-4-oxo-butyramide;
2-cyclopropylmethylsulfonylmethyl-4-morpholin-4-yl-N-[1-(oxazole-2-carbonyl)-3-phenylpropyl]-4-oxo-butyramide;
N-[1-(benzooxazole-2-carbonyl)butyl]-2-benzylsulfonyl-3-(tetrahydropyran-4-yl-oxymethyl)-propionamide;
N-[1-(benzooxazole-2-carbonyl)butyl]-3-ethanesulfonyl-2-(tetrahydropyran-4-yl-oxymethyl)-propionamide;
N-(1-benzenesulfonyl-3-oxo-azepan-4-yl)-2-cyclopropylmethylsulfonylmethyl-4-4 morpholin-4-yl-4-oxo-butyramide;
2-cyclopropylmethylsulfonylmethyl-N-{(S)-1-[(R)-hydroxy-(3-phenyl-1,2,4-oxadiazol-5-yl)methyl]propyl}-4-morpholin-4-yl-4-oxo-butyramide;
N-{(S)-1-[(R)-hydroxy-(3-phenyl-1,2,4-oxadiazol-5-yl)methyl]propyl}-2-(2-methylpropane-1-sulfonylmethyl)-4-morpholin-4-yl-4-oxo-butyramide;
2-(2-morpholin-4-yl-2-oxo-ethyl)-5-phenylpentanoic acid
{(S)-1-[(R)-hydroxy-(3-phenyl-1,2,4-oxadiazol-5-yl)methyl]propyl}amide;
2-cyclopropylmethylsulfonylmethyl-4-morpholin-4-yl-4-oxo-N—[(S)-1-(3-phenyl-1,2,4-oxadiazole-5-carbonyl)propyl]butyramide;
2-(2-methylpropane-1-sulfonylmethyl)-4-morpholin-4-yl-4-oxo-N—[(S)-1-(3-phenyl-1,2,4-oxadiazole-5-carbonyl)propyl]butyramide;
2-(2-morpholin-4-yl-2-oxo-ethyl)-5-phenylpentanoic acid,
(S)-1-(3-phenyl-1,2,4-oxadiazole-5-carbonyl)propyl}amide;
3-hydroxy-4-(4-morpholin-4-yl-4-oxo-2-benzylsulfonylmethyl-butyrylamino)-azepane-1-carboxylic acid tert-butyl ester;
4-(2-cyclopropylmethylsulfonylmethyl-4-morpholin-4-yl-4-oxo-butyrylamino)-3-hydroxy-azepane-1-carboxylic acid tert-butyl ester;
3-hydroxy-4-[2-(2-methylpropane-1-sulfonylmethyl)-4-morpholin-4-yl-4-oxo-butyrylamino)-azepane-1-carboxylic acid tert-butyl ester;
4-(4-morpholin-4-yl-4-oxo-2-benzylsulfonylmethyl-butyrylamino)-3-oxo-azepane-1-carboxylic acid tert-butyl ester;
4-(2-cyclopropylmethylsulfonylmethyl-4-morpholin-4-yl-4-oxo-butyrylamino)-3-oxo-azepane-1-carboxylic acid tert-butyl ester;
4-[2-(2-methylpropane-1-sulfonylmethyl)-4-morpholin-4-yl-4-oxo-butyrylamino]-3-oxo-azepane-1-carboxylic acid tert-butyl ester;
N-(1-benzenesulfonyl-3-oxo-azepan-4-yl)-4-morpholin-4-yl-4-oxo-2-benzylsulfonylmethylbutyramide;
N-(1-benzenesulfonyl-3-oxo-azepan-4-yl)-2-(2-methylpropane-1-sulfonylmethyl)-4-morpholin-4-yl-4-oxo-butyramide;
N—[(1S)-1-(benzooxazol-2-ylhydroxymethyl)-3-phenylpropyl]-2-cyclopropylmethylsulfonylmethyl-4-morpholin-4-yl-4-oxo-butyramide;
(R)-2-((S)-1-hydroxy-2-morpholin-4-yl-2-oxo-ethyl)-5-phenylpentanoic acid, 1-(benzoxazole-2-carbonyl)propyl]-amide;
(R)-5-(2-difluoromethoxyphenyl)-2-((S)-1-hydroxy-2-morpholin-4-yl-2-oxo-ethyl)-<pentanoic acid, 1-(benzoxazole-2-carbonyl)-propyl]-amide;
4-morpholin-4-yl-N-[1-(oxazole-2-carbonyl)cyclopropyl]-4-oxo-2-benzylsulfonylmethylbutyramide;
(R)—N-cyanomethyl-2-hydroxy-3-phenylmethanesulfonylpropionamide;
(R)—N-(1-cyano-1-thiophen-2-yl-methyl)-2-hydroxy-3-phenylmethanesulfonylpropionamide;
(R)—N-(1-cyano-1-thiophen-2-yl-methyl)-3-[2-(1,1-difluoromethoxy)phenylmethanesulfonyl]-2-hydroxypropionamide;
(R)—N-cyanomethyl-3-[2-(1,1-difluoromethoxy)phenylmethanesulfonyl]-2-hydroxypropionamide;
morpholine-4-carboxylic acid (R)-1-(cyanomethylcarbamoyl)-2-phenylmethanesulfonylethyl ester;
morpholine-4-carboxylic acid (R)-1-(cyanomethylcarbamoyl)-2-[2-(1,1-difluoromethoxy)phenylmethanesulfonyl]ethyl ester;
(R)-(2-methoxyethyl)-carbamic acid 1-(cyanomethylcarbamoyl)-2-phenylmethanesulfonylethyl ester;
(S)-diethylcarbamic acid 1-(cyanomethylcarbamoyl)-2-cyclohexylethyl ester;
(S)-pyrrolidine-1-carboxylic acid 1-(cyanomethylcarbamoyl)-2-cyclohexylethyl ester;
(S)-morpholine-4-carboxylic acid 1-(cyanomethylcarbamoyl)-2-cyclohexylethyl ester;
(S)-4-ethyl-piperazine-1-carboxylic acid 1-(cyanomethylcarbamoyl)-2-cyclohexylethyl ester;
(S)-2-hydroxymethylpyrrolidine-1-carboxylic acid (S)-1-(cyanomethylcarbamoyl)-2-cyclohexyl-ethyl ester;
(S)-(2,2,2-trifluoroethyl)carbamic acid 1-(cyanomethylcarbamoyl)-2-cyclohexylethyl ester;
(S)-(2-hydroxyethyl)carbamic acid 1-(cyanomethylcarbamoyl)-2-cyclohexylethyl ester;
(tetrahydrofuran-2-ylmethyl)carbamic acid (S)-1-(cyanomethylcarbamoyl)-2-cyclohexylethyl ester;
(S)-azetidine-1-carboxylic acid 1-(cyanomethylcarbamoyl)-2-cyclohexylethyl ester;
(S)-cyclopropylcarbamic acid 1-(cyanomethylcarbamoyl)-2-cyclohexylethyl ester;
(S)-piperidine-1-carboxylic acid 1-(cyanomethylcarbamoyl)-2-cyclohexylethyl ester;
(S)-(2-methoxyethyl)-carbamic acid 1-(cyanomethylcarbamoyl)-2-cyclohexylethyl ester;
(R)-3-hydroxypyrrolidine-1-carboxylic acid (S)-1-(cyanomethylcarbamoyl)-2-cyclohexylethyl ester;
(S)-3-hydroxypyrrolidine-1-carboxylic acid (S)-1-(cyanomethylcarbamoyl)-2-cyclohexylethyl ester;
(S)-morpholine-4-carboxylic acid 1-(cyanomethylcarbamoyl)-3-cyclohexylpropyl ester;
morpholine-4-carboxylic acid (R)-1-[(S)-1-(1-benzooxazol-2-ylmethanoyl)-propylcarbamoyl]-2-phenylmethanesulfonyl-ethyl ester;
morpholine-4-carboxylic acid (R)-1-[(S)-1-(1-benzooxazol-2-ylmethanoyl)propylcarbamoyl]-2-[2-(1,1-difluoromethoxy)phenylmethanesulfonyl]ethyl ester;
morpholine-4-carboxylic acid (R)-1-[(S)-1-(1-benzothiazol-2-ylmethanoyl)propylcarbamoyl]-2-[2-(1,1-difluoromethoxy)phenylmethanesulfonyl]ethyl ester;
pyrrolidine-1-carboxylic acid (R)-1-[(S)-1-(1-benzooxazol-2-ylmethanoyl)propylcarbamoyl]-2-phenylmethanesulfonyl-ethyl ester;
dimethyl-carbamic acid (R)-1-[(S)-1-(1-benzooxazol-2-ylmethanoyl)propylcarbamoyl]-2-phenylmethanesulfonylethyl ester;
morpholine-4-carboxylic acid (R)-1-[(S)-1-(1-benzylcarbamoylmethanoyl)propylcarbamoyl]-2-phenylmethanesulfonylethyl ester;
morpholine-4-carboxylic acid (S)-1-[(S)-1-(oxazolo[4,5-b]pyridine-2-carbonyl)-propylcarbarnoyl]-2-phenylmethanesulfonylethyl ester;
morpholine-4-carboxylic acid (S)-1-[(S)-1-(5-ethyl-[1,3,4]oxadiazole-2-carbonyl)-propylcarbamoyl]-2-phenylmethanesulfonylethyl ester;
(S)-2-{(R)-3-[2-(1,1-difluoromethoxy)phenylmethanesulfonyl]-2-hydroxy-propanoylamino}-N-methoxy-N-methylbutyramide;
(R)-3-[2-(1,1-difluoromethoxy)phenylmethanesulfonyl]-N-((S)-1-formylpropyl)-2-hydroxy-propionamide;
(R)—N—[(S)-1-(1-benzooxazol-2-ylmethanoyl)propyl]-2-hydroxy-3-phenylmethanesulfonylpropionamide;
(S)-3-{3-[2-(1,1-difluoromethoxy)phenylmethanesulfonyl]propanoylamino}-2-oxo-pentanoic acid benzylamide;
N—[(S)-1-(1-benzooxazol-2-ylmethanoyl)-propyl]-3-[2-(1,1-difluoromethoxy)phenylmethanesulfonyl]-propionamide;
N—[(S)-1-(1-benzooxazol-2-ylmethanoyl)-3-phenylpropyl]-3-p-tolylmethanesulfonyl-propionamide;
3-(2-difluoromethoxyphenylmethanesulfonyl)-N-(1-ethyl-2,3-dioxo-3-pyrrolidin-1-yl-propyl)propionamide;
3-(2-difluoromethoxyphenylmethanesulfonyl)-N-(1-ethyl-3-morpholin-4-yl-2,3-dioxopropyl)propionamide;
3-(2-difluoromethoxyphenylmethanesulfonyl)-N-(1-ethyl-2,3-dioxo-3-piperazin-1-yl-propyl)propionamide;
3-(2-difluoromethoxyphenylmethanesulfonyl)-N-[3-(1,1-dioxo-116-thiomorpholin-4-yl)-1-ethyl-2,3-dioxopropyl]propionamide;
3-(2-difluoromethoxyphenylmethanesulfonyl)-N-[1-ethyl-3-(4-methylsulfonylpiperazin-1-yl)-2,3-dioxopropyl]propionamide;
3-[3-(2-difluoromethoxyphenylmethanesulfonyl)propionylamino]-2-oxo-pentanoic acid dimethylamide;
3-[3-(2-difluoromethoxyphenylmethanesulfonyl)propionylamino]-2-oxo-pentanoic acid cyclopentyl-ethyl-amide;
3-[3-(2-difluoromethoxyphenylmethanesulfonyl)propionylamino]-2-oxo-pentanoic acid phenylamide;
3-[3-(2-difluoromethoxyphenylmethanesulfonyl)propionylamino]-2-oxo-pentanoic acid pyridin-3-ylamide;
3-[3-(2-difluoromethoxyphenylmethanesulfonyl)propionylamino]-2-oxo-pentanoic acid (tetrahydro-pyran-4-yl)amide;
3-[3-(2-difluoromethoxyphenylmethanesulfonyl)propionylamino]-2-oxo-pentanoic acid (1-benzoyl-piperidin-4-yl)amide;
3-[3-(2-difluoromethoxyphenylmethanesulfonyl)-propionylamino]-2-oxo-pentanoic acid (2-morpholin-4-ylethyl)amide;
(R)—N—[(S)-1-(1-benzooxazol-2-ylmethanoyl)propyl]-2-(2-nitrophenylamino)-3-phenylmethanesulfonylpropionamide;
N-[1-(benzooxazole-2-carbonyl)propyl]-3-phenylmethanesulfonyl-2-(pyrimidin-2-yl-amino)-propionamide.
(R)—N—[(S)-1-(1-benzooxazol-2-yl-methanoyl)-butyl]-2-(5-nitro-thiazol-2-ylamino)-3-phenylmethanesulfonyl-propionamide;
(2S) (4,4-difluoro-2-hydroxy-5-phenyl-pentanoic acid (1(S)-cyano-3-phenyl-propyl)-amide;
N-(1 (S)-cyano-3-phenyl-propyl)-2-(S)-(2-morpholin-4-yl-2-oxo-ethoxy)-4-phenylbutyramide;
N-(1-(S)-cyano-3-phenylpropyl)-2-(S)-fluoro-4-phenylbutyramide;
N-(1-(S)-cyano-3-phenylpropyl)-2,2-difluoro-4-phenylbutyramide;
N-(1-(S)-cyano-3-phenylpropyl)-2-(S)-hydroxy-4-phenylbutyramide;
N-(1-(S)-cyano-3-phenylpropyl)-2-(R)-hydroxy-4-phenylbutyramide;
N-(1-(S)-cyano-3-phenylpropyl)-2-(R)-methoxy-4-phenylbutyramide;
2,2-difluoro-5-phenylpentanoic acid (1-cyanocyclopropyl)-amide;
N-(1-(S)-cyano-3-phenylpropyl)-4-phenylbutyramide;
2,2-difluoro-5-phenylpentanoic acid ((S)-1-cyano-3-phenylpropyl)amide;
N-(4-cyano-1-ethylpiperidin-4-yl)-3-cyclohexylpropionamide;
N-(4-cyano-1-ethylpiperidin-4-yl)-3-(2-difluoromethoxyphenylmethanesulfonyl)-propionamide;
(S)-tert-butylcarbamic acid 1-(cyanomethylcarbamoyl)-2-cyclohexylethyl ester;
(R)-carbamic acid 1-(cyanomethylcarbamoyl)-2-(2-difluoromethoxyphenylmethanesulfonyl)ethyl ester;
(S)-carbamic acid 1-(cyanomethylcarbamoyl)-2-cyclohexylethyl ester;
(R)-morpholine-4-carboxylic acid 1-(1-cyanocyclopropylcarbamoyl)-2-phenylmethanesulfonylethyl ester;
(R)-morpholine-4-carboxylic acid 1-(4-cyanotetrahydropyran-4-ylcarbamoyl)-2-phenylmethanesulfonylethyl ester;
3-cyclohexyl-2-hydroxy-N-[1-(oxazolo[4,5-b]pyridine-2-carbonyl)propyl]propionamide;
(R)—N-[1-(benzothiazole-2-carbonyl)butyl]-2-isopropylamino-3-phenylmethanesulfonyl-propionamide;
(R)—N-[1-(benzothiazole-2-carbonyl)butyl]-3-phenylmethanesulfonyl-2-(tetrahydropyran-4-ylamino)propionamide;
(R)—N-[1-(benzothiazole-2-carbonyl)butyl]-2-dibenzylamino-3-phenylmethanesulfonyl-propionamide;
(R)—N-[1-(benzothiazole-2-carbonyl)butyl]-2-dimethylamino-3-phenylmethanesulfonyl-propionamide;
(R)—N—[(S)-1-(benzoxazole-2-carbonyl)butyl]-3-phenylmethanesulfonyl-2-(tetrahydropyran-4-ylamino)propionamide;
(R)—N—[(S)-1-(benzoxazole-2-carbonyl)butyl]-2-(1-methylpiperidin-4-ylamino)-3-phenylmethanesulfonylpropionamide;
(R)—N—[(S)-1-(benzoxazole-2-carbonyl)butyl]-2-(bis-thiophen-2-ylmethylamino)-3-phenylmethanesulfonylpropionamide;
(R)—N—[(S)-1-(benzoxazole-2-carbonyl)butyl]-2-dibenzylamino-3-phenylmethanesulfonyl-propionamide;
(S)—N—[(S)-1-(benzoxazole-2-carbonyl)butyl]-2-(tetrahydropyran-4-ylamino)-3-thiophen-2-ylpropionamide;
(S)—N—[(S)-1-(benzoxazole-2-carbonyl)butyl]-2-isopropylamino-3-thiophen-2-yl-propionamide;
(R)—N-[1-(benzothiazole-2-carbonyl)butyl]-3-phenylmethanesulfonyl-2-(tetrahydropyran-4-ylamino)propionamide;
(R)—N—[(S)-1-(benzoxazole-2-carbonyl)butyl]-3-phenylmethanesulfonyl-2-(tetrahydropyran-4-ylamino)propionamide;
(R)—N—[(S)-1-(benzoxazole-2-carbonyl)butyl]-2-isopropylamino-3-phenylmethanesulfonyl-propionamide;
(R)—N—[(S)-1-(benzoxazole-2-carbonyl)butyl]-2-[(2-methoxyethyl)-(tetrahydropyran-4-yl)-amino]-3-phenylmethanesulfonylpropionamide;
(R)—N—[(S)-1-(benzoxazole-2-carbonyl)-butyl]-2-cyclohexylamino-3-phenylmethanesulfonylpropionamide;
(R)—N—[(S)-1-(benzoxazole-2-carbonyl)butyl]-2-dimethylamino-3-phenylmethanesulfonyl-propionamide;
(1S)—N-[1-(benzooxazole-2-carbonyl)butyl]-2-(S)-fluoro-4-phenylbutyramide;
2,2-difluoro-5-phenylpentanoic acid [(S)-1-(benzoxazole-2-carbonyl)butyl]amide;
morpholine-4-carboxylic acid (S)-1-[(S)-1-(benzooxazole-2-carbonyl)propylcarbamoyl]-2-cyclohexyl-ethyl ester;
morpholine-4-carboxylic acid (S)-2-cyclohexyl-1-[(S)-1-(oxazolo[4,5-b]pyridine-2-carbonyl)propylcarbamoyl]ethyl ester;
morpholine-4-carboxylic acid (S)-2-cyclohexyl-1-[(S)-1-(5-ethyl-[1,3,4]oxadiazole-2-carbonyl)propylcarbamoyl]ethyl ester;
morpholine-4-carboxylic acid (S)-2-cyclohexyl-1-[(S)-1-(5-phenyl-[1,3,4]oxadiazole-2-carbonyl)propylcarbamoyl]ethyl ester;
morpholine-4-carboxylic acid (S)-1-[(S)-1-(benzooxazole-2-carbonyl)propylcarbamoyl]-3-cyclohexylpropyl ester;
4-[4,4-dimethyl-2-(morpholine-4-carbonyloxy)pentanoylamino]-3-oxo-azepane-1-carboxylic acid benzyl ester;
(R)—N—[(S)-1-(benzoxazole-2-carbonyl)butyl]-3-cyclopropylmethanesulfonyl-2-(tetrahydropyran-4-ylamino)propionamide;
(R)—N-[1-(benzoxazole-2-carbonyl)-butyl]-2-cyclohexylamino-3-cyclopropylmethane-sulfonylpropionamide;
(R)—N-[1-(benzoxazole-2-carbonyl)-butyl]-2-cycloheptylamino-3-cyclopropylmethane-sulfonylpropionamide;
(R)-3-phenylmethanesulfonyl-N—[(S)-3-phenyl-1-(thiazole-2-carbonyl)propyl]-2-(tetrahydropyran-4-ylamino)propionamide;
(R)—N—[(S)-1-(benzoxazole-2-carbonyl)-3-phenylpropyl]-3-cyclopropylmethanesulfonyl-2-(tetrahydropyran-4-ylamino)propionamide;
(R)-3-cyclopropylmethanesulfonyl-N-[1-(5-ethyl-1,2,4-oxadiazole-3-carbonyl)propyl]-2-(tetrahydropyran-4-ylamino)propionamide;
(R)-3-phenylmethanesulfonyl-N-[1-(3-phenyl-1,2,4-oxadiazole-5-carbonyl)propyl]-2-(tetrahydropyran-4-ylamino)propionamide;
(R)—N-[1-(3-cyclopropyl-1,2,4-oxadiazole-5-carbonyl)-propyl]-3-phenylmethanesulfonyl-2-(tetrahydropyran-4-ylamino)propionamide;
{(R)-1-[1-(benzothiazol-2-yl-hydroxy-methyl)-butylcarbamoyl]-2-phenylmethanesulfonylethyl}carbamic acid tert-butyl ester;
{(R)-1-[(S)-1-(benzoxazol-2-yl-hydroxy-methyl)-butylcarbamoyl]-2-phenylmethanesulfonylethyl}carbamic acid tert-butyl ester;
{(S)-1-[(S)-1-(benzoxazol-2-yl-hydroxymethyl)butylcarbamoyl]-2-thiophen-2-ylethyl}-carbamic acid tert-butyl ester;
{(R)-1-[1-(benzothiazol-2-yl-hydroxy-methyl)-butylcarbamoyl]-2-phenylmethanesulfonylethyl}carbamic acid tert-butyl ester;
{(R)-1-[(S)-1-(benzoxazol-2-yl-hydroxymethyl)butylcarbamoyl]-2-phenylmethanesulfonylethyl}carbamic acid tert-butyl ester;
{(R)-1-[(S)-1-(benzoxazol-2-ylhydroxymethyl)butylcarbamoyl]-2-cyclopropylmethanesulfonylethyl}carbamic acid tert-butyl ester;
(R)-1-{1-[hydroxy-(3-phenyl-1,2,4-oxadiazol-5-yl)methyl]propylcarbamoyl}-2-phenylmethanesulfonylethyl)-carbamic acid tert-butyl ester;
((R)-2-cyclopropylmethanesulfonyl-1-{(S)-1-[(5-ethyl-1,2,4-oxadiazol-3-yl)hydroxymethyl]propylcarbamoyl}ethyl)carbamic acid tert-butyl ester;
{(R)-1-[1-(benzoxazol-2-yl-hydroxymethyl)butylcarbamoyl]-2-phenylmethanesulfonylethyl}carbamic acid tert-butyl ester;
{(R)-1-[(S)-1-(benzoxazol-2-yl-hydroxymethyl)-3-phenylpropylcarbamoyl]-2-cyclopropylmethanesulfonylethyl}-carbamic acid tert-butyl ester;
{(R)-1-[(S)-1-(hydroxythiazol-2-ylmethyl)-3-phenylpropylcarbamoyl]-2-phenylmethanesulfonyl-ethyl}-carbamic acid tert-butyl ester;
{(R)-1-[(S)-1-(benzoxazol-2-yl-hydroxymethyl)butylcarbamoyl]-2-cyclopropylmethanesulfonylethyl}-carbamic acid tert-butyl ester;
(R)-1-{1-[hydroxy-(3-phenyl-1,2,4-oxadiazol-5-yl)methyl]propylcarbamoyl}-2-phenylmethanesulfonylethyl)carbamic acid tert-butyl ester;
((R)-2-cyclopropylmethanesulfonyl-1-{(S)-1-[(5-ethyl-1,2,4-oxadiazol-3-yl)-hydroxymethyl]propylcarbamoyl}ethyl)carbamic acid tert-butyl ester;
{(R)-1-[1-(benzoxazol-2-yl-hydroxymethyl)butylcarbamoyl]-2-phenylmethanesulfonylethyl}carbamic acid tert-butyl ester;
{(R)-1-[(S)-1-(benzoxazol-2-yl-hydroxymethyl)-3-phenylpropylcarbamoyl]-2-cyclopropylmethanesulfonylethyl}-carbamic acid tert-butyl ester;
{(R)-1-[(S)-1-(hydroxythiazol-2-yl-methyl)-3-phenylpropylcarbamoyl]-2-phenylmethanesulfonylethyl}carbamic acid tert-butyl ester;
(R)-2-phenylmethanesulfonyl-1-{(S)-1-[(3-cyclopropyl-1,2,4-oxadiazol-5-yl)hydroxymethyl]propylcarbamoyl}ethyl)carbamic acid tert-butyl ester;
(R)—N-[1-(benzoxazole-2-carbonyl)butyl]-2-[cyclopropylmethyl(tetrahydropyran-4-ylmethyl)amino]-3-phenylmethanesulfonylpropionamide;
(R)—N-[1-(benzothiazol-2-yl-hydroxymethyl)butyl]-2-dibenzylamino-3-phenylmethanesulfonylpropionamide;
(R)—N-[1-(benzothiazol-2-ylhydroxymethyl)butyl]-3-phenylmethanesulfonyl-2-(tetrahydropyran-4-ylamino)propionamide;
(R)—N-[1-(benzothiazol-2-ylhydroxymethyl)butyl]-2-isopropylamino-3-phenylmethanesulfonyl-propionamide;
(R)—N-[1-(benzothiazol-2-ylhydroxymethyl)butyl]-2-dimethylamino-3-phenylmethanesulfonyl-propionamide;
(R)—N—[(S)-1-(benzoxazol-2-ylhydroxymethyl)butyl]-3-phenylmethanesulfonyl-2-(tetrahydropyran-4-ylamino)propionamide;
(R)—N—[(S)-1-(benzoxazol-2-yl-hydroxymethyl)butyl]-2-(1-methylpiperidin-4-ylamino)-3-phenylmethanesulfonylpropionamide;
(R)—N—[(S)-1-(benzoxazol-2-ylhydroxymethyl)butyl]-2-bis-thiophen-2-ylmethylamino)-3-phenylmethanesulfonylpropionamide;
(R)—N—[(S)-1-(benzoxazol-2-ylhydroxymethyl)butyl]-2-dibenzylamino-3-phenylmethanesulfonylpropionamide;
(S)—N—[(S)-1-(benzoxazol-2-ylhydroxymethyl)butyl]-2-(tetrahydropyran-4-ylamino)-3-thiophen-2-yl-propionamide;
a (S)—N—[(S)-1-(benzoxazol-2-ylhydroxymethyl)butyl]-2-isopropylamino-3-thiophen-2-yl-propionamide;
(R)—N—[(S)-1-(benzoxazol-2-yl-hydroxymethyl)butyl]-2-isopropylamino-3-phenylmethanesulfonylpropionamide;
(R)—N-[1-(benzothiazol-2-ylhydroxymethyl)butyl]-3-phenylmethanesulfonyl-2-(tetrahydropyran-4-ylamino)propionamide;
(R)—N—[(S)-1-(benzoxazol-2-ylhydroxymethyl)butyl]-3-phenylmethanesulfonyl-2-(tetrahydropyran-4-ylamino)propionamide;
(R)—N—[(S)-1-(benzoxazol-2-ylhydroxymethyl)butyl]-3-phenylmethanesulfonyl-2-(tetrahydropyran-4-ylamino)-propionamide;
(R)—N—[(S)-1-(benzoxazol-2-ylhydroxymethyl)butyl]-2-[(2-methoxyethyl)-(tetrahydropyran-4-yl)amino]-3-phenylmethanesulfonylpropionamide;
(R)—N—[(S)-1-(benzoxazol-2-ylhydroxymethyl)butyl]-2-cyclohexylamino-3-phenylmethanesulfonyl-propionamide;
(R)—N—[(S)-1-(benzoxazol-2-ylhydroxymethyl)butyl]-2-dimethylamino-3-phenylmethanesulfonyl-propionamide;
N-cyanomethyl-3-cyclohexylpropionamide;
N-cyanomethyl-3-(2-difluoromethoxyphenylmethanesulfonyl)propionamide;
3-(3-cyclohexylpropionylamino)-2-oxo-5-phenylpentanoic acid thiazol-2-ylamide;
3-cyclohexyl-N-(1-formyl-3-phenylpropyl)propionamide;
3-(2-difluoromethoxyphenylmethanesulfonyl)-N—[(S)-1-(5-ethyl-[1,3,4]oxadiazole-2-carbonyl)propyl]propionamide;
N—[(S)-1-(benzooxazole-2-carbonyl)propyl]-2-(2-cyanophenylamino)-3-cyclohexylpropionamide;
N-Cyanomethyl-3-cyclohexyl-2-(4-methoxyphenoxy)propionamide;
2-benzyloxy-N-cyanomethyl-3-cyclohexylpropionamide;
(R)—N—[(S)-1-(1-benzooxazol-2-ylmethanoyl)butyl]-2-benzyloxy-3-phenylmethanesulfonylpropionamide;
(R)—N—[(S)-1-(1-benzooxazol-2-yl-methanoyl)-propyl]-2-methoxymethoxy-3-phenylmethanesulfonylpropionamide;
(S)—N—[(S)-1-(1-benzooxazol-2-ylmethanoyl)butyl]-2-hydroxy-3-phenylpropionamide;
(R)—N—[(S)-1-(1-benzooxazol-2-ylmethanoyl)propyl]-3-phenylmethanesulfonyl-2-triisopropylsilanyloxypropionamide;
(R)—N—[(S)-1-(1-benzothiazol-2-ylmethanoyl)propyl]-2-hydroxy-3-phenylmethanesulfonyl-proplonamide;
(R)-2-hydroxy-3-phenylmethanesulfonyl-N—[(S)-1-(1-pyridazin-3-ylmethanoyl)butyl]-propionamide;
(S)-3-((R)-2-hydroxy-3-phenylmethanesulfonylpropanoylamino)-2-oxo-pentanoic acid benzylamide;
(R)—N—[(S)-1-(1-benzooxazol-2-yl-methanoyl)propyl]-3-[2-(1,1-difluoromethoxy)-phenylmethanesulfonyl]-2-hydroxypropionamide;
(R)—N—[(S)-1-(1-benzothiazol-2-ylmethanoyl)propyl]-3-[2-(1,1-difluoromethoxy)-phenylmethanesulfonyl]-2-hydroxypropionamide;
(2R,5S)-2-[2-(1,1-difluoromethoxy)phenylmethanesulfonylmethyl]-6-ethoxy-5-ethyl-morpholin-3-one;
(S)—N—[(S)-1-(benzoxazol-2-ylcarbonyt)-propyl]-3-cyclohexyl-2-(1,1-dioxobenzo[d]isothiazol-3-ylamino)propionamide;
2-(1,1-dioxobenzo[d]isothiazol-3-ylamino)-4-methyl-pentanoic acid [1-(benzoxazol-2-ylcarbonyl)propyl]amide;
(S)-4-methyl-2-(3-oxo-3H-isoindol-1-ylamino)-pentanoic acid (S)-[1-(benzoxazol-2-yl-carbonyl)propyl]amide;
({1(S)-(1(S)-benzoxazol-2-ylcarbonyl)propylcarbamoyl]-3-methylbutylamino}-morpholin-4-ylmethylene)carbamic acid ethyl ester;
N-[1-(benzoxazol-2-yl-hydroxy-methyl)-propyl]-3-cyclohexyl-2-{[N-(2,2,2-trifluoroethyl)-morpholine-4-carboximidoyl]-amino}-propionamide;
N-[1(S)-(benzoxazol-2-ylcarbonyl)-propyl]-3-phenylmethanesulfonyl-2(R)-{[N-(2,2,2-trifluoroethyl)-morpholin-4-ylcarboximidoyl]amino}propionamide;
N-[1-(benzoxazol-2-ylcarbonyl)propyl]-3-cyclohexyl-2(S)-[N-(2,2,2-trifluoroethyl)-formimidoylamino]propionamide;
N-[1-(benzoxazol-2-ylcarbonyl)propyl]-3-cyclohexyl-2(S)-[(methanesulfonyliminophenyl-methyl)amino]propionamide;
N-[1-(benzoxazol-2-ylcarbonyl)propyl]-2(R)-(1-cyclopentylamino-2-methanesulfonyl-vinylamino)-3-cyclopropylmetanesulfonylpropionamide;
N-[1-(benzoxazol-2-ylcarbonyl)-propyl]-3-cyclohexyl-2(S)-[(morpholin-4-yl-carboximidoyl)amino]propionamide;
N-[1 (S)-(benzoxazol-2-ylhydroxymethyl)propyl]-3-cyclohexyl-2(S)-[(methanesulfonyl-iminomethyl)amino]propionamide;
({1-[1(S)-(benzoxazol-2-ylcarbonyl)-propylcarbamoyl]-2(R)-phenylmethane->sulfonylethylamino}phenylmethylene)carbamic acid ethyl ester;
N—[(R)-1-(cyanomethylcarbamoyl)-2-p-tolylmethanesulfonylethyl]benzamide;
N-(2-benzylsulfonyl-1R-cyanomethylcarbamoylethyl)benzamide;
N-[1R-cyanomethylcarbamoyl-2-(4-methoxybenzylsulfonyl)ethyl]benzamide;
N-(3-phenylsulfonyl-1R-cyanomethylcarbamoylpropyl)benzamide;
morpholine-4-carboxylic acid [(S)-1-(cyanomethylcarbamnoyl)-3-(2-trifluoromethoxybenzenesulfonyl)propyl]amide;
morpholine-4-carboxylic acid [(S)-3-benzenesulfonyl-1-(cyanomethylcarbamoyl)propyl]-amide;
morpholine-4-carboxylic acid [(S)-1-(cyanomethylcarbamoyl)-3-(4-trifluoromethoxybenzenesulfonyl)propyl]amide;
thiophene-2-carboxylic acid [(S)-1-(cyanomethylcarbamoyl)-3-(2-trifluoromethoxybenzenesulfonyl)propyl]amide;
[(S)-1-(cyanomethyl-carbamoyl)-3-(2-trifluoromethoxyphenylsulfanyl)propyl]carbamic acid tert-butyl ester;
3-acetyl-N-(2-benzylsulfonyl-1R-cyanomethylcarbamoylethyl)benzamide;
N-(2-benzylsulfonyl-1R-cyanomethylcarbamoylethyl)naphthalene-2-carboxamide;
N-(2-benzylsulfonyl-1R-cyanomethylcarbamoylethyl)furan-3-carboxamide;
N-(2-benzylsulfonyl-1R-cyanomethylcarbamoylethyl)benzo[1,3]dioxole-5-carboxamide;
N-(2-benzylsulfonyl-1R-cyanomethylcarbamoylethyl)-3-pyridin-3-ylacrylamide;
N-(2-benzylsulfonyl-1R-cyanomethylcarbamoylethyl)benzofuran-2-carboxamide;
N-(2-benzylsulfonyl-1R-cyanomethylcarbamoylethyl)furan-2-carboxamide;
tert-butyl 2-benzylsulfonyl-1R-cyanomethylcarbamoylethylcarbamate;
N-(2-benzylsulfonyl-1R-cyanomethylcarbamoylethyl)-3-phenoxybenzamide;
tert-butyl [3-(2-benzylsulfonyl-1R-cyanomethylcarbamoylethylcarbamoyl)benzyl)-carbamate;
N-(2-benzylsiilfonyl-1R-cyanomethylcarbamoylethyl)-4-hydroxybenzamide;
N-(2-benzylsulfonyl-1R-cyanomethylcarbamoylethyl)-3-hydroxybenzamide;
N-(2-benzylsulfonyl-1R-cyanomethylcarbamoylethyl)thiophene-2-carboxamide;
N-(2-benzylsulfonyl-1R-cyanomethylcarbamoylethyl)thiophene-3-carboxamide;
N-(2-benzylsulfonyl-1R-cyanomethylcarbamoylethyl)quinoline-3-carboxamide;
N-(2-benzylsulfonyl-1R-cyanomethylcarbamoylethyl)biphenyl-4-ylcarboxamide;
N-(2-benzylsulfonyl-1R-cyanomethylcarbamoylethyl)quinoline-2-carboxamide;
4-benzoyl-N-(2-benzylsulfonyl-1R-cyanomethylcarbamoylethyl)benzamide;
N-(2-benzylsulfonyl-1R-cyanomethylcarbamoylethyl)nicotinamide;
N-(2-benzylsulfonyl-1R-cyanomethylcarbamoylethyl)isonicotinamide;
N-(2-benzylsulfonyl-1R-cyanomethylcarbamoylethylmorpholine-4-carboxamide
N-(2-benzylsulfonyl-1R-cyanomethylcarbamoylethyl)-3-methoxybenzamide;
ethyl 4-(2-benzylsulfonyl-1R-cyanomethylcarbamoylethylcarbamoyl)piperazine-1-carboxylate;
tert-butyl 4-(2-benzylsulfonyl-1R-cyanomethylcarbamoylethylcarbamoyl)piperazine-1-carboxylate;
N-(2-benzylsulfonyl-1R-cyanomethylcarbamoylethyl)-4-fur-2-ylcarbonylpiperazine-1-carboxamide;
N-[1R-cyanomethylcarbamoyl-2-(2-nitrobenzylsulfanyl)ethyl]morpholine-4-carboxamide;
N-[2-(4-chlorobenzylsulfonyl)-1R-cyanomethylcarbamoylethyl]benzamide;
N-[1R-cyanomethylcarbamoyl-2-(2-methylbenzylsulfonyl)ethyl]benzamide;
N-[1R-cyanomethylcarbamoyl-2-(3,5-dimethylbenzylsulfonyl)ethyl]benzamide;
N-[1R-cyanomethylcarbamoyl-2-(4-trifluoromethylbenzylsulfonyl)ethyl]benzamide;
N-[1R-cyanomethylcarbamoyl-2-(4-trifluoromethoxybenzylsulfonyl)ethyl]benzamide;
N-[1R-cyanomethylcarbamoyl-2-(4-trifluoromethylsulfanylbenzylsulfonyl)ethyl]-benzamide;
N-[1R-cyanomethylcarbamoyl-2-(3-nitrobenzylsulfonyl)ethyl]benzamide;
N-(1R-cyanomethylcarbamoyl-2-pyridin-2-ylmethylsulfonylethyl)benzamide;
N-(1R-cyanomethylcarbamoyl-2-pyridin-4-ylmethylsulfonylethyl)benzamide;
N-[1R-cyanomethylcarbamoyl-2-(3,4-dichlorobenzylsulfonyl)ethyl]benzamide;
N-[1R-cyanomethylcarbamoyl-2-(3-methylbenzylsulfonyl)ethyl]benzamide;
N-[1R-cyanomethylcarbamoyl-2-(4-nitrobenzylsulfonyl)ethyl]benzamide;
N-[1R-cyanomethylcarbamoyl-2-(2-nitrobenzylsulfonyl)ethyl]benzamide;
N-[1R-cyanomethylcarbamoyl-2-(3-trifluoromethylbenzylsulfonyl)ethyl]benzamide;
N-[1R-cyanomethylcarbamoyl-2-(3-trifluoromethoxybenzylsulfonyl)ethyl]benzamide;
N-(1R-cyanomethylcarbamoyl-2-pyridin-3-ylmethylsulfonylethyl)benzamide;
N-[1R-cyanomethylcarbamoyl-2-(2-methylbenzylsulfonyl)ethyl]morpholine-4-carboxamide;
N-(1R-cyanomethylcarbamoyl)-2-pentafluorobenzylsulfonylethyl)benzamide;
N-(1R-cyanomethylcarbamoyl-2-naphth-2-ylbenzylsulfonylethyl)benzamide;
N-[1R-cyanomethylcarbamoyl-2-(2-fluorobenzylsulfonyl)ethyl]benzamide;
N-[2-(2-chlorobenzylsulfonyl)-1R-cyanomethylcarbamoylethyl]benzamide;
N-(1R-cyanomethylcarbamoyl-2-prop-2-en-1-ylsulfonylethylbenzamide;
N-[2-(2-bromobenzylsulfonyl)-1R-cyanomethylcarbamoylethyl]benzamide;
N-[1R-cyanomethylcarbamoyl-2-(2-iodobenzylsulfonyl)ethyl]benzamide;
N-[2-(4-tert-butylbenzylsulfonyl)-1R-cyanomethylcarbarnoylethyl]benzamide;
N-[1R-cyanomethylcarbamoyl-2-(2-trifluoromethylbenzylsulfonyl)ethyl]benzamide;
N-[1R-cyanomethylcarbamoyl)-2-(2-cyanobenzylsulfonyl)ethyl]benzamide;
N-[2-(4-bromobenzylsulfonyl)-1R-cyanomethylcarbamoylethyl]benzamide;
N-[2-(3-chlorobenzylsulfonyl)-1R-cyanomethylcarbamoylethyl]benzamide;
N-[1R-cyanomethylcarbamoyl-2-(3-fluorobenzylsulfonyl)ethyl]benzamide;
N-[2-(3-chloro-2-fluorobenzylsulfonyl)-1R-cyanomethylcarbamoylethyl]benzamide;
N-[1-cyanomethylcarbamoyl-2-(2-fluoro-3-methylbenzylsulfonyl)ethyl]benzamide;
N-[1-cyanomethylcarbamoyl)-2-(2,5-difluorobenzylsulfonyl)ethyl]benzamide;
N-[1R-cyanomethylcarbamoyl)-2-(4-iodobenzylsulfonyl)ethyl]benzamide;
N-[1R-cyanomethylcarbamoyl)-2-(3-iodobenzylsulfonyl)ethyl]benzamide;
N-[1R-cyanomethylcarbamoyl-2-(2-difluoromethoxybenzylsulfonyl)ethyl]benzamide;
N-[1R-cyanomethylcarbamoyl-2-(2,5-dichlorobenzylsulfonyl)ethyl]benzamide;
N-[2-(3-bromobenzylsulfonyl)-1-cyanomethylcarbamoylethyl]benzamide;
N-[1R-cyanomethylcarbamoyl-2-(3-cyanobenzyl)ethyl]benzamide;
N-[1R-cyanomethylcarbamoyl-2-(4-cyanobenzylsulfonyl)ethyl]benzamide;
N-[1R-cyanomethylcarbamoyl-2-(2-fluoro-6-nitrobenzylsulfonyl)ethyl]benzamide;
N-[2-(2-bromo-5-fluorobenzylsulfonyl)-1R-cyanomethylcarbamoylethyl]benzamide;
N-[1R-cyanomethylcarbamoyl-2-(2,3-difluorobenzylsulfonyl)ethyl]benzamide;
N-[2-biphenyl-2-ylmethylsulfonyl)-1R-cyanomethylcarbamoylethyl]benzamide;
N-[1R-cyanomethylcarbamoyl)-2-(2,4-difluorobenzylsulfonyl)ethyl]benzamide;
N-[1R-cyanomethylcarbamoyl-2-(4-fluorobenzylsulfonyl)ethyl]benzamide;
N-[1R-cyanomethylcarbamoyl-2-(3,4-difluorobenzylsulfonyl)ethyl]benzamide;
N-[1R-eyanomethylcarbamoyl-2-(2,3,4-trifluorobenzylsulfonyl)ethyl]benzamide;
N-[1R-cyanomethylcarbamoyl-2-(2,4,6-trifluorobenzylsulfonyl)ethyl]benzamide;
N-[1R-cyanomethylcarbamoyl-2-(2,4,5-trifluorobenzylsulfonyl)ethyl]benzamide;
N-[1R-cyanomethylcarbamoyl-2-(2,3,6-trifluorobenzylsulfonyl)ethyl]benzamide;
N-[2-(2-chloro-5-trifluoromethylbenzylsulfonyl)-1R-cyanomethylcarbamoylethyl]-benzamide;
N-[2-(2,4-bistrifluoromethylbenzylsulfonyl)-1R-cyanomethylcarbamoylethyl]-benzamide;
“N-[1R-cyanomethylcarbamoyl-2-(2-fluoro-6-trifluoromethylbenzylsulfonyl)ethyl]-benzamide;
N-[1R-cyanomethylcarbamoyl-2-(2-fluoro-3-trifluoromethylbenzylsulfonyl)ethyl]-benzamide;
N-[1R-cyanomrethylcarbamroyl-2-(3-trifluoromethylsulfanylbenzylsulfonyl)ethyl]-benzamide;
N-[1R-cyanomethylcarbamoyl-2-(2-fluoro-4-trifluoromethylbenzylsulfonyl)ethyl]-benzamide;
N-[1R-cyanomethylcarbamoyl-2-(2,3,5-trifluorobenzylsulfonyl)ethyl]benzamide;
N-[1R-cyanomethylcarbamoyl-2-(2-trifluoromethylsulfanylbenzylsulfonyl)ethyl]-benzamide;
N-[1R-cyanomethylcarbamoyl-2-(4-fluoro-2-trifluoromethylbenzylsulfonyl)ethyl]-benzamide;
N-[1R-cyanomethylcarbamoyl-2-(2-fluoro-5-trifluoromethylbenzylsulfonyl)ethyl]-benzamide;
N-[1R-cyanomethylcarbamoyl-2-(2-trifluoromethoxybenzylsulfonyl)ethyl]benzamide;
N-{(R)-1-(cyanomethyl-carbamoyl)-2-[4-(1,1-difluoromethoxy)phenylmethanesulfonyl]-ethyl}-benzamide;
N-[2-(3,5-bistrifluoromethylbenzylsulfonyl)-1R-cyanomethylcarbamoylethyl]-benzamide;
N-[1R-cyanomethylcarbamoyl-2-(2-methoxybenzylsulfonyl)ethyl]benzamide;
N-[1R-cyanomethylcarbamoyl-2-(2,6-dichlorobenzylsulfonyl)ethyl]benzamide;
N-(1R-cyanomethylcarbamoyl-3-pyridin-4-ylsulfonylpropyl)benzamide;
N-(1R-cyanomethylcarbamoyl)-3-pyridin-2-ylsulfonylpropyl)benzamide;
N-(1R-cyanomethylcarbamoyl-2-(3-difluoromethoxybenzylsulfonyl)ethyl]benzamide;
N-[1R-cyanomethylcarbamoyl-2-(4-fluoro-3-trifluoromethylbenzylsulfonyl)ethyl]-benzamide;
4-(2R-benzoylamino-2-cyanomethylcarbamoylethylsulfonylmethyl)benzoic acid;
N-[1R-(1-cyanocyclopropylcarbamoyl)-2-benzylsulfonylethyl]morpholine-4-carboxamide;
N-[1R-(1-cyanocyclopropylcarbamoyl)-2-(2-difluoromethoxybenzylsulfonyl)ethyl]-morpholine-4-carboxamide;
N-[1R-cyanomethylcarbamoyl-2-(2-difluoromethoxybenzylsulfonyl)ethyl]morpholine-4-carboxamide;
morpholine-4-carboxylic acid {(R)-1-(4-cyano-1-methyl-piperidin-4-ylcarbamoyl)-2-[2-(1,1-difluoro-methoxy)-phenylmethanesulfonyl]-ethyl}-amide;
N-[1R-cyanomethylcarbamoyl-2-(3,5-dimethylisoxazol-4-ylmethylsulfony)ethyl]-benzamide;
N-[2-(5-chlorothien-2-ylmethylsulfonyl)-1R-cyanomethylcarbamoylethyl]benzamide;
N-[1R-cyanomethylcarbamoyl-2-(2-fluoro-3-methylbenzylsulfonyl)ethyl]benzamide;
N-[1-(cyanomethylcarbamoyl)-2-(1-oxy-pyridin-2-ylmethanesulfonyl)ethyl]benzamide;
N-{(R)-1-(cyanomethyl-carbamoyl)-2-[2-(1,1-difluoromethoxy)phenylmethanesulfonyl]-ethyl}-1-oxy-nicotinamide;
N-{(R)-1-(cyanomethylcarbamoyl)-2-[2-(1,1-difluoromethoxy)phenylmethanesulfonyl]-ethyl}nicotinamide;
N-{(R)-1-(cyanomethylcarbamoyl)-2-[2-(1,1-difluoromethoxy)phenylmethanesulfonyl]-ethyl}-isonicotinamide;
N-{(R)-1-(cyanomethylcarbamoyl)-2-[2-(1,1-difluoromethoxy)phenylmethanesulfonyl]-ethyl}-1-oxy-isonicotinamide;
pyridine-2-carboxylic acid {(R)-1-(cyanomethylcarbamoyl)-2-[2-(1,1-difluoromethoxy)-phenylmethanesulfonyl]ethyl}amide;
pyrazine-2-carboxylic acid {(R)-1-(cyanomethylcarbamoyl)-2-[2-(1,1-difluoromethoxy)-phenylmethanesulfonyl]ethyl}amide;
N-{(R)-1-(cyanomethylcarbamoyl)-2-[2-(1,1-difluoromethoxy)-phenylmethanesulfonyl]-ethyl}-2-hydroxynicotinamide;
N-{(R)-1-(cyanomethylcarbamoyl)-2-[2-(1,1-difluoromethoxy)phenylmethanesulfonyl]-ethyl}-6-hydroxynicotinamide;
2-amino-N-{(R)-1-(cyanomethylcarbamoyl)-2-[2-(1,1-difluoromethoxy)phenylmethanesulfonyl]ethyl}nicotinamide;
6-amino-N-{(R)-1-(cyanomethylcarbamoyl)-2-[2-(1,1-difluoromethoxy)-phenylmethanesulfonyl]ethyl}nicotinamide;
3-hydroxypyridine-2-carboxylic acid {(R)-1-(cyanomethylcarbamoyl)-2-[2-(1,1-difluoro-methoxy)phenylmethanesulfonyl]ethyl}-amide;
morpholine-4-carboxylic acid {(R)-1-(4-cyanotetrahydropyran-4-ylcarbamoyl)-2-[2-(1,1-difluoromethoxy)phenylmethanesulfonyl]ethyl}amide;
(R)—N-cyanomethyl-3-[2-(1,1-difluoromethoxy)phenylmethanesulfonyl]-2-(3-pyridin-3-yl-ureido)propionamide;
(R)—N-cyanomethyl-3-[2-(1,1-difluoromethoxy)phenylmethanesulfonyl]-2-(3-pyridin-4-yl-ureido)propionamide:
(R)—N-cyanomethyl-3-[2-(1,1-difluoromethoxy)-phenylmethanesulfonyl]-2-(3-isopropyl-ureido)propionamide;
(R)—N-cyanomethyl-3-[2-(1,1-difluoromethoxy)phenylmethanesulfonyl]-2-(3,3-dimethylureido)propionamide;
(R)-2-acetylamino-N-cyanomethyl-3-phenylmethanesulfonylpropionamide;
N—[(R)-1-(cyanomethylcarbamoyl)-2-phenylmethanesulfonylethyl]-2-methoxybenzamide;
N—[(S)-1-(cyanomethylcarbamoyl)-3-phenylmethanesulfonylpropyl]benzamide;
morpholine-4-carboxylic acid {(R)-1-[(1,1-dicyanomethyl)carbamoyl]-2-phenylmethanesulfonylethyl}amide;
2-(2-benzenesulfonylethyl)-N-benzyl-N′-cyanomethylmalonamide;
{(R)-1-(cyanomethylcarbamoyl)-2-[2-(1,1-difluoromethoxy)-phenylmethanesulfonyl]-ethyl}-carbamic acid methyl ester;
{(R)-1-(cyanomethycarbarnoyl)-2-[2-(1,1-difluoromethoxy)phenylmethanesulfonyl]-ethyl}-carbamic acid allyl ester;
{(R)-1-(cyanomethylcarbamoyl)-2-[2-(1,1-difluoromethoxy)phenylmethanesulfonyl]-ethyl}carbamic acid isopropyl ester;
{(R)-1-(cyanomethylcarbamoyl)-2-[2-(1,1-difluoromethoxy)phenylmethanesulfonyl]-ethyl}carbamic acid isobutyl ester;
(R)—N-cyanomethyl-2-(1-oxo-1,3-dihydroisoindol-2-yl)-3-phenylmethanesulfonyl-propionamide;
N-{(R)-1-(cyanomethylcarbamoyl)-2-[2-(1,1-difluoromethoxy)phenylmethanesulfonyl]-ethyl}-3,4-difluorobenzamide;
N-{(R)-1-(cyanomethylcarbamoyl)-2-[2-(1,1-difluoromethoxy)phenylmethanesulfonyl]-ethyl}-3,4-dimethoxybenzamide;
N-{(R)-1-(cyanomethylcarbamoyl)-2-[2-(1,1-difluoromethoxy)phenylmethanesulfonyl]-ethyl}-3-methylbenzamide;
thiophene-3-carboxylic acid {(R)-1-(cyanomethylcarbamoyl)-2-[2-(1,1-difluoromethoxy)-phenylmethanesulfonyl]-ethyl}-amide;
N-{(R)-1-(cyanomethylcarbamoyl)-2-[2-(1,1-difluoromethoxy-phenylmethanesulfonyl]-ethyl}-4-fluorobenzamide;
4-methylpentanoic acid {(R)-1-(cyanomethylcarbamoyl)-2-[2-(1,1-difluoromethoxy)-phenylmethanesulfonyl]-ethyl}amide;
thiophene-2-carboxylic acid {(R)-1-(cyanomethylcarbamoyl)-2-[2-(1,1-difluoromethoxy)-phenylmethanesulfonyl]ethyl}amide;
4-bromo-N-{(R)-1-(cyanomethylcarbamoyl)-2-[2-(1,1-difluoromethoxy)-phenylmethanesulfonyl]ethyl}benzamide;
N-{(R)-1-(cyanomethylcarbamoyl)-2-[2-(1,1-difluoromethoxy)phenylmethanesulfonyl]-ethyl}-4-methoxybenzamide;
N-{(R)-1-(cyanomethylcarbamoyl)-2-[2-(1,1-difluoromethoxy)phenylmethanesulfonyl]-ethyl}-4-trifluoromethoxybenzamide;
naphthalene-2-carboxylic acid {(R)-1-(cyanomethylcarbamoyl)-2-[2-(1,1-difluoromethoxy)-phenylmethanesulfonyl]ethyl}amide;
(E)-N-{(R)-1-(cyanomethylcarbamoyl)-2-[2-(1,1-difluoromethoxy)-phenylmethanesulfonyl]ethyl}-3-phenylacrylamide;
5-methylthiophene-2-carboxylic acid {(R)-1-(cyanomethylcarbamoyl)-2-[2-(1,1-difluoromethoxy)phenylmethanesulfonyl]ethyl}amide;
biphenyl-4-carboxylic acid {(R)-1-(cyanomethylcarbamoyl)-2-[2-(1,1-difluoromethoxy)-phenylmethanesulfonyl]-ethyl}-amide;
1H-indole-5-carboxylic acid {(R)-1-(cyanomethylcarbamoyl)-2-[2-(1,1-difluoromethoxy)phenylmethanesulfonyl]ethyl}amide;
benzo[1,3]dioxole-5-carboxylic acid {(R)-1-(cyanomethylcarbamoyl)-2-[2-(1,1-difluoromethoxy)-phenylmethanesulfonyl]ethyl}amide;
benzo[b]thiophene-2-carboxylic acid {(R)-1-(cyanomethylcarbamoyl)-2-[2-(1,1-difluoromethoxy)-phenylmethanesulfonyl]ethyl}amide;
N-{(R)-1-(cyanomethylcarbamoyl)-2-[2-(1,1-difluoromethoxy)-phenylmethanesulfonyl]-ethyl}-3-phenoxybenzamide;
quinoline-3-carboxylic acid {(R)-1-(cyanomethylcarbamoyl)-2-[2-(1,1-difluoromethoxy)-phenylmethanesulfonyl]-ethyl}amide;
N-{(R)-1-(cyanomethylcarbamoyl)-2-[2-(1,1-difluoromethoxy)phenylmethanesulfonyl]-ethyl}-3-(1-phenyl-methanoyl)-benzamide;
4-chloro-N-{(R)-1-(cyanomethylcarbamoyl)-2-[2-(1,1-difluoromethoxy)phenylmethanesulfonyl]ethyl}benzamide;
N-{(R)-1-(cyanomethylcarbamoyl)-2-[2-(1,1-difluoromethoxy)phenylmethanesulfonyl]-ethyl}-3-fluoro-4-methoxy-benzamide;
3-bromohiophene-2-carboxylic acid {(R)-1-(cyanomethylcarbamoyl)-2-[2-(1,1-difluoromethoxy)phenylmethanesulfonyl]ethyl}-amide;
3-chlorobenzo[b]thiophene-2-carboxylic acid {(R)-1-(cyanomethylcarbamoyl)-2-[2-(1,1-difluoromethoxy)phenylmethanesulfonyl]ethyl}amide;
3-chlorothiophene-2-carboxylic acid {(R)-1-(cyanomethylcarbamoyl)-2-[2-(1,1-difluoromethoxy)phenylmethanesulfonyl]ethyl}amide;
N-{(R)-(cyanomethylcarbamoyl)-[2-(1,1-difluoromethoxy)phenylmethanesulfonyl]ethyl}-trifluoromethylbenzamide;
quinoline-2-carboxylic acid {(R)-1-(cyanomethylcarbamoyl)-2-[2-(1,1-difluoromethoxy)-phenylmethanesulfonyl]ethyl}amide;
(R)-2-benzenesulfonylamino-N-cyanomethyl-3-[2-(1,1-difluoromethoxy)-phenylmethanesulfonyl]propionamide;
(R)—N-cyanomethyl-3-[2-(1,1-difluoromethoxy)phenylmethanesulfonyl]-2-(naphthalene-2-sulfonylamino)propionamide;
(R)—N-cyanomethyl-3-[2-(1,1-difluoromethoxy)phenylmethanesulfonyl]-2-(thiophene-2-sulfonylamino)propionamide;
cyclopentanecarboxylic acid {(R)-1-(cyanomethylcarbamoyl)-2-[2-(1,1-difluoromethoxy)phenylmethanesulfonyl]ethyl}amide;
morpholine-4-carboxylic acid {(R)-1-[(1-cyano-1-thiophen-2-ylmethyl)carbamoyl]-2-phenylmethanesulfonylethyl}amide; and
morpholine-4-carboxylic acid {(R)—-[(1-cyano-1-furan-2-ylmethyl)carbamoyl]-2-phenylmethanesulfonylethyl}amide; or
   a pharmaceutically acceptable salts thereof.

Syntheses of the above specific compounds are described in PCT Applications Publication Nos. WO 00/55144, WO 00/07145, WO 00/55125, WO 01/19796, WO 02/051983, WO 02/098850, WO 02/098406, WO 03/024924, and PCT Application No. US03/19990.

Preferably, the Cathepsin S inhibitor is administered prior to the administration of the biological agent.

Preferably, the Cathepsin S inhibitor is administered concomitantly with the biological agent.

Preferably, the Cathepsin S inhibitor is administered after the administration of the biological agent.

### DETAILED DESCRIPTION OF THE INVENTION

Definitions:

Unless otherwise stated, the following terms used in the specification and claims are defined for the purposes of this Application and have the following meanings.

“Alicyclic” means cycloalkyl and heterocycloalkyl rings as defined herein.

“Alkyl” represented by itself means a straight or branched, saturated aliphatic radical containing one to six carbon atoms, unless otherwise indicated (e.g., alkyl includes methyl, ethyl, propyl, isopropyl, butyl, sec-butyl, isobutyl, tert-butyl, and the like). Alkyl represented along with another radical (e.g., as in arylalkyl) means a straight or branched, saturated aliphatic divalent radical having the number of atoms indicated (e.g., aralkyl includes benzyl, phenethyl, 1-phenylethyl 3-phenylpropyl, and the like). It should be understood that any combination term using an “alk” or “alkyl” prefix refers to analogs according to the above definition of “alkyl”. For example, terms such as “alkoxy” “alkythio” refer to alkyl groups linked to a second group via an oxygen or sulfur atom.

“Alkylene”, unless indicated otherwise, means a straight or branched, saturated aliphatic, divalent radical having the number of one to six carbon atoms, e.g., methylene (—CH₂—), ethylene (—CH₂CH₂—), trimethylene (—CH₂CH₂CH₂—), tetramethylene (—CH₂CH₂CH₂CH₂—) 2-methyltetramethylene (—CH₂CH(CH₃)CH₂CH₂—), pentamethylene (—CH₂CH₂CH₂CH₂CH₂—), and the like.

“Alkylcarbamoyloxy” refers to a radical —OCONHR where R is an alkyl group e.g., methylcarbamoyloxy, ethylcarbamoyloxy, and the like.

“Alkylsulfonylamino” refers to a radical —NHSO₂R where R is an alkyl group e.g., methylsulfonylamino, ethylsulfonylamino, and the like.

“Amino” means the radical —NH₂.

“Aminosulfonyl” refers to a radical —SO₂NH₂.

“Alkylaminosulfonyl” or “dialkylaminosulfonyl” refers to a radical —SO₂NHR and —SO₂NRR′ respectively, where R and R′ are independently alkyl group e.g., methylaminosulfonyl, and the like.

“Alkylaminocarbonyl” or “dialkylaminocarbonyl” refers to a radical —CONHR and —CONRR′ respectively, where R and R′ are independently alkyl group e.g., methylaminocarbonyl, and the like.

“Alkylamino” or “dialkylamino” refers to a radical —NHR and —NRR′ respectively, where R and R′ are independently alkyl group e.g., methylamino, dimethylamino, and the like.

“Alkoxy” refers to a radical —OR where R is an alkyl group e.g., methoxy, ethoxy, and the like.

“Alkoxycarbonyl” refers to a radical —C(O)OR where R is an alkyl group e.g., methoxycarbonyl, ethoxycarbonyl, and the like.

“Alkoxycarbonylalkyl” means the radical -(alkylene)-C(O)OR where R is alkyl as defined above e.g., methoxycarbonylalky, 2-, or 3-ethoxycarbonylmethyl, and the like.

“Alkoxycarbonylamino” refers to a radical —NHC(O)OR where R is an alkyl group e.g., methoxycarbonylamino, ethoxycarbonylamino, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxyalkyl” refers to a radical -(alkylene)-O-(alkylene)-OR where R is an alkyl group e.g., as defined above, e.g., 2-methoxyethyloxymethyl, 3-methoxypropyloxyethyl, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, or —COR^{a} where R^{a} is alkyl, and R′ is hydrogen or alkyl, e.g., aminomethyl, methylaminoethyl, dimethylaminoethyl, 1,3-diaminopropyl, acetylaminopropyl, and the like.

“Alkylthio” refers to a radical —SR where R is an alkyl group e.g., methylthio, ethylthio, and the like.

“Alkylsulfinyl” refers to a radical —S(O)R where R is an alkyl group e.g., methylsylfinyl, ethylsulfinyl, and the like.

“Alkylsulfonyl” refers to a radical —SO₂R where R is an alkyl group e.g., methylsulfonyl, ethylsulfonyl, and the like.

“Acyl” means a radical —COR where R is hydrogen, alkyl, haloalkyl, aryl, aralkyl, heteroaryl, heteroaralkyl, or heterocycloalkyl as defined herein, e.g., formyl, acetyl, trifluoroacetyl, benzoyl, piperazin-1-ylcarbonyl, and the like.

“Alkanoyl” means the radical —COR where R is alkyl as defined above e.g., acetyl, propionyl, and the like.

“Alkanoylamino” means the radical —NHCOR where R is alkyl as defined above e.g., acetylamino, propionylamino, and the like.

“Acyloxy” means a radical —OCOR where R is alkyl, haloalkyl, aryl, aralkyl, heteroaryl, heteroaralkyl, or heterocycloalkyl as defined herein, e.g., acetyloxy, trifluoroacetyloxy, benzoyloxy, piperazin-1-ylcarbonyloxy, and the like.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2.

“Aryl” means a monocyclic or fused bicyclic ring assembly containing 6 to 10 ring carbon atoms unless otherwise indicated, wherein each ring is aromatic e.g., phenyl or naphthyl.

“Aralkyl” means a radical -(alkylene)-R where R is aryl as defined above e.g., benzyl, phenethyl, and the like.

“Aryloxy” means a radical —OR where R is aryl as defined above.

“Aryloxyalkyl” means the radical -(alkylene)-OR where R is aryl as defined above e.g., phenoxymethyl, 2-, or 3-phenoxymethyl, and the like

“Aryloxycarbonyl” means the radical —C(O)OR where R is aryl as defined above e.g., phenyloxycarbonyl, and the like.

“Aralkyloxycarbonyl” means the radical —C(O)OR where R is aralkyl as defined above e.g., benzyloxycarbonyl, and the like.

“Arylcarbamoyloxy” means the radical —OC(O)NHR where R is aryl as defined above e.g., phenylcarbamoyloxy, and the like.

“Aroyl” means the radical —COR where R is aryl as defined above e.g., benzoyl.

“Aroylamino” means the radical —NHCOR where R is aryl as defined above e.g., benzoylamino, and the like.

“Arylthio” refers to a radical —SR where R is an aryl group e.g., phenylthio, and the like.

“Arylsulfinyl” refers to a radical —SOR where R is an aryl group e.g., phenylsulfinyl, and the like.

“Arylsulfonyl” refers to a radical —SO₂R where R is an aryl group e.g., phenylsulfonyl, and the like.

“Aryloxycarbonylamino” refers to a radical —NHC(O)OR where R is an aryl group e.g., phenoxycarbonylamino, and the like.

“Arylsulfonylamino” refers to a radical —NHSO₂R where R is an aryl group as defined above, unless otherwise stated e.g., phenylsulfonylamino, and the like.

“Arylaminosulfonyl” means the radical —SO₂NHR where R is aryl as defined above e.g., phenylaminosulfonyl, and the like.

“Aralkylaminosulfonyl” means the radical —SO₂NHR where R is aralkyl as defined above e.g., benzylaminosulfonyl, and the like.

“Arylaminocarbonyl” means the radical —CONHR where R is aryl as defined above e.g., phenylaminosulfonylarbonyl, and the like.

“Aralkylaminocarbonyl” means the radical —CONHR where R is aralkyl as defined above e.g., benzylaminocarbonyl, and the like.

“Biologic” means a therapeutic agent originally derived from living organisms for the treatment or management of a disease. Examples include, but are not limited to, proteins (recombinant and plasma derived), monoclonal or polyclonal, humanized or murine antibodies, toxins, hormones, and the like. Biologics are currently available for the treatment of a variety of diseases such as cancer, rheumatoid arthritis, and haemophilia.

“Carboxamide” means the radical —C(O)NH₂.

“Carbamoyl” or “aminocarbonyl” means the radical —C(O)NRR′ where R and R′ are independently selected from hydrogen, alkyl, aryl, aralkyl, heteroaryl, heteroaralkyl, heterocycloalkyl or heterocycloalkylalkyl provided one of R and R′ is not hydrogen.

“Carboxy” means the radical —C(O)OH.

“Cycloalkyl” means a monovalent saturated or partially unsaturated, monocyclic, fused bicyclic or bridged polycyclic ring assembly containing three to eight ring carbon atoms e.g., cyclopropyl, cyclobutyl, cyclopentyl, cyclohexyl, cyclohexenyl, 2,5-cyclohexadienyl, bicyclo[2.2.2]octyl, adamantan-1-yl, and the like.

“Cycloalkylalkyl” means the radical -(alkylene)-R where R is cycloalkyl as defined above e.g., cyclopropylmethyl, cyclobutylethyl, cyclobutylmethyl, and the like

“Cycloalkylene” means a divalent saturated or partially unsaturated monocyclic ring or bridged polycyclic ring assembly containing three to eight ring carbon atoms. For example, the instance wherein “R¹ and R^{1a} together with the carbon atom to which both R¹ and R^{1a} are attached form cycloalkylene” includes, but is not limited to, the following:

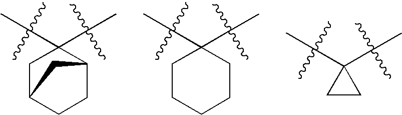

“Disubstituted amino” means a radical —NRR′ where R is alkyl, aryl, aralkyl, heteroaryl, heteraralkyl, or heterocycloalkyl, and R¹ is alkyl, aryl, aralkyl, heteroaryl, heteroaralkyl, cycloalkyl, heterocycloalkyl, cycloalkylalkyl, hydroxyalkyl, alkoxyalkyl, or acyl. Representative examples include, but are not limited to, dimethylamino, methylphenylamino, benzylmethylamino, acetylmethylamino, and the like.

“Disease” specifically includes any unhealthy condition of an animal or part thereof and includes an unhealthy condition that may be caused by, or incident to, medical or veterinary therapy applied to that animal, i.e., the “side effects” of such therapy.

“Deleterious immune response” means an immune response that prevents effective treatment of a patient or causes disease in a patient. As an example, dosing a patient with a murine antibody either as a therapy or a diagnostic agent causes the production of human antimouse antibodies that prevent or interfere with subsequent treatments. The incidence of antibody formation versus pure murine monoclonals can exceed 70%. (see Khazaeli, M. B. et al. J. Immunother. 1994, 15, pp 42-52; Dillman R. O. et al. Cancer Biother. 1994, 9, pp 17-28; and Reinsberg, J. Hybridoma. 1995, 14, pp 205-208). Additional examples of known agents that suffer from deleterious immune responses are blood-clotting factors such as factor VIII. When administered to hemophilia A patients, factor VIII restores the ability of the blood to clot. Although factor VIII is a human protein, it still elicits an immune response in hemophiliacs as endogenous factor VIII is not present in their blood and thus it appears as a foreign antigen to the immune system. Approximately 29-33% of new patients will produce antibodies that bind and neutralize the therapeutically administered factor VIII (see Lusher J. M. Semin Thromb Hemost. 2002, 28(3), pp 273-276). These neutralizing antibodies require the administration of larger amounts of factor VIII in order to maintain normal blood clotting parameters; an expensive regimen of treatment in order to induce immune tolerance (see Briet E et al. Adv. Exp. Med. Bio. 2001, 489, pp 89-97). Another immunogenic example is adenoviral vectors. Retroviral therapy remains experimental and is of limited utility. One reason is that the application of a therapeutic virus generates an immune response capable of blocking any subsequent administration of the same or similar virus (see Yiping Yang et al. J. of Virology. 1995, 69, pp 2004-2015). This ensures that retroviral therapies must be based on the transient expression of a protein or the direct incorporation of viral sequence into the host genome. Directed research has identified multiple viral neutralizing epitopes recognized by host antibodies (see Hanne, Gahery-Segard et al. J. of Virology 1998. 72, pp 2388-2397) suggesting that viral modifications will not be sufficient to overcome this obstacle. This invention will enable a process whereby an adenoviral therapy will have utility for repeated application. Another example of an immunogenic agent that elicits neutralizing antibodies is the well-known cosmetic agent Botox. Botulin toxin protein, is purified from the fermentation of Clostridium botulinum. As a therapeutic agent, it is used for muscle disorders such as cervical dystonia in addition to cosmetic application. After repeated exposure patients generate neutralizing antibodies to the toxin that results in reduced efficacy (see Birklein F. et al. Ann Neurol. 2002, 52, pp 68-73 and Rollnik, J. D. et al. Neurol. Clin. Neurophysiol. 2001, 2001(3), pp 2-4). A “deleterious immune response” also encompasses diseases caused by therapeutic agents. A specific example of this is the immune response to therapy with recombinant human erythropoietin (EPO). Erythropoietin is used to stimulate the growth or red cells and restore red blood cell counts in patients who have undergone chemotherapy or dialysis. A small percentage of patients develop antibodies to EPO and subsequently are unresponsive to both therapeutically administered EPO and their own endogenous EPO (see Casadevall, N. et al., NEJM. 2002, 346, pp 469-475). They contract a disorder, pure red cell aplasia, in which red blood cell production is severely diminished (see Gershon S. K. et. al. NEJM. 2002, 346, pp 1584-1586). This complication of EPO therapy is lethal if untreated. Another specific example is the murine antibody, OKT3 (a.k.a., Orthoclone) a monoclonal antibody directed towards CD-3 domain of activated T-cells. In clinical trials 20-40% of patients administered OKT3 produce antibodies versus the therapy. These antibodies, besides neutralizing the therapy, also stimulate a strong host immune reaction. The immune reaction is severe enough that patients with high titers of human anti-mouse antibodies are specifically restricted from taking the drug (see Orthoclone package label). A final example is a human antibody therapeutic. Humira® is a monoclonal antibody directed against TNF and is used to treat rheumatoid arthritis patients. When taken alone ˜12% of patients develop neutralizing antibodies. In addition, a small percentage of patients given the drug also contract a systemic lupus erthematosus-like condition that is an IgG-mediated immune response induced by the therapeutic agent (see Humira package label).

Another example of “deleterious immune response” is a host reaction to small molecule drugs. It is known to those skilled in the art that certain chemical structures will conjugate with host proteins to stimulate immune recognition (see Ju. C. et al. 2002. Current Drug Metabolism 3, pp 367-377 and Kimber I. et al. 2002, Toxicologic Pathology 30, pp 54-58.) A substantial portion of these host reactions are IgG mediated. Specific “deleterious immune responses” that are IgG mediated include: hemolytic anemia, Steven-Johnson syndrome and drug induced Lupus.

“Halo” means fluoro, chloro, bromo or iodo.

“Haloalkyl” means alkyl substituted by one or more, preferably one to five, “halo” atoms, as such terms are defined in this Application. Haloalkyl includes monohaloalkyl, dihaloalkyl, trihaloalkyl, perhaloalkyl and the like e.g. chloromethyl, dichloromethyl, difluoromethyl, trifluoromethyl, 2,2,2-trifluoroethyl, perfluoroethyl, 2,2,2-trifluoro-1,1-dichloroethyl, and the like).

“Haloalkoxy” refers to a radical —OR where R is haloalkyl group as defined above e.g., trifluoromethoxy, 2,2,2-trifluoroethoxy, difluoromethoxy, and the like.

“Heterocycloalkylene” means cycloalkylene, as defined in this Application, provided that one or more, preferably one or two, of the ring member carbon atoms is replaced by a heteroatom selected from —N—, —O—, —S— or —S(O)₂— and optionally one or two ring member carbon atoms are replaced with —C(O)—. For example, the instance wherein R⁵ and R⁶ together with the carbon atom to which both R⁵ and R⁶ are attached form heterocycloalkylene” includes, but is not limited to, the following:

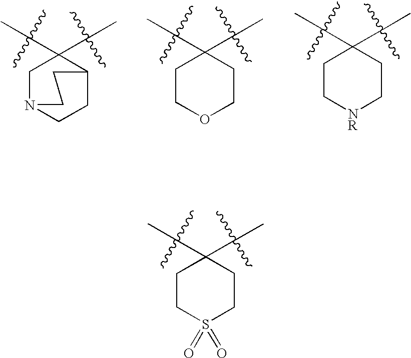

in which R is a substituent defined in the Summary of the Invention

“Heteroaryl” means an aromatic monocyclic or multicyclic ring of 5 to 10 ring atoms in which one or more, preferably one, two, or three, of the ring atoms are selected from nitrogen, oxygen or sulfur, the remaining ring atoms being carbon. Representative heteroaryl rings include, but are not limited to, pyrrolyl, furanyl, thienyl, oxazolyl, isoxazolyl, thiazolyl, imidazolyl, triazolyl, tetrazolyl, pyridinyl, pyrimidinyl, pyrazinyl, pyridazinyl, indolyl, benzofuranyl, benzothienyl, benzimidazolyl, quinolinyl, isoquinolinyl, quinazolinyl, quinoxalinyl, pyrazolyl, and the like.

“Heteroaralkyl” means a radical -(alkylene)-R where R is heteroaryl as defined above e.g:, pyridinylmethyl, 1- or 2-furanylethyl, imidazolylmethyl, and the like.

“Heteroaryloxyalkyl” means the radical -(alkylene)-OR where R is heteroaryl as defined above e.g., furanyloxymethyl, 2-, or 3-indolyloxyethyl, and the like.

“Heteroarylsulfonyl” refers to a radical —SO₂R where R is an heteroaryl group e.g., pyridinylsulfonyl, and the like.

“Heteroarylsulfonylamino” refers to a radical —NHSO₂R where R is an heteroaryl group e.g., pyridinylsulfonylamino, and the like.

“Heteroaralkylsulfonylamino” refers to a radical —NHSO₂R where R is an heteroaralkyl group e.g., pyridinylmethylsulfonylamino, and the like.

“Heteroaryloxycarbonyl” means the radical —C(O)OR where R is heteroaryl as defined above e.g., furanyloxycarbonyl, 2-, or 3-indolyloxycarbonyl, and the like.

“Heteroaralkyloxycarbonyl” means the radical —C(O)OR where R is heteroaralkyl as defined above e.g., furanylmethyloxycarbonyl, 2-, or 3-indolylethykoxycarbonyl, and the like.

“Heterocycloalkyl” means cycloalkyl, as defined in this Application, provided that one or more, preferably one, two, or three of the ring carbon atoms indicated are replaced by a heteroatom selected from —N—, —O—, —S—, —SO—, or —S(O)₂— and additionally one or two carbon atoms are optionally replaced by —C(O). Representative examples include, but are not limited to, imidazolidinyl, morpholinyl, thiomorpholinyl, thiomorpholino-1-oxide, thiomorpholino-1,1-dioxide, tetrahydropyranyl, tetrahydrothiopyranyl, 1-oxo-tetrahydrothiopyranyl, 1,1-dioxotetrathiopyranyl, indolinyl, piperazinyl, piperidyl, pyrrolidinyl, pyrrolinyl, quinuclidinyl, and the like.

“Heterocycloalkylalkyl” means -(alkylene)-heterocycloalkyl as defined in this Application. Representative examples include, but are not limited to, imidazolidin-1-ylmethyl, morpholin-4-ylmethyl, thiomorpholin-4-ylmethyl, thiomorpholin-4-ylmethyl-1-oxide, indolinylethyl, piperazinylmethyl or ethyl, piperidinylmethyl or ethyl, pyrrolidinylmethyl or ethyl, and the like.

“Hydroxy” means the radical —OH.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Isomers” mean compounds of the present invention having identical molecular formulae but differ in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as ether an individual diastereomers or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). It is understood that the names and illustration used in this Application to describe compounds of Formula (Ia) or (Ib) are meant to be encompassed all possible stereoisomers.

Additionally, compounds of Formula (Ia) and (Ib) may exist as tautomers. Such tautomeric forms (individual tautomers or mixtures thereof) are within the scope of this invention. For example, a compound of Formula (Ia) where R² is hydrogen can tautomerize to give a compound of Formula (Ib) where R^{4a} is hydrogen and vice versa as shown below.

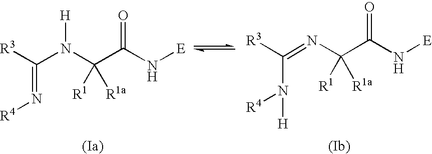

It will be recognized by a person skilled in the art that the amount of tautomers will vary based on certain conditions such as steric interactions, electronic effects of substituents, solvent polarity, hydrogen bonding capababilty, temperature, pH, and the like.

“Cathepsin S inbibitor” is any molecular species which inhibits the transcription of a cathepsin S gene, the processing or translation of a cathepsin S mRNA, or the processing, trafficking or activity of a cathepsin S protein, when administered in vivo or in vitro to a mammalian cell which is otherwise competent to express active cathepsin S. Thus, for example, the term “inhibitor of cathepsin S” embraces a repressor which inhibits induction and/or transcription of the cathepsin S gene, or an antisense sequence which selectively binds to cathepsin S DNA or mRNA sequences and which inhibits the transcription or translation of the cathepsin S sequences. Similarly, the term “inhibitor of cathepsin S” includes competitive, uncompetitive and non-competitive inhibitors of the activity of the cathepsin S protein, such as small molecules which structurally mimic the natural substrates of cathepsin S but which are resistant to the proteolytic activity of the enzyme. Although an inhibitor of cathepsin S may have some degree of inhibitory activity for other genes or proteins which are structurally or functionally related, the term “inhibitor of cathepsin S” is not intended to embrace non-selective suppressors of all gene expression or protein synthesis, or general toxins (e.g., transcription blockers such as actinomycin D, and alpha.-amanitin, protein synthesis inhibitors such as puromycin, cycloheximide, and diptheria toxin).

“Keto or oxo” means the radical (═O).

“Monosubstituted amino” means a radical —NHR where R is alkyl, aryl, aralkyl, heteroaryl, heteloaralkyl, cycloalkyl, cycloalkylalkyl, hydroxyalkyl, alkoxyalkyl, or acyl as defined herein. Representative examples include, but are not limited to, methylamino, phenylamino, benzylamino, cycloalkylmethylamino, acetylamino, trifluoroacetyl, and the like.

“Nitro” means the radical —NO₂.

“Optional” or “optionally” or “may be” means that the subsequently described event or circumstance may or may not occur, and that the description includes instances where the event or circumstance occurs and instances in which it does not. For example, the phrase “wherein the aromatic ring R^{a} is optionally substituted with one or two substituents independently selected from alkyl.” means that the aromatic ring may or may not be substituted with alkyl in order to fall within the scope of the invention.

The present invention also includes N-oxide derivatives of the compounds of this invention. N-oxide derivatives means derivatives of compounds of the present invention in which nitrogens are in an oxidized state (i.e., N→O) e.g., pyridine N-oxide, and which possess the desired pharmacological activity.

“Pathology” of a disease means the essential nature, causes and development of the disease as well as the structural and functional changes that result from the disease processes.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methylsulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

The present invention also includes prodrugs of a compound of the present invention. Prodrug means a compound that is convertible in vivo by metabolic means (e.g. by hydrolysis) to a compound of the present invention. For example an ester of a compound of the present invention containing a hydroxy group may be convertible by hydrolysis in vivo to the parent molecule. Alternatively an ester of a compound of the present invention containing a carboxy group may be convertible by hydrolysis in vivo to the parent molecule. Suitable esters of compounds of the present invention containing a hydroxy group, are for example acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methylsulphonates, ethanesulphonates, benzenesulphonates, p-toluenesulphonates, cyclohexylsulphamates and quinates. Suitable esters of compounds of the present invention containing a carboxy group, are for example those described by Leinweber, F. J. Drug Metab. Res., 1987, 18, pg. 379. An especially useful class of esters of compounds of the present invention containing a hydroxy group, may be formed from acid moieties selected from those described by Bundgaard et al., 3. Med. Chem., 1989, 32, page 2503-2507, and include substituted (aminomethyl)-benzoates, for example, dialkylamino-methylbenzoates in which the two alkyl groups may be joined together and/or interrupted by an oxygen atom or by an optionally substituted nitrogen atom, e.g. an alkylated nitrogen atom, more especially (morpholinomethyl)benzoates, e.g. 3- or 4-(morpholinomethyl)-benzoates, and (4-alkylpiperazin-1-yl)benzoates, e.g. 3- or 4-(4-alkylpiperazin-1-yl)benzoates.

“Protected derivatives” means derivatives of compounds of the present invention in which a reactive site or sites are blocked with protecting groups. Protected derivatives of compounds of the present invention are useful in the preparation of compounds of the present invention or in themselves may be active cathepsin S inhibitors. A comprehensive list of suitable protecting groups can be found in T. W. Greene, Protecting Groups in Organic Synthesis, 3rd edition, John Wiley & Sons, Inc. 1999.

“Tissue graft” means both homograft and xenograft tissue therapies.

“Therapeutically effective amount” means that amount which, when administered to an animal for treating a disease, is sufficient to effect such treatment for the disease.

“Treatment” or “treating” means any administration of a Cathepsin S inhibitor of the present invention and includes:

(1) preventing the immune response from occurring in an animal which may be predisposed to the immune response but does not yet experience or display the pathology or symptomatology of the immune response,

(2) inhibiting the immune response in an animal that is experiencing or displaying the pathology or symptomatology of the immune response (i.e., arresting further development of the pathology and/or symptomatology), or

(3) ameliorating the immune response in an animal that is experiencing or displaying the pathology or symptomatology of the immune response (i.e., reducing in degree or severity, or extent or duration, the overt manifestations of the immune response or reversing the pathology and/or symptomatology e.g., reduced binding and presenation of antigenic peptides by MHC class II molecules, reduced activation of T-cells and B-cells, reduced humoral and cell-mediated responses and, as appropriate to the particular immune response, reduced inflammation, congestion, pain, necrosis, reduced loss in the efficacy of a biologic agent, and the like).

### PREFERRED EMBODIMENTS

While the broadest definition of this invention is set forth in the Summary of the Invention, certain compounds of this invention are preferred. For example:

(I). A preferred group of compounds is of Formula (Ia) and (Ib):

Within this group (I):

A. One preferred group of compounds is that wherein E is —C(R⁵)(R⁶)X¹ in which:

R⁵ is hydrogen or alkyl; and

R⁶ is hydrogen, alkyl, -(alkylene)-OR¹² (where R¹² is hydrogen, alkyl or haloalkyl), cycloalkyl, cycloalkylalkyl, aryl, aralkyl, heteroaryl, heteroaralkyl, heterocycloalkyl, or heterocycloalkylalkyl wherein the aromatic or alicyclic ring in aryl, aralkyl, heteroaryl, heteroaralkyl, heterocycloalkyl or heterocycloalkylalkyl is optionally substituted with one, two, or three R^{a} independently selected from alkyl, haloalkyl, alkoxy, hydroxy, haloalkoxy, halo, carboxy, alkoxycarbonyl, amino, monsubstituted amino, disubstituted amino, or acyl.

Preferably, R⁵ is hydrogen;

R⁶ is alkyl, preferably ethyl; and

X¹ is —CHO, —C(O)R¹⁰, —C(O)CF₃, —C(O)CF₂CF₂R⁹—CH═CHS(O)₂R¹⁰, —C(O)CF₂C(O)NR¹⁰R¹¹, —C(O)C(O)NR¹⁰R¹¹, —C(O)CH₂OR¹⁰, —C(O)CH₂N(R¹¹)SO₂R¹⁰, —C(O)C(O)N(R¹¹)(CH₂)₂OR¹¹, —C(O)C(O)N(R¹¹)(CH₂)₂NHR¹¹ or —C(O)C(O)R¹⁰ wherein R¹⁰ is alkyl, aryl, aralkyl, heteroaryl, heteroaralkyl, cycloalkylalkyl or heterocycloalkylalkyl wherein the aromatic ring is optionally substituted with R^{d} selected from heteroaryl, aryl, or alkyl, R¹¹ is hydrogen or alkyl and R⁹ is halo.

Preferably, E is —CHR⁶C(O)R¹⁰ where R⁶ is alkyl, preferably ethyl, propyl, or butyl, more preferably ethyl, and R¹⁰ is heteroaryl optionally substituted with one or two R^{d} independently selected from alkyl, haloalkyl, alkoxy, alkoxyalkyl, cycloalkyl, hydroxy, haloalkoxy, halo, carboxy, alkoxycarbonyl, aryl, heteroaryl, amino, monsubstituted amino, disubstituted amino, or acyl wherein the aromatic or alicyclic ring in R^{d} is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino, more preferably R¹⁰ is benzoxazol-2-yl, 4-azabenzoxazol-2-yl, 2-pyridin-3-yl-[1,3,4]-oxadiazol-5-yl, 2-pyridin-4-yl-[1,3,4]-oxadiazol-5-yl, 2-ethyl-[1,3,4]-oxadiazol-5-yl, 2-isopropyl-[1,3,4]-oxadiazol-5-yl, 2-tert-butyl-[1,3,4]-oxadiazol-5-yl, 2-phenyl-[1,3,4]-oxadiazol-5-yl, 2-methoxymethyl-[1,3,4]-oxadiazol-5-yl, 2-furan-2-yl-[1,3,4]-oxadiazol-5-yl, 2-thien-2-yl-1,3,4)-oxadiazol-5-yl, 2-(4-methoxyphenyl)-[1,3,4]-oxadiazol-5-yl, 2-(2-methoxyphenyl)-[1,3,4]-oxadiazol-5-yl, 2-(3-methoxyphenyl)-[1,3,4]-oxadiazol-5-yl, 2-(2-trifluoromethoxyphenyl)-[1,3,4]-oxadiazol-5-yl, 2-(3-trifluoromethoxyphenyl)-[1,3,4]-oxadiazol-5-yl, 2-(4-trifluoromethoxyphenyl)-[1,3,4]-oxadiazol-5-yl, 2-(4-dimethylaminophenyl)-[1,3,4]-oxadiazol-5-yl, pyradizin-3-yl, pyrimidin-2-yl, 3-phenyl-[1,2,4]-oxadiazol-5-yl, 3-ethyl-[1,2,4]-oxadiazol-5-yl, 3-cyclopropyl-[1,2,4]-oxadiazol-5-yl, 3-thien-3-yl-[1,2,4]-oxadiazol-5-yl, 3-pyridin-4-yl-[1,2,4]-oxadiazol-5-yl, 3-pyridin-2-yl-[1,2,4]-oxadiazol-5-yl, 5-ethyl-[1,2,4]-oxadiazol-3-yl, 5-phenyl-[1,2,4]-oxadiazol-3-yl, 5-thien-3-yl-[1,2,4]-oxadiazol-3-yl, 5-trifluoromethyl-[1,2,4]-oxadiazol-3-yl, 5-pyridin-4-yl-[1,2,4]-oxadiazol-3-yl, or 5-phenyloxazol-2-yl.

B. Another preferred group of compounds is that wherein E is —C(R⁵)(R⁶)X¹ in which R⁵ and R⁶ taken together with the carbon atom to which both R⁵ and R⁶ are attached form cycloalkylene or heterocycloalkylene, preferably cyclopropylene, cyclopentylene, cyclohexylene, tetrahydropyran-4-yl, tetrahydrothiopyran-4-yl, tetrahydrothiopyran-4-yl-1-oxide, tetrahydrothiopyran-4-yl-1,1-dioxide, or piperidin-4-yl wherein the nitrogen atom is optionally substituted with alkyl or hydroxy, preferably tetrahydrothiopyran-4-yl-1,1-dioxide, and X¹ is —CHO, —C(O)R¹⁰, —C(O)CF₃, —C(O)CF₂CF₂R⁹—CH═CHS(O)₂R¹⁰, —C(O)CF₂C(O)NR¹⁰R¹¹, —C(O)C(O)NR¹⁰R¹¹, —C(O)CH₂OR¹⁰, —C(O)CH₂N(R¹¹)SO₂R¹⁰, —C(O)C(O)N(R¹¹)(CH₂)OR¹¹, —C(O)C(O)N(R¹¹)(CH₂)₂NR¹⁰R¹¹ or —C(O)C(O)R¹⁰. More preferably, —C(O)C(O)NR¹⁰R¹¹ where R¹¹ is hydrogen and R¹⁰ is benzyl.
C. Yet another preferred group of compounds is that wherein E is a group of formula (a): 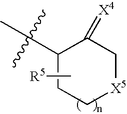
in which:

n is 0, 1, or 2, X⁴ is —NR²²—, —O— or —S— where R²² is hydrogen, alkyl, or alkoxy; X⁵ is —O—, —S(O)₂—, —S— or —NR²³— where R²³ is selected from hydrogen, alkyl, —S(O)₂R²⁴, —C(O)OR²⁶, or acyl, —where R²⁴ is alkyl, haloalkyl, cycloalkyl, cycloalkylalkyl, heterocycloalkyl, heterocycloalkylalkyl, aryl, aralkyl, heteroaryl, or heteroaralkyl and R²⁶ is hydrogen or alkyl. Preferably, X⁴ is —O—, n is 0 or 1, and X⁵ is —O—.

D. Yet another preferred group of compounds is that wherein E is —CR^{5a}R^{6a}CN wherein R^{5a} and R^{6a} are hydrogen.

E. Within this group another preferred group of compounds is that wherein E is —CR^{5a}R^{6a}CN wherein R^{5a} and R^{6a} together with the carbon atom to which they are attached form cycloalkylene optionally substituted with one or two R^{b} independently selected from alkyl, halo, dialkylamino, aryl, aralkyl, cycloalkyl, cycloalkylalkyl, heteroaryl, heteroaralkyl, alkoxycarbonyl, or aryloxycarbonyl. Preferably, R^{5a} and R^{6a} together with the carbon atom to which they are attached form cyclopropylene, cyclobutylene, cyclopentylene, or cyclohexylene optionally substituted with groups described immediately above. More preferably, R^{5a} and R^{6a} together with the carbon atom to which they are attached form cyclopropylene, cyclobutylene, cyclopentylene, cyclohexylene, cycloheptylene, 2-methylcyclopropylene, 3-benzylcyclopentylene, 3-cyclohexylmethylcyclopentylene, 3-cyclopentylmethylcyclopentylene, 3-phenylcyclopentylene, 3-cyclohexylcyclopentylene, 3-cyclopentylcyclopentylene, 3-pyridin-2-ylmethylcyclopentylene, 3-pyridin-3-ylmethylcyclopentylene, 3-pyridin-4-ylmethylcyclopentylene, 2-methylcyclopropylene, 2,3-dimethylcyclopropylene, 3-benzylcyclobutylene, 3-methylcyclopentylene, 3,4-dimethylcyclopentylene, 3-ethylcyclopentylene, 3-(1,1-dimethylpropyl)-cyclopentylene, 3-n-butylcyclopentylene, 3-ethoxycarbonylcyclopentylene, 3,4-diethoxycarbonyl-cyclopentylene, or 3-benzyl-4-dimethylaminocyclopentylene. Most preferably, R^{5a} and R^{6a} together with the carbon atom to which they are attached form cyclopropylene.

F. Yet another preferred group of compounds is that wherein E is —CR^{5a}R^{6a}CN wherein R^{5a} and R^{6a} together with the carbon atom to which they are attached form heterocycloalkylene optionally substituted with one to four alkyl or one or two R^{c} which are independently selected from alkyl, haloalkyl, hydroxyalkyl, alkoxyalkyl, alkoxyalkyloxyalkyl, aryloxyalkyl, heteroaryloxyalkyl, aminoalkyl, acyl, aryl, aralkyl, heteroaryl, heteroaralkyl, heterocycloalkyl, heterocycloalkylalkyl, cycloalkyl, cycloalkylalkyl, —S(O)ₙ₂R¹⁴, -alkylene-S(O)ₙ₂—R¹⁵, —COOR¹⁶, -alkylene-COOR¹⁷, —CONR¹⁸R¹⁹, or -alkylene-CONR²⁰R²¹ (where n2 is 0-2 and R¹⁴—R¹⁷, R¹⁸ and R²⁰ are independently hydrogen, alkyl, haloalkyl, aryl, aralkyl, heteroaryl, heteroaralkyl, cycloalkyl, cycloalkylalkyl, or heterocycloalkyl and R¹⁹ and R²¹ are independently hydrogen or alkyl) wherein the aromatic or alicyclic ring in the groups attached to heterocycloalkylene is optionally substituted with one, two, or three substituents independently selected from alkyl, haloalkyl, cycloalkyl, cycloalkylalkyl, benzyl, alkoxy, hydroxy, haloalkoxy, halo, carboxy, alkoxycarbonyl, amino, monsubstituted amino, disubstituted amino, or acyl. Preferably, R^{5a} and R^{6a} together with the carbon atom to which they are attached form pyrrolidinyl, piperidinyl, tetrahydropyranyl, tetrahydrothiopyranyl, tetrahydrofuranyl, tetrahydrothiopyran-4-yl-1-oxide, tetrahydrothiopyran-4-yl-1,1-dioxide, hexahydropyridmidinyl, or hexahydropyridazinyl optionally substituted as described above. More preferably, R^{5a} and R^{6a} together with the carbon atom to which they are attached form piperidin-4-yl substituted with one to three alkyl or one R^{c} selected from haloalkyl, aminoalkyl, alkoxycarbonyl, alkoxyalkyl, alkoxyalkyloxyalkyl, heterocycloalkyl, heterocycloalkylalkyl, -alkylene-CONR²⁰R²¹, or cycloalkyl wherein the alicyclic ring is optionally substituted with substitutents listed above. Most preferably, R^{5a} and R^{6a} together with the carbon atom to which they are attached form piperidin-4-yl optionally substituted at the 1-position with methyl, ethyl, propyl, n-butyl, n-pentyl, 3-dimethylaminopropyl, 4-dimethylaminobutyl, 3-morpholin-4-ylpropyl, 3-piperidin-1-yl-propyl, 3-(4-methylpiperazin-1-yl)propyl, 3-(1-methylpiperidin-4-yl)propyl, 4-morpholin-4-ylbutyl, 2-(2-methoxyethyloxy)ethyl, 4-methoxybutyl, 4-aminocarbonylbutyl, 3-aminocarbonylpropyl, morpholin-4-yl, 4-methylpiperazin-1-yl, 1-ethoxycarbonylpiperidin-4-yl, 1,1-dioxotetrahydrothiopyran-4-yl, hydroxy, 2,2,2-trifluoroethyl, or tert-butyl, 1,2-dimethylpiperidin-4-yl, 1,2,6-trimethylpiperidin-4-yl, 1,2,2-trimethylpiperidin-4-yl, 1-methyl-2-oxopiperidin-4-yl, 1-methylpiperidin-3-yl, 1-tert-butoxycarbonylpiperidin-4-yl, 1-cyclohexylpiperidin-4-yl, 1-cyclopropylmethylpyrrolidin-3-yl, 1-benzylpyrrolidin-3-yl, 1-benzyloxycarbonylpyrrolidin-3-yl, pyrrolidin-3-yl, 1-hydroxypyrrolidin-3-yl, 1-methylpyrrolidin-3-yl, 1-ethypyrrolidin-3-yl, 1-n-propyl or n-butylpyrrolidin-3-yl, 1-cyclohexylpyrrolidin-3-yl, 1-ethyl-2,2-dimethylpyrrolidin-4-yl, 1-propyl-2-methoxycarbonylpiperidin-4-yl, 2-oxopyrrolidin-3-yl, 1-ethyl-2-oxopyrrolidin-3-yl, morpholin-4-yl, T-(1-methylpiperidin-4-ylcarbonyl)piperidin-4-yl, 1-ethoxycarbonylpiperidin-4-yl, 1-benzylazetidin-3-yl, tetrahydrothiopyran-4-yl-1-oxide, or tetrahydrothiopyran-4-yl-1,1-dioxide.

(a) Within the above preferred and more preferred groups (A-F), a more preferred group of compounds is that wherein:

R^{1a} is alkyl, haloalkyl, cycloalkyl, aryl, aralkyl, heteroaralkyl, cycloalkylalkyl, heterocycloalkylalkyl, or -alkylene-X²—R³² [wherein X² is —NR³³—, —O—, —S(O)ₙ₄—, —CO—, —COO—, —OCO—, —NR³³CO—, —CONR³³, —NR³³SO₂—, —SO₂NR³³—, —NR³³COO—, —OCONR³³—, —NR³³CONR³⁴, or —NR³³SO₂NR³⁴—(where R³³ and R³⁴ are independently hydrogen, alkyl, or acyl and n4 is 0-2) and R³² is hydrogen, alkyl, haloalkyl, cycloalkyl, cycloalkylalkyl, heterocycloalkyl, aryl, aralkyl, heteroaryl, heteroaralkyl, heterocycloalkyl, or heterocycloalkylalkyl] wherein said alkylene chain is optionally substituted with one to six halo and wherein the aromatic or alicyclic ring in R^{1a} is optionally substituted with one, two, or three R^{e} independently selected from alkyl, haloalkyl, alkoxy, hydroxy, haloalkoxy, halo, nitro, cyano, carboxy, alkoxycarbonyl, aryl, heteroaryl, cycloalkyl, cycloalkylalkyl, aralkyl, heteroaralkyl, amino, monsubstituted amino, disubstituted amino, or acyl; and

R¹ and R² are hydrogen.

Preferably, R^{1a} is 2-methylpropyl, 2,2-dimethylpropyl, 3,3-dimethylbutyl, 3-methylbutyl, 2,2,3-trimethylbutyl, 3,3-dimethylpentyl, 3-ethyl-3-methylpentyl, n-butyl, 2-methylbutyl, or 1-methylpropyl.

Preferably, R^{1a} is 4,4-dimethylcyclohexylmethyl, 4-ethyl-4-methylcyclohexylmethyl, 4,4-diethylcyclohexylmethyl, 3,3-dimethylcyclohexylmethyl, 3,5-dimethylcyclohexylmethyl, cycloheptylmethyl, cyclooctylmethyl, 2-cyclohexylethyl, 2-cyclohexyl-2-methylpropyl, 2-(1-methylcyclohexyl)ethyl, 2-(1-methylcyclopropyl)ethyl, 2-(1-methylcyclopropyl)-2-methylpropyl, 2-cyclopentylethyl, 2-cyclopentyl-2-methylpropyl, 4-isopropyl-4-methylcyclohexylmethyl, 2-methylcyclohexylmethyl, 4-methoxycyclohexylmethyl, 1-methylcyclopentylmethyl, cyclohexyl, cyclohexylmethyl, 1,4-dimethylcyclopentylmethyl, cyclohexylethyl, cyclohexylmethyl, cyclopentylmethyl, 1-methylcyclohexylmethyl, 1-methylcyclopentylmethyl, or 1-benzylcyclopropylmethyl, preferably 1-methylcyclopentylmethyl.

Preferably, R^{1a} is 2-bicylo[2.2.1]hep-3-tylethyl, 8-methyl-8-aza-bicyclo[3.2.1]oct-3-ylmethyl, bicyclo[3.2.1]oct-3-ylmethyl, bicyclo[3.1.1)hept-3-ylmethyl, 6,6-dimethylbicyclo[3.1.1]hept-3-ylmethyl, 6,6-dimethylbicyclo[3.1.1]hept-4-ylmethyl, 2-bicyclo[2.2.1]hept-1-ylethyl, or bicyclo[2.2.1]hept-2-ylethyl.

Preferably, R^{1a} is benzyl, 4-methoxybenzyl, 4-dimethylaminobutyl, 2-dimethylaminocarbonylethyl, dimethylaminocarbonylmethyl, methoxycarbonylmethyl, 3,4-dichlorobenzyl, 2-chlorobenzyl, 4-ethoxybenzyl, 4-nitrobenzyl, biphen-4-ylmethyl, naphth-1-ylmethyl, naphth-2-ylmethyl, 4-chlorobenzyl, 3-chlorobenzyl, 4-fluorobenzyl, 2-phenethyl, 4-hydroxybenzyl, 2-(4-hydroxyphenyl)ethyl, 2,6-difluorobenzyl, 2,2-difluoro-3-phenylpropyl, 2,2-dichloro-3-phenylpropyl, 2,2,2-trichloroethyl, 2,2-dichloroethyl, biphenyl-3-ylmethyl, naphth-2-yl, 3-phenylpropyl, 2,2-difluoro-3-phenylpropyl, or 2,2-dimethyl-3-phenylpropyl.

Preferably, R^{1a} is phenylmethanethiomethyl, phenylmethanesulfinylmethyl, ethylthiomethyl, ethylsulfinylmethyl, ethylsulfonylmethyl, isopropylthiomethyl, 2-methylthioethyl, 2-methylsulfinylethyl, 2-methysulfonylethyl, tert-butylthiomethyl, 2-fluorophenylmethane-sulfonylmethyl, 2-chlorophenylmethanesulfonylmethyl, 2-nitrophenylmethanesulfonylmethyl, 2-cyanophenylmethanesulfonylmethyl, pyridin-3-ylmethanesulfonylmethyl, pyridin-2-ylmethanesulfonylmethyl, pyridin-4-ylmethanesulfonylmethyl, 2-fluorophenylmethanethiomethyl, 2-chlorophenylmethanethiomethyl, 2-cyanophenylmethanethiomethyl, 2-nitrophenylmethanethiomethyl, cyclohexylmethanethiomethyl, cyclohexylsulfinylthiomethyl, cyclohexylmethanesulfonylmethyl, thiophene-2-sulfonylmethyl, 3-chloro-2-fluorophenylmethane-sulfonylmethyl, benzenesulfonylmethyl, phenylmethanesulfonylmethyl, 2-benzenesulfonylethyl, 2-(pyridin-2-ylsulfonyl)ethyl, 2-(pyridin-4-ylsulfonyl)ethyl, 2-phenylmethanesulfonylethyl, oxypyridin-2-ylmethanesulfonylmethyl, 4-methoxyphenyl-methanesulfonylmethyl, p-tolylmethanesulfonylmethyl, 4-chlorophenylmethanesulfonylmethyl, o-tolylmethanesulfonylmethyl, 3,5-dimethylphenylmethanesulfonylmethyl, 4-trifluoromethylphenylmethanesulfonylmethyl, 4-trifluoromethoxyphenylmethanesulfonylmethyl, 2-bromophenylmethanesulfonylmethyl, naphth-2-ylmethanesulfonylmethyl, m-tolylmethanesulfonylmethyl, 3-trifluoromethylphenylmethanesulfonylmethyl, 3-trifluoromethoxyphenylmethane-sulfonylmethyl, 4-fluoro-2-trifluoromethoxyphenylmethanesulfonylmethyl, 2-fluoro-6-trifluoromethylphenylmethanesulfonylmethyl, 3-chlorophenylmethanesulfonylmethyl, 2-trifluoromethylphenylmethanesulfonylmethyl, 4-tert-butylphenylmethanesulfonylmethyl, 2-fluoro-3-methylphenylmethanesulfonyl-methyl, 3-fluorophenylmethanesulfonylmethyl, 4-fluorophenylmethanesulfonylmethyl, 2,5-difluorophenylmethanesulfonylmethyl, 2,6-difluorophenylmethanesulfonylmethyl, 2,5-dichlorophenylmethanesulfonylmethyl, 3,4-dichlorophenylmethanesulfonylmethyl, 2-(1,1-difluoromethoxy)phenylmethanesulfonylmethyl, 3-cyanophenylmethanesulfonylmethyl, 2-trifluoromethoxyphenylmethanesulfonylmethyl, 2,3-difluorophenylmethane-sulfonylmethyl, biphenyl-2-ylmethane-sulfonylmethyl, cyclohexylmethyl, 3,4-difluorophenylmethanesulfonylmethyl, 2,4-difluorophenylmethanesulfonylmethyl, 2,4,6-trifluorophenylmethanesulfonylmethyl, 2,4,5-trifluorophenylmethanesulfonylmethyl, 2,3,4-trifluorophenylmethanesulfonylmethyl, 2,3,5-trifluorophenylmethanesulfonylmethyl, 2,5,6-trifluorophenylmethanesulfonyl-methyl, 2-chloro-5-trifluoromethylphenylmethanesulfonylmethyl, 2-methylpropane-1-sulfonylmethyl, 2-fluoro-3-trifluoromethylphenylmethanesulfonylmethyl, 2-fluoro-4-trifluoromethylphenylmethanesulfonylmethyl, 2-fluoro-5-trifluoromethylphenylmethanesulfonylmethyl, 4-fluoro-3-trifluoromethylphenylmethanesulfonylmethyl, 2-methoxyphenylmethanesulfonylmethyl, 3,5-bis-trifluoromethylphenylmethanesulfonylmethyl, 4-difluoromethoxyphenylmethane-sulfonylmethyl, 3-difluoromethoxyphenylmethane-sulfonylmethyl, 2,6-dichlorophenylmethanesulfonylmethyl, biphenyl-4-ylmethanesulfonylmethyl, 3,5-dimethyl-isoxazol-4-ylmethanesulfonylmethyl, 5-chlorothien-2-ylmethane-sulfonylmethyl, 2-[4-(1,1-difluoromethoxy)benzenesulfonyl]ethyl, 2-[2-(1,1-difluoromethoxy)benzenesulfonyl]ethyl, 2-[3-(1,1-difluoromethoxy)benzenesulfonyl]ethyl, 2-(4-trifluoromethoxybenzenesulfonyl)ethyl, 2-(3-trifluoromethoxybenzenesulfonyl)-ethyl, 2-(2-trifluoromethoxybenzenesulfonyl)ethyl, isobutylsulfanylmethyl, 2-phenylsulfanylethyl, 2-cyclohexylethanesulfonylmethyl, phenylmethanesulfanylmethyl, 2-trifluoromethylphenylmethanesulfanylmethyl, phenylsulfanylethyl, cyclopropylmethanesulfonylmethyl, 2-methylpropylsulfonylmethyl, or 3,4,5-trimethoxy-phenylmethanesulfonylmethyl, preferably 2-(1,1-difluoromethoxy)phenylmethanesulfonylmethyl.

Preferably, R^{1a} is 1-ethoxycarbonylpiperidin-4-ylmethyl, 1-methylpiperidin-4-ylmethyl, 2-tetrahydropyran-4-ylethyl, pyrrolidin-1-ylmethyl, piperidin-1-ylmethyl, morpholin-4-ylmethyl, 1-morpholin-4-ylethyl, thiomorpholin-4-ylmethyl, 1-oxo-thiomorpholin-4-ylmethyl, 1,1-dioxothiomorpholin-4-ylmethyl, tetrahydrothiopyran-4-ylmethyl, 1-oxotetrahydrothiopyran-4-ylmethyl, 1,1-dioxotetrahydrothiopyran-4-ylmethyl, 1-methylpiperazin-4-ylmethyl, benzyloxymethyl, ethoxymethyl, isopropyloxymethyl, 2-dimethylaminoethyl, 2-piperidin-1-ylethyl, 2-pyrrolidin-1-ylethyl, tert-butyloxymethyl, imidazol-4-ylmethyl, indol-3-ylmethyl, 2-pyrrolidin-1-ylcarbonylethyl, pyrrolidin-1-ylcarbonylmethyl, indol-2-ylmethyl, 1-benzylimidazol-4-ylmethyl, 4-ethyl-4-methytpiperidin-1-ylmethyl, indol-1-ylmethyl, 1-methylpiperidin-2-ylmethyl, 2,2,-difluoro-3-thien-2-ylmethyl, or pyridin-4-ylmethyl.

Preferably, R^{1a} is cyclohexyl, 2-cyclohexylethyl, cyclohexylmethyl, tert-butylmethyl, 1-methylcyclohexylmethyl, 1-methylcyclopentylmethyl, 2,2-difluoro-3-phenylpropyl, 2,2-dichloro-3-phenyl propyl, 2,2,2-trichloroethyl, 2,2-dichloroethyl, 1,4-dimethylcyclopentyl]methyl, 2,2-dimethyl-3-phenyl propyl, 1-benzyl cyclopropylmethyl, 2-(1,1-difluoromethoxy)phenylmethanesulfonylmethyl, 2-(1,1-difluoromethoxy)phenylmethaneoxy-methyl, pyridin-4-ylmethyl, phenylmethanesulfonylmethyl, pyridin-2-ylmethanesulfonylmethyl, pyridin-4-ylmethanesulfonylmethyl, 2-methylpropylsulfonylmethyl, cyclopropylmethanesulfonylmethyl, pyridin-3-ylmethanesulfonylmethyl, 2,6-difluorophenylmethanesulfonylmethyl, 2-pyridin-2-ylsulfonylethyl, 2-phenylsulfonylethyl, benzyloxymethyl, 2,2-dimethylpropyl, cyclopentylmethyl, morpholin-4-ylmethyl, 5-bromothien-2-ylmethyl, pyridin-4-ylmethyl, 2-chlorobenzyl, or 4-fluorobenzyl; most preferably 1-methylcyclopentylmethyl; and

R¹ and R² are hydrogen.

(b) Yet another more preferred group of compounds within groups (A-F) is that wherein R¹ and R^{1a} together with the carbon atoms to which they are attached form cycloalkylene or heterocycloalkylene, preferably 3,3-dimethylcyclobutyl, cyclohexyl, cyclopentyl, cyclooctyl, tetrahydrothiopyran-1,1-dioxide, or piperidin-4-yl wherein the nitrogen atom at the 1-position of the piperidinyl ring is optionally substituted with R^{f} where R^{f} is alkyl or —SO₂R where is alkyl, cycloalkyl, cycloalkylalkyl, aryl, aralkyl, heteroaryl or heteroaralkyl where the rings in R^{f} are optionally substituted with one, two, or three substitutents independently selected from alkyl, alkoxy, haloalkyl, haloalkoxy, hydroxy, halo, or carboxy.

(1) Within the above preferred, more preferred, and even more preferred groups above, a particularly preferred group of compounds is that wherein:

R³ is hydrogen, alkyl, cycloalkyl, phenyl, benzyl, naphthyl, alkylSO₂alkyl, cycloalkylSO₂alkyl, arylSO₂alkyl, pyrrolidinyl, piperidinyl, morpholinyl, thiomorpholinyl, piperazinyl, indolinyl, pyranyl, thiopyranyl, furanyl, thienyl, pyrrolyl, oxazolyl, thiazolyl, imidazolyl, pyridinyl, isoxazolyl, pyrimidinyl, pyrazinyl, pyridazinyl, indolyl, quinolinyl, benzofuranyl, benzthienyl, benzimidazolyl, benzthiazolyl, benzoisoxazolyl, benzoxazolyl, amino, alkylamino, or dialkylamino; wherein the aromatic or alicyclic ring in R³ is optionally substituted by one, two, or three R^{g};

each R^{g} is independently alkyl, halo, hydroxy, oxo, carboxy, cyano, nitro, carboxamide, cycloalkyl, phenyl, naphthyl, pyrrolidinyl, piperidinyl, morpholinyl, thiomorpholinyl, piperazinyl, furanyl, thienyl, oxazolyl, thiazolyl, imidazolyl, triazolyl, tetrazolyl, pyridinyl, pyrimidinyl, pyrazinyl, indolyl, benzofuranyl, benzothienyl, benzimidazolyl, benzthiazolyl, benzoxazolyl, quinolinyl, isoquinolinyl, quinazolinyl, quinoxalinyl, alkoxy, —COR (where R is alkyl), —OC(O)R (where R is alkoxy or aryl), aryloxy, benzyloxy, alkoxycarbonyl, aryloxycarbonyl, carbamoyl wherein the nitrogen atom may be independently mono or di-substituted by alkyl, aryl, pyrrolidinyl, piperidinyl, morpholinyl, thiomorpholinyl, piperazinyl, furanyl, thienyl, oxazolyl, thiazolyl, imidazolyl, triazolyl, tetrazolyl, pyridinyl, pyrimidinyl, pyrazinyl, indolyl, benzofuranyl, benzothienyl, benzimidazolyl, benzthiazolyl, quinolinyl, isoquinolinyl, quinazolinyl or quinoxalinyl, —NHCOR (where R is alkyl or aryl), alkylthio, arylthio, alkylsulfinyl, alkylsulfonyl, arylsulfinyl, arylsulfonyl, alkoxycarbonylamino, aryloxycarbonylamino, alkylcarbamoyloxy, arylcarbamoyloxy, alkylsulfonylamino, arylsulfonylamino, alkylaminosulfonyl, arylaminosulfonyl, or amino wherein the nitrogen atom may be independently mono or di-substituted by alkyl, aryl, pyrrolidinyl, piperidinyl, morpholinyl, thiomorpholinyl, piperazinyl, furanyl, thienyl, oxazolyl, thiazolyl, imidazolyl, triazolyl, tetrazolyl, pyridinyl, pyrimidinyl, pyrazinyl, indolyl, benzofuranyl, benzothienyl, benzimidazolyl, benzthiazolyl, quinolinyl, isoquinolinyl, quinazolinyl or quinoxalinyl, where the aromatic or alicyclic rings in R^{g} may be further optionally substituted by one, two or three R^{h} independently selected from alkyl, alkoxy, haloalkyl, haloalkoxy, halo, hydroxy, carboxy, carboxamido, cyano, nitro, aryl or cycloalkyl;

R² is hydrogen or methyl;

R⁴ is hydrogen, hydroxy, nitrile, -(alkylene)-X⁶—R³⁸ (where X⁶ is —O—, NR³⁹—, —S(O)ₙ₇—, —NR³⁹CO—, —CO—, or —OC(O)— where R³⁹ is hydrogen or alkyl and R³⁸ is hydrogen, alkyl, phenyl, naphthyl, pyrrolidinyl, piperidinyl, morpholinyl, thiomorpholinyl, piperazinyl, indolinyl, pyranyl, thiopyranyl, furanyl, thienyl, pyrrolyl, oxazolyl, isoxazolyl, thiazolyl, imidazolyl, pyridinyl, pyrimidinyl, pyrazinyl, indolyl, benzofuranyl, benzothienyl, benzimidazolyl, benzthiazolyl, quinolinyl, isoquinolinyl, quinazolinyl, benzoxazolyl, or quinoxalinyl; and

R^{4a} is hydrogen, alkyl, cycloalkyl, aminoalkyl, aryl, alkoxy, aryloxy, benzyloxy, or —C(O)OR where (R is hydrogen, alkyl, alkoxyalkyl, haloalkyl, cycloalkyl, cycloalkylalkyl, heterocycloalkyl, heterocycloalkylalkyl, heteroaryl, heteroaralkyl, aryl, or aralkyl).

Preferably, R³ is hydrogen, methyl, ethyl, isopropyl, cyclopropyl, cyclopentyl, cyclohexyl, phenyl, benzyl, naphthyl, pyrrolidinyl, piperidinyl, morpholinyl, thiomorpholinyl, piperazinyl, furanyl, thienyl, thiazolyl, imidazolyl, pyridinyl, pyrazinyl, or amino where the nitrogen atom is mono or disubstituted with alkyl, and wherein the aromatic or alicylic rings in R³ are optionally substituted with one, two, or three R^{g} independently selected from methyl, ethyl, fluoro, chloro, bromo, iodo, hydroxy, oxo, carboxy, cyano, nitro, carboxamide, cyclopropyl, phenyl, pyrrolidinyl, piperidinyl, morpholinyl, thiomorpholinyl, piperazinyl, thienyl, imidazolyl, methoxy, acetyl, acetoxy, phenoxy, benzyloxy, methoxycarbonyl, phenoxycarbonyl, carbamoyl wherein the nitrogen atom is mono or disubstituted independently with methyl, ethyl or phenyl, acetylamino, benzoylamino, methylthio, phenylthio, phenylsulfonyl, methylsulfonyl, methoxycarbonylamino, phenoxycarbonylamino, methylcarbamoyloxy, phenylcarbamoyloxy, methylsulfonylamino, phenylsulfonylamino, methylaminosulfonyl, phenylaminosulfonyl, or amino wherein the nitrogen atom is mono or disubstituted independently with methyl or phenyl; wherein the aromatic or alicyclic rings in R^{g} are further optionally substituted with one, two, or three R^{h} independently selected from methyl, cyclopropyl, phenyl, methoxy, fluoro, chloro, hydroxy, carboxy, or carboxamido.

Even more preferably, R³ is hydrogen, methyl, ethyl, isopropyl, cyclopropyl, cyclohexyl, phenyl, naphthyl, benzyl, pyrrolidinyl, piperidinyl, morpholinyl, thiomorpholinyl, furanyl, thienyl, thiazolyl, imidazolyl, pyridinyl, pyrazinyl or amino where the nitrogen atom is mono or disubstituted with alkyl and wherein the aromatic or alicyclic rings in R³ are optionally substituted with one, two, or three R^{g} independently selected from methyl, fluoro, chloro, phenyl, thienyl, methoxy, acetyl, acetoxy, phenoxy, benzyloxy, methoxycarbonyl, carbamoyl wherein the nitrogen atom is mono or disubstitued independently with methyl or phenyl, acetylamino, methylthio, phenylthio, phenylsulfonyl, methylsulfonyl, methoxycarbonylamino, methylcarbamoyloxy, phenylcarbamoyloxy, methylsulfonylamino, phenylsulfonylamino, amino wherein the nitrogen atom is mono or disubstituted independently with methyl or phenyl. Most preferably, R³ is hydrogen, isopropyl, cyclohexyl, phenyl, 4-(acetylamino)phenyl, 4-methanesulfonylaminophenyl, 4-methoxyphenyl, 3-phenoxyphenyl, 4-chlorophenyl, 4-fluorophenyl, 2-fluorophenyl, 2-fluoro-4-chlorophenyl, naphthyl, thienylmethyl, piperidinyl, morpholinyl, thiomorpholinyl, furanyl, thienyl, pyridin-4-yl, pyrazinyl, methylamino, ethylamino, dimethylamino or diethylamino.

Within the above preferred and more preferred R³ groups, R⁴ is hydrogen and

R^{4a} is hydrogen, alkyl or alkoxy; preferably, hydrogen; or

R^{4a} is C(O)OR, preferably ethoxycarbonyl, 2-methylpropyloxycarbonyl, 2,2,-dimethylpropyloxy-carbonyl, methoxycarbonyl, cyclopentyloxycarbonyl, propyloxycarbonyl, hexyloxycarbonyl, 3-methoxybutyloxycarbonyl, 2-isobutyloxy-ethyloxycarbonyl, isopropyloxycarbonyl, benzyloxycarbonyl, cyclohexylmethyloxy-carbonyl, pyran-4-ylmethyloxycarbonyl, tetrahydrofuran-3-yloxycarbonyl, 2-methoxyethoxycarbonyl, 3,3,3-trifluoropropyloxycarbonyl, cyclobutylmethyloxycarbonyl, cyclobutoxycarbonyl, piperidin-4-ylmethoxycarbonyl, 3-pyrrolidin-1-ylpropyloxycarbonyl, 3-piperidin-1-ylpropyloxycarbonyl, 3-dimethylpropyl-oxycarbonyl, 2-dimethylaminoethyloxycarbonyl, 2-pyridin-4-ylethyloxycarbonyl, or 2-(4-methylpiperazin-1-ylethyloxycarbonyl.

(2) Within the above preferred, more preferred, and even more preferred groups above, yet another particularly preferred group of compounds is that wherein:

R² and R^{4a} are hydrogen;

R³ is hydrogen, alkyl, haloalkyl, phenyl, pyrrolidinyl, piperidinyl, morpholinyl, thiomorpholinyl, piperazinyl, furanyl, thienyl, pyridinyl, or amino; wherein R³ is optionally substituted by one, two or three R^{g} where each R^{g} is independently halo or alkyl. Preferably, R³ is methyl, trifluoromethyl, morpholin-4-yl, pyrrolidin-1-yl, piperidin-1-yl, 4-methylpiperazin-1-yl, thien-2-yl, thien-3-yl, furan-2-yl, furan-3-yl, pyridin-2-yl, pyridin-3-yl, pyridin-4-yl, pyrimidin-5-yl, pyrimidin-4-yl, oxazol-4-yl, oxazol-5-yl, thiazol-4-yl, thiazol-5-yl, quinolin-6-yl, indol-5-yl, 2-methylimidazol-4-yl, phenyl, or 4-fluorophenyl; and

R⁴ is hydrogen, alkyl, or halogenated alkyl, preferably, hydrogen, 2,2,2-trifluoroethyl or methyl.

(3) Within the above preferred, more preferred, and even more preferred groups above, yet another particularly preferred group of compounds is that wherein:
R³ and R⁴ in (Ia) and (Ib) together with the atoms to which they are attached form a 5, 6, or 7 membered heterocycloalkyl ring, preferably a heterocycloalkyl ring containing at least an —SO₂— group or a 5, 6, or 7 membered heterocycloalkyl ring containing at least an —SO₂— group that is fused to an aryl or heteroaryl ring wherein each ring is optionally independently substituted by one or two R^{j} where each R^{j} is independently halo, alkoxy, haloalkyl, haloalkoxy, hydroxy or alkyl.

More preferably, R³ and R⁴ in (Ia) and (Ib) together with the atoms to which they are attached form a 5 or 6 membered heterocycloalkyl ring containing at least an —SO₂— group or a 5 or 6 membered heterocycloalkyl ring containing at least an —SO₂— group and is fused to a thienyl or pyrrolyl ring optionally independently substituted by one or two R^{j} as defined in the paragraph above.

Even more preferably, R³ and R⁴ in (Ia) and (Ib) together with the atoms to which they are attached form a 5 or 6 membered heterocycloalkyl ring containing at least an —SO₂— group or a 5 or 6 membered heterocycloalkyl ring containing at least an —SO₂— group and is fused to a phenyl or pyridinyl ring optionally independently substituted by one or two R^{j} as defined in the paragraph above.

Most preferably, R³ and R⁴ in (Ia) and (Ib) together with the atoms to which they are attached form a ring of formula: 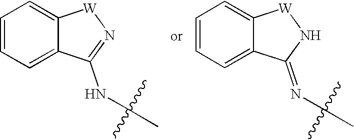
wherein W is —S(O)₂— wherein each ring is optionally independently substituted by one or two R^{j} where each R^{j} is independently chloro, fluoro, methoxy, trifluoromethyl, trifluoromethoxy, hydroxy, or methyl.

Within the above preferred and more preferred groups in (3), a particularly preferred group of compounds is that where R² is hydrogen.

(4) Within the above preferred, more preferred, and even more preferred groups above, yet another particularly preferred group of compounds is that wherein:

R³ and R⁴ in (Ia) and (Ib) together with the atoms to which they are attached form a 5, 6, or 7 membered heterocycloalkyl ring fused to an aryl or heteroaryl ring wherein said heterocyclic ring is substituted on the aromatic and/or non-aromatic portion of the rings with one, two, or three R^{j} provided that the heterocycloalkyl ring does not contain an —SO₂— group;

each R^{j} is independently alkyl, cycloalkyl, aryl, alkoxy, aryloxy, benzyloxy, alkoxycarbonyl where each of the aforementioned groups is optionally substituted with halo, haloalkyl, alkyl, alkoxy, haloalkoxy, hydroxy, oxo, carboxy, nitrile, nitro, or —C(O)NH₂.

Most preferably, R³ and R⁴ in (Ia) and (Ib) together with the atoms to which they are attached form a ring of formula:

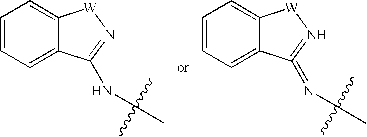

wherein W is —O—C(O)—, —CO—, or —NR—C(O)— (where R is hydrogen, alkyl, alkoxycarbonylalkyl, alkylsulfonylalkyl, alkylaminoalkyl, or dialkylaminoalkyl) wherein each ring is independently substituted by one or two R^{j}

each R^{j} is independently chloro, fluoro, methoxy, trifluoromethyl, trifluoromethoxy, hydroxy, methyl, or phenyl where the phenyl ring is optionally substituted with one, two or three substituents independently selected from chloro, fluoro, methyl, methoxy, trifluoromethyl, trifluoromethoxy, or hydroxy. Preferably R^{j} is phenyl where the phenyl ring is optionally substituted with one, two or three substituents independently selected from chloro, fluoro, methyl, methoxy, trifluoromethyl, trifluoromethoxy, or hydroxy.

Within the above preferred and more preferred groups in (4), a particularly preferred group of compounds is that where R² is hydrogen.

(5) Within the above preferred, more preferred and even more preferred groups, yet another particularly preferred group of compounds is that wherein R³ and R⁴ together with the atoms to which they are attached form a group selected from:

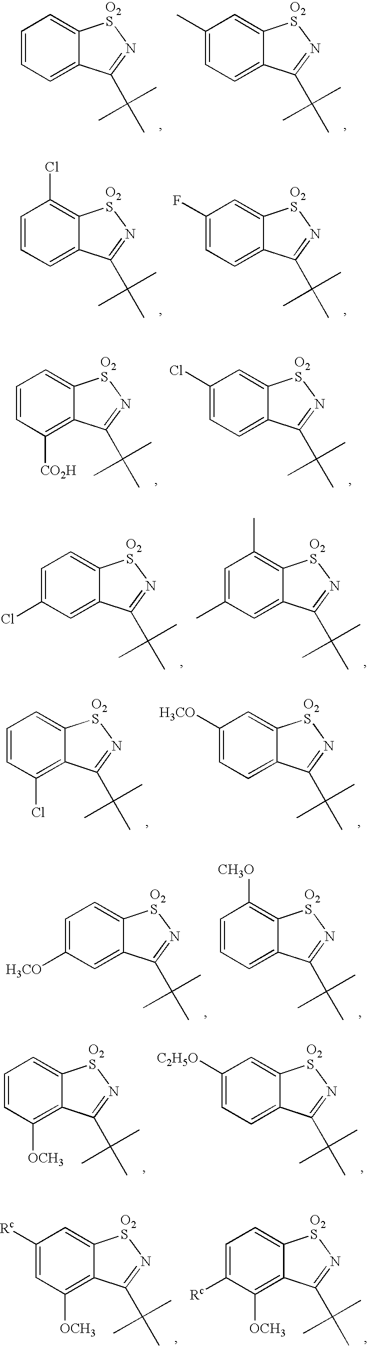

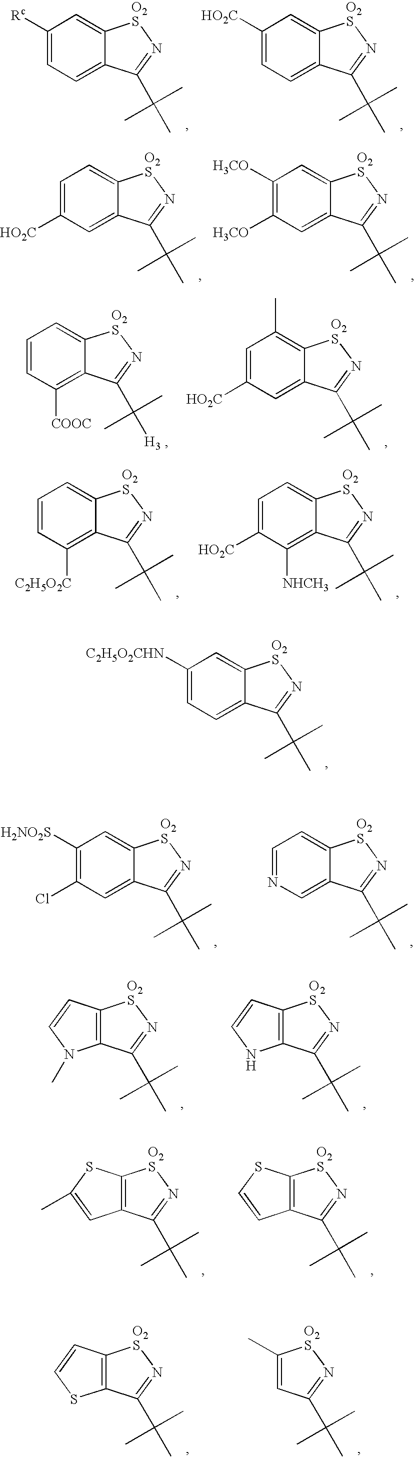

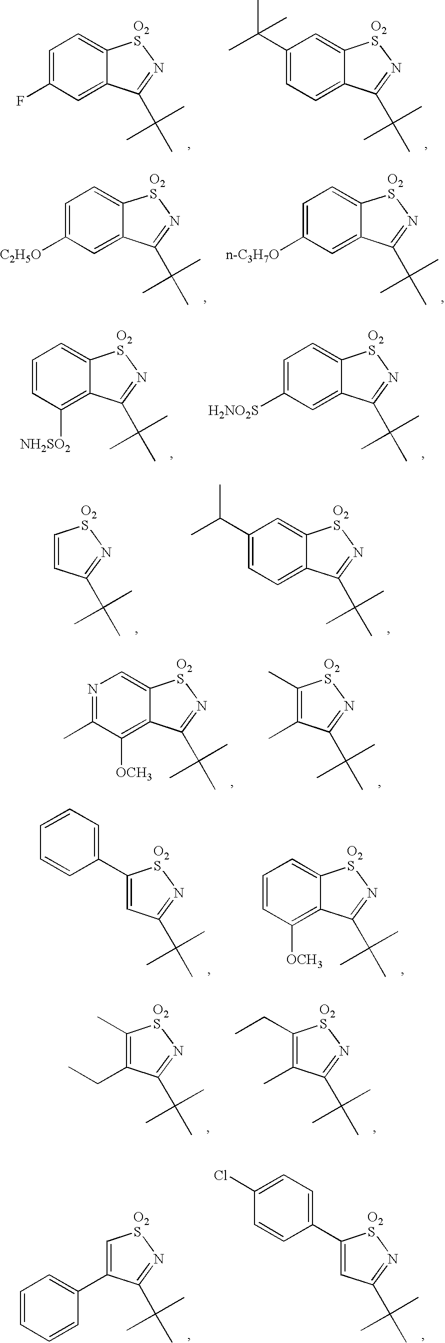

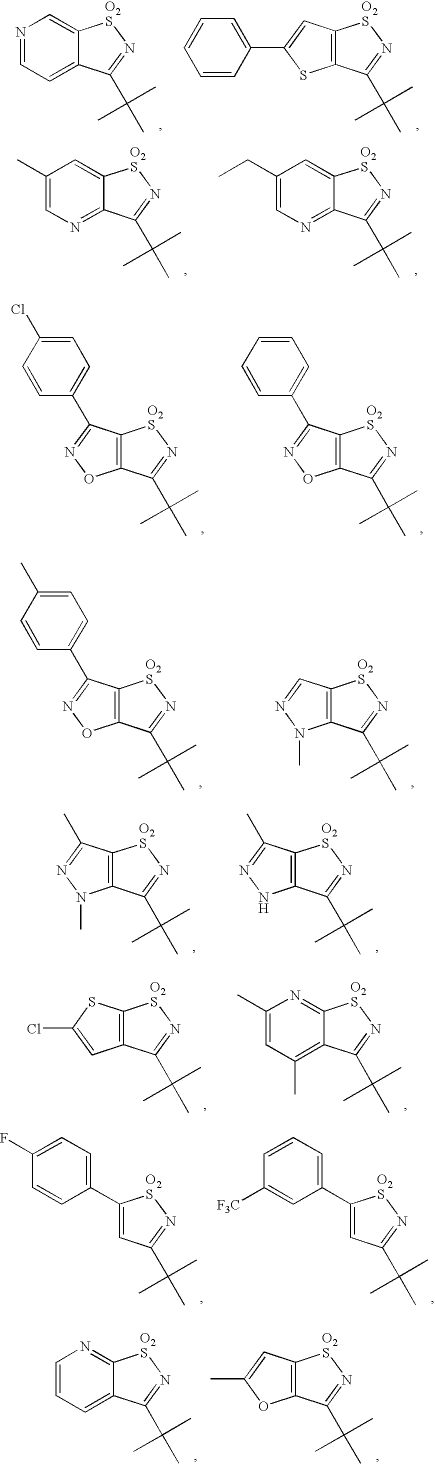

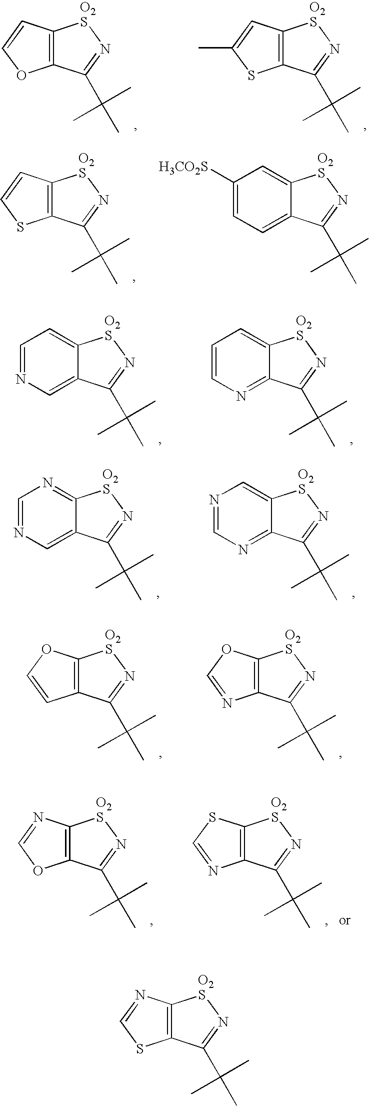

where R^{c} is amino, methylsulfonylamino, ethylsulfonylamino, methylamino, dimethylamino, acetylamino, methoxy, ethoxy, methylaminocarbonyl, aminocarbonyl, diethylaminocarbonyl, dimethylaminocarbonyl, or ethoxycarbonylamino.

Within the above preferred and more preferred groups in (7), a particularly preferred group of compounds is that where R² is hydrogen.

(6) Within the above preferred, more preferred and even more preferred groups, yet another particularly preferred group of compounds is that wherein R³ and R⁴ together with the atoms to which they are attached form a group selected from:

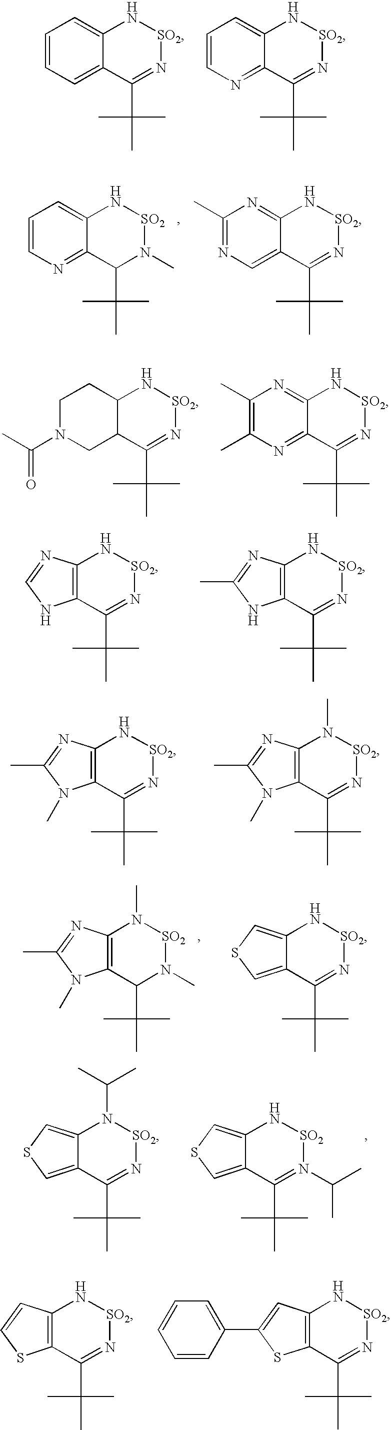

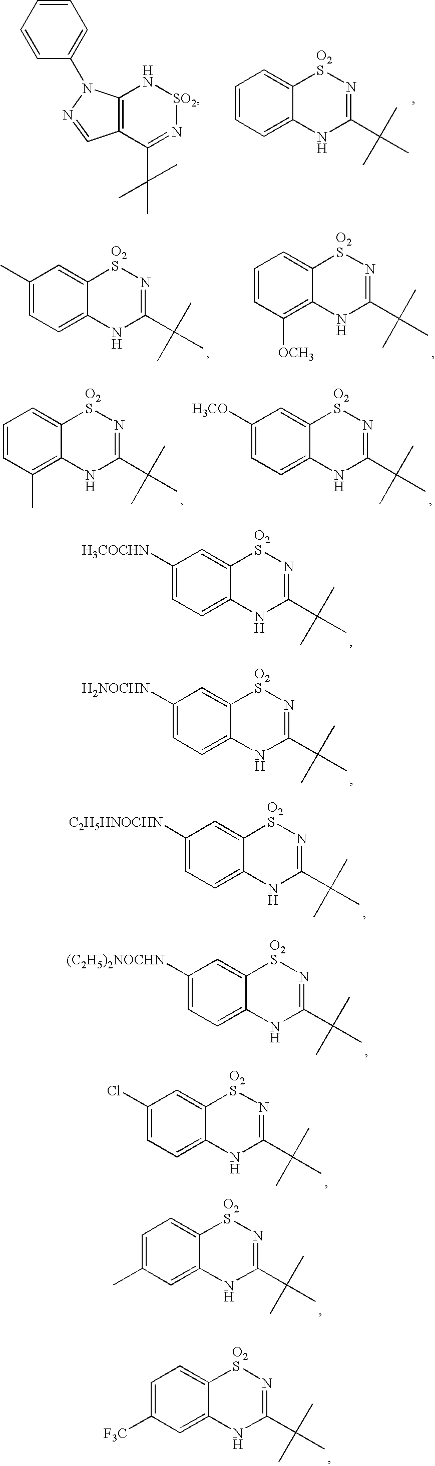

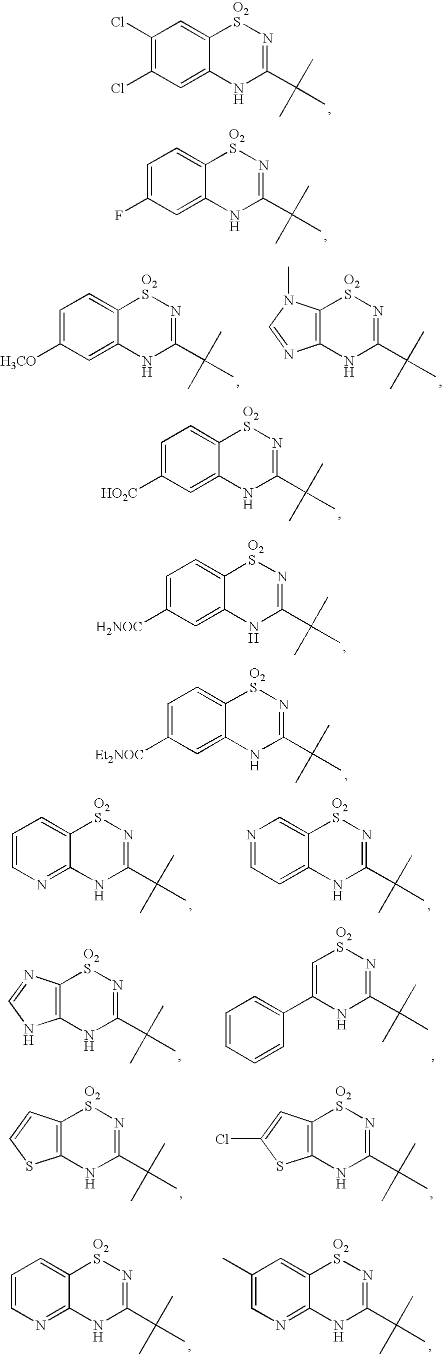

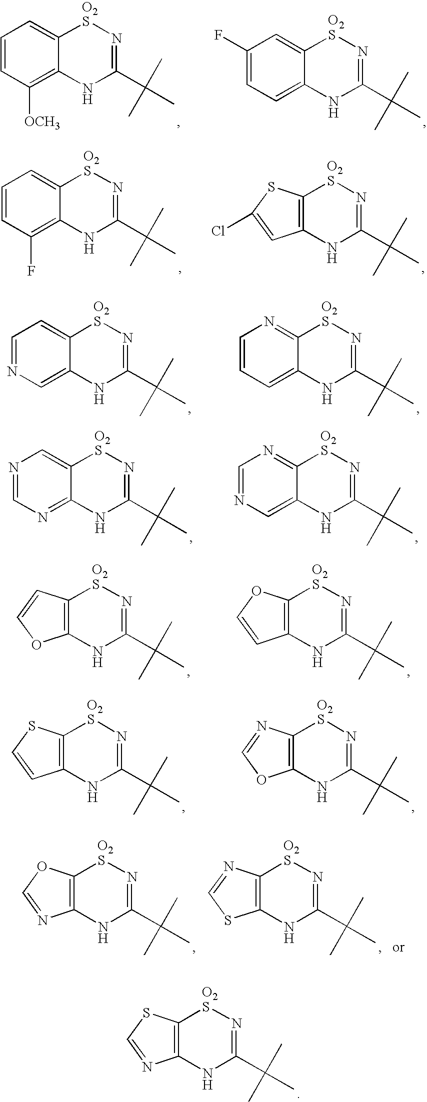

In the above groups, the hydrogen atom attached to the nitrogen can be replaced by alkyl haloalkyl, preferably, methyl, ethyl, propyl, isopropyl, n-, iso-, or tert-butyl, or trifluoromethyl), methylsulfonylmethyl, methoxycarbonylmethyl, 2-methylsulfonylethyl, 2-methoxycarbonylethyl, 2-methylpropyl, 2,2-dim ethylpropyl, 1-methylpiperidin-4-yl, 1-methylpiperidin-4-ylmethyl, 2-(1-methylpiperazin-4-yl)ethyl, 2-dimethylaminoethyl, 3-dimethylaminopropyl, 2-pyridin-4-ylethyl, 3-pyrrolidin-1-ylpropyl, 3-piperidin-1-ylpropyl, 2,2,2-trifluoroethyl, or 2-morpholin-4-ylethyl.

Within the above preferred and more preferred groups in (6 and 7), a particularly preferred group of compounds is that where R² is hydrogen.

A person skilled in the art will recognize that compounds of Formula (Ia) where R² is hydrogen can tautomerize to give a compound of Formula (Ib) where R⁴ or R^{4a} is hydrogen and compounds of Formula (Ib) where R⁴ or R^{4a} is hydrogen can tautomerize to give a compound of Formula (Ia) or (Ib′) respectively, where R² is hydrogen. Such tautomers are within the scope of this group. The amount of each tautomer present will depend on various conditions such as steric hinderance, pH, temperature, and the like. Accordingly, this group encompasses individual tautomeric forms of compounds of Formula (Ia) as well as mixtures thereof.

(II). Another preferred group of compounds is represented by Formula (II).

Within this group (II), a more preferred group of compounds is that wherein E and R^{1a} are as defined in preferred group (I) above, R¹ and R² are hydrogen and Z is —CO—.

Within this group of compounds, a more preferred group of compounds is that wherein Q is —CO—.

Within this group of compounds, another more preferred group of compounds is that wherein Q is —OCO—.

Within this group of compounds, yet another more preferred group of compounds is that wherein Q is —NHCO—.

Within the preferred and more preferred groups, an even more preferred group of compounds is that wherein R^{3c} is alkyl, -alkylene-C(O)OR⁴⁰, cycloalkyl, cycloalkylalkyl, heterocycloalkyl, heterocycloalkylalkyl, aryl, aralkyl, heteroaryl or heteroaralkyl wherein said cycloalkyl, heterocycloalkyl, aryl or heteroaryl ring is optionally substituted with one or two R^{k}.

Within the above preferred, more preferred groups, a particularly preferred group of compounds is that wherein R^{3c}-Q- is a group selected from acetyl, azetidin-3-ylcarbonyl, benzyloxycarbonyl, 1-benzyloxycarbonylpiperidin-4-ylcarbonyl, benzylsulfonyl, bicyclo[2.2.2]hept-2-ylcarbonyl, bicyclo[2.2.1]hept-2-ylcarbonyl, tert-butoxycarbonyl, carboxyacetyl, 2-carboxypropionyl, 3-carboxypropionyl, 2-cyclohexylacetyl, 4-cyclohexylbutyryl, 2-cyclohexylethylsulfonyl, cyclohexylmethoxycarbonyl, 3-cyclohexylpropionyl, 2-cyclopentylethylsulfonyl, 3-cyclopentylpropionyl, dimethylcarbamoyl, 6-hydroxypyrid-3-ylcarbonyl, 1H-imidazol-4-ylcarbonyl, methoxycarbonyl, methylsulfonyl, 4-methylvaleryl, morpholin-4-ylcarbonyl, 2-morpholin-4-ylethylcarbonyl, naphth-1-ylmethylcarbonyl, 3-phenylpropionyl, piperazin-1-ylcarbonyl, piperidin-4-ylcarbonyl, pyrazin-2-ylcarbonyl, pyridin-3-ylcarbonyl, pyridin-4-ylcarbonyl, pyridin-3-ylaminocarbonyl, tetrahydropyran-4-ylcarbonyl, and tetrahydropyran-4-yloxycarbonyl.

R^{3c}-Q- particularly represents acetyl, benzoyl, benzyloxycarbonyl, benzylsulfonyl, bicyclo[2.2.2]hept-2-ylcarbonyl, tert-butoxycarbonyl, tert-butyryl, 4-tert-butoxycarbonylpiperazin-1-ylcarbonyl, 1-tert-butoxycarbonylpiperidin-4-ylcarbonyl, 2-cyclohexylacetyl, 4-cyclohexylbutyryl, 2-cyclohexylethylsulfonyl, 3-cyclohexylpropionyl, 2-cyclopentylethylsulfonyl, 4-methylpiperazin-1-ylcarbonyl, methylsulfonyl, 4-methylvaleryl, 3-morpholin-4-ylpropionyl, naphth-2-ylmethyl, 3-phenylpropionyl, piperazin-1-ylcarbonyl, piperidin-4-ylcarbonyl or pyridin-3-ylcarbonyl, wherein within R^{3c} any alicyclic or aromatic ring system present may be substituted further by 1 to 3 radicals independently selected from 3-aminomethyl and 3-tert-butoxycarbonylaminomethyl.

R^{3c}-Q- especially represents morpholin-4-ylcarbonyl, methoxycarbonyl, methylsulfonyl, piperidin-4-ylcarbonyl, pyrazin-2-ylcarbonyl pyridin-3-ylcarbonyl, pyridin-4-ylcarbonyl, tetrahydropyran-4-ylcarbonyl or tetrahydropyran-4-yloxycarbonyl.

(III). Another preferred group of compounds is represented by Formula (III).

Within this group (III), a more preferred group of compounds is that wherein E is as defined in preferred group (I) above.

Within this preferred group of compounds, a more preferred group of compounds is that wherein:

R^{3d} and R^{3e} are independently -(alkylene)-X⁹—R⁴³ wherein X⁹ is bond, —S—, —O—, —C(O)—, —CONR⁴⁴—, —NR¹⁴SO₂—, or —SO₂— where R⁴⁴ is hydrogen or alkyl and R⁴³ is alkyl, haloalkyl, cycloalkyl, cycloalkylalkyl, aryl, aralkyl, heteroaryl, heteroaralkyl, heterocycloalkyl, or heterocycloalkylalkyl wherein the aromatic or alicyclic rings in R^{3d} and R^{3e} are optionally substituted with one, two or three R^{m} independently selected from alkyl, cyano, halo, haloalkyl, haloalkoxy, alkylcarbamoyloxy, hydroxy, alkoxy, carboxy, alkoxycarbonyl, acyl, carbamoyl, alkylsulfonylamino, and alkylsulfonyl, and one R^{m} selected from aryl, aralkyl, heteroaryl, heteroaralkyl, cycloalkyl, cycloalkylalkyl, heterocycloalkyl, or heterocycloalkylalkyl optionally substituted with one to three Rⁿ.

Preferably, R^{3d} and R^{3e} are independently benzylcarbamoyl-methyl, benzyl, benzylsulfanylmethyl, 2-benzenesulfonylethyl, benzenesulfonylmethyl, 2-benzo[1,3]dioxol-5-yl-2-oxo-ethyl, 2-benzo[b]thiophen-2-yl-2-oxo-ethyl, biphenyl-2-ylmethylsulfonylmethyl, biphenyl-4-ylmethyl-sulfonylmethyl, biphenyl-3-ylmethyl, biphenyl-4-ylmethyl, 2-biphenyl-4-yl-2-oxo-ethyl, 3,5-bis-trifluoromethylbenzylsulfonylmethyl, 3-bromo-benzyl, 2-oxo-2-pyrrolidin-1-yl-ethyl, 2-bromobenzylsulfonylmethyl, (butylmethylcarbamoyl)methyl, 4-tert-butyl-benzylsulfonylmethyl, (3-carbamoylphenylcarbamoyl)methyl, (4-carbamoylphenylcarbamoyl)methyl, 4-carboxybenzylsulfonylmethyl, 2-(3-chloro-benzo[b]thiophen-2-yl)-2-oxo-ethyl, 2-(4′-chlorobiphenyl-4-yl)-2-oxo-ethyl, 3-chloro-2-fluoro-benzylsulfonylmethyl, 2-chlorobenzylsulfonylmethyl, 3-chlorobenzylsulfonylmethyl, 4-chlorobenzylsulfonylmethyl, 2-(4-chlorophenyl)-2-oxo-ethyl, 5-chlorothiophen-2-ylmethylsulfonylmethyl, 2-(3-chlorothiophen-2-yl)-2-oxo-ethyl, 2-chloro-5-trifluoromethylbenzylsulfonylmethyl, cyclohexylcarbamoylmethyl, 2-cyclohexylethanesulfonylmethyl, cyclohexylmethylsulfonylmethyl, 2-cyclohexylethyl, cyclohexylmethyl, 2-cyanobenzylsulfonylmethyl, cyclopropylmethylsulfonylmethyl, 3-cyanobenzylsulfonylmethyl, 4-cyanobenzylsulfonylmethyl, 2,5-dichlorobenzylsulfonylmethyl, 2,6-dichlorobenzylsulfonylmethyl, 3,4-dichlorobenzylsulfonylmethyl, 2-[2-(1,1-difluoromethoxy)-benzenesulfonyl]-ethyl, 2-[3-(1,1-difluoromethoxy)benzenesulfonyl]ethyl, 2-[4-(1,1-difluoromethoxy)-benzenesulfonyl]-ethyl, 2-(1,1-difluoromethoxy)-benzylsulfonylmethyl, 3-(1,1-difluoromethoxy)-benzylsulfonylmethyl, 4-(1,1-difluoromethoxy)-benzylsulfonylmethyl, 2,3-difluoro-benzylsulfonylmethyl, 2,4-difluoro-benzylsulfonylmethyl, 2,5-difluoro-benzylsulfonylmethyl, 2,6-difluorobenzylsulfonylmethyl, 3,4-difluorobenzylsulfonylmethyl, 3,4-dichlorobenzyl-sulfonylmethyl, 2-(3,4-difluorophenyl)-2-oxo-ethyl, 2-(3,4-dimethoxyphenyl)-2-oxo-ethyl, 4-dimethylcarbamoylmethyl, 3,5-dimethylisoxazol-4-ylmethyl-sulfonylmethyl, 3,5-dimethylbenzylsulfonylmethyl, 2-(3-fluoro-4-methoxyphenyl)-2-oxo-ethyl, 2-fluoro-3-methylbenzylsulfonylmethyl, 2-fluorobenzylsulfonylmethyl, 3-fluorobenzylsulfonylmethyl, 4-fluorobenzylsulfonylmethyl, 2-(4-fluorophenyl)-2-oxo-ethyl, 4-fluoro-2-trifluoromethoxybenzylsulfonylmethyl, 2-fluoro-3-trifluoromethylbenzylsulfonylmethyl, 2-fluoro-4-trifluoromethylphenylmethylsulfonylmethyl, 2-fluoro-5-trifluoromethylbenzyl-sulfonylmethyl, 2-fluoro-6-trifluoromethyl-benzylsulfonylmethyl, 4-fluoro-3-trifluoromethylbenzylsulfonylmethyl, 2-(4-hydroxyphenyl)-2-oxo-ethyl, isobutylsulfanylmethyl, isopropylcarbamoylmethyl, 2-(4-methylsulfonylamino-phenyl)-2-oxo-ethyl, 2-(4-methylsulfonyl-piperazin-1-yl)-2-oxo-ethyl, 5-methyl-2-oxo-hexyl, 2-methoxybenzylsulfonylmethyl, 4-methoxybenzylsulfonylmethyl, 2-(4-methoxyphenyl)-2-oxo-ethyl, 3-methylbenzylsulfonylmethyl, 2-methylpropane-1-sulfonylmethyl, 2-(5-methylthiophen-2-yl)-2-oxo-ethyl, 2-methylthiazol-4-yl-methylsulfonylmethyl 5-methylthiophene-2-sulfonylmethyl, naphthalen-2-ylmethylsulfonylmethyl, 2-naphthalen-2-yl-2-oxo-ethyl, naphthalene-2-sulfonylmethyl, 2-morpholin-4-yl-2-oxo-ethyl, 2-oxo-2-piperidin-1-ylethyl, 2-oxo-2-phenylethyl, 2-oxo-2-pyrrolidin-1-ylethyl, 2-oxo-2-thiophen-2-ylethyl, 2-oxo-2-thiophen-3-ylethyl, 2-oxo-2-p-tolylethyl, 2-oxo-2-(4-trifluoromethoxyphenyl)-ethyl, 1-oxy-pyridin-2-ylmethylsulfonylmethyl, phenylcarbamoylmethyl, 2-benzylsulfonylethyl, 4-benzylsulfonylmethyl, 2-phenylsulfanylethyl, pyridin-3-ylcarbamoylmethyl, pyridin-4-ylcarbamoylmethyl, 2-(pyridin-2-sulfonyl)-ethyl, 2-(pyridin-4-sulfonyl)-ethyl, pyridin-2-ylmethylsulfonylmethyl, pyridin-3-ylmethylsulfonylmethyl, pyridin-4-ylmethylsulfonylmethyl, tetrahydropyran-4-yloxymethyl, thiophene-2-sulfonylmethyl, o-tolylmethylsulfonylmethyl, m-tolylmethylsulfonylmethyl, p-tolylmethylsulfonylmethyl, 2-(2-trifluoromethoxybenzenesulfonyl)-ethyl, 2-(3-trifluoromethoxy-benzenesulfonyl)-ethyl, 2-(4-trifluoromethoxy-benzenesulfonyl)-ethyl, 2-trifluoromethoxy-benzylsulfanylmethyl, 2-trifluoromethoxy-benzylsulfonylmethyl, 3-trifluoromethoxy-benzylsulfonylmethyl, 4-trifluoromethoxy-benzylsulfonylmethyl, 2-trifluoromethyl-benzylsulfanylmethyl, 2-trifluoromethyl-benzylsulfonylmethyl, 3-trifluoromethyl-benzylsulfonylmethyl, 4-trifluoromethyl-benzylsulfonylmethyl, 2,3,4-trifluoro-benzylsulfonylmethyl, 2,3,5-trifluoro-benzylsulfonylmethyl, 2,4,5-trifluoro-benzylsulfonylmethyl, 2,4,6-trifluoro-benzylsulfonylmethyl and 2,5,6-trifluoro-benzylsulfonylmethyl. Preferred R^{3d} and R^{3e} groups are benzylsulfanylmethyl, 3-cyano-benzyl-sulfonylmethyl, cyclohexylmethyl, 2-difluoromethoxy-benzylsulfonylmethyl, isobutylsulfanylmethyl, (2-methyl-thiazol-4-yl)-methylsulfonylmethyl, 2-morpholin-4-yl-2-oxo-ethyl, 2-oxo-2-piperidin-1-yl-ethyl, 2-oxo-2-pyrrolidin-1-yl-ethyl, benzylsulfonylmethyl, tetrahydropyran-4-yloxymethyl, and 3-trifluoromethyl-benzylsulfonylmethyl. Particularly preferred R^{3d} and R^{3e} groups are benzylsulfanylmethyl, 2-difluoromethoxy-benzylsulfonylmethyl, 2-morpholin-4-yl-2-oxo-ethyl and benzylsulfonylmethyl.

(IV). Another preferred group of compounds is represented by Formula (IV).

Within this group (IV), a more preferred group of compounds is that wherein E and R^{1a} are as defined in preferred group (I) above.

Within this preferred group, a more preferred group of compounds is that wherein R^{3g} is —OH or —OC(O)NR⁴⁹R⁵⁰, preferably wherein R⁴⁹ is hydrogen, alkyl, hydroxyalkyl, alkoxyalkyl, aryl, aralkyl, cycloalkyl, cycloalkylalkyl, heteroaryl, heteroaralkyl, heterocycloalkyl or heterocycloalkylalkyl and R⁵⁰ is hydrogen or alkyl or R⁴⁹ and R⁵⁰ together with the nitrogen atom to which both R⁴⁹ and R⁵⁰ attached form a heterocycloalkyl ring, which ring may be unsubstituted or substituted with hydroxy, alkylsulfonyl, alkoxycarbonyl, aralkyl, or acyl. Preferably, R^{3g} is selected from —OH, dimethylcarbamoyloxy, morpholin-4-ylcarbonyloxy, piperidin-1-yl-carbonyloxy, pyrrolidin-1-yl-carbonyloxy, 4-tert-butoxycarbonylpiperazin-1-ylcarbonyloxy, N-benzyl-carbamoyloxy, pyrrolidin-1-yl-carbonyloxy, piperidin-1-yl-carbonyloxy, 4-methanesulfonyl-piperazin-1-yl-carbonyloxy, 4-ethoxycarbonylpiperazin-1-ylcarbonyloxy, N-cyclohexyl-carbamoyloxy, N-phenyl-carbamoyloxy, N-butyl-N-methyl-carbamoyloxy, N-pyridin-3-yl-carbamoyloxy, N-isopropyl-carbamoyloxy, N-pyridin-4-yl-carbamoyloxy, N-phenethyl-carbamoyloxy, piperazinecarbonyloxy, N-naphthalen-2-yl-carbamoyloxy, 4-benzyl-piperazin 1-ylcarbamoyloxy, 4-(1-furan-2-yl-carbonyl)-piperazin-1-ylcarbamoyloxy, thiomorpholin-4-ylcarbonyloxy, 1,1-dioxo-1λ⁶-thiomorpholin-4-ylcarbonyloxy, morpholin-4-ylcarbonyloxy, 2-methoxyethylcarbamoyloxy, diethylcarbamoyloxy, 2-hydroxyethylcarbamoyloxy, tetrahydrofuran-2-ylmethylcarbamoyloxy, cyclopropylcarbamoyloxy, tert-butylcarbamoyloxy, 3-hydroxypyrrolidin-1-ylcarbonyloxy and carbamoyloxy. More particularly, R^{3g} is morpholin-4-ylcarbonyloxy, 2-methoxyethylcarbamoyloxy, diethylcarbamoyloxy, pyrrolidin-1-ylcarbonyloxy, 2-hydroxyethylcarbamoyloxy, tetrahydro-furan-2-ylmethylcarbamoyloxy, cyclopropylcarbamoyloxy, tert-butylcarbamoyloxy, 3-hydroxy-pyrrolidin-1-yl-carbonyloxy or carbamoyloxy.

Another more preferred group of compounds within group (IV) is that wherein R^{3g} is —NHR⁴⁸ wherein R⁴⁸ is aryl or heteroaryl or —NR⁴⁷R⁴⁸ wherein R⁴⁷ is heterocycloalkyl and R⁴⁸ is hydrogen or alkoxyalkyl, or R⁴⁷ and R⁴⁸ independently are aralkyl or heteroaralkyl, wherein within R⁴⁷ and R⁴⁸ any alicyclic or aromatic ring system is optionally substituted with one, two, or three R^{q} independently selected from alkyl, cyano, halo, nitro, haloalkyl, hydroxy, hydroxyalkyl, alkoxy, alkoxyalkyl, carboxyl, alkoxycarbonyl, acyl, carbamoyl, or alkylsulfonylamino. Preferably, R^{3g} is selected from 5-nitrothiazol-2-ylamino, 2-nitrophenylamino, pyrimidin-2-ylamino, tetrahydropyran-4-ylamino, N-(2-methoxyethyl)-N-(tetrahydropyran-4-yl)-amino, 1-methyl-piperidin-4-ylamino, isopropylamino, di(thien-2-ylmethyl)amino or di(benzyl)amino.

Yet another more preferred group of compounds within group (IV) is that wherein R^{3g} is —OR⁴⁶ wherein R⁴⁶ is 4-methoxy-phenyl, 4-hydroxymethyl-phenyl, methoxymethyl, phenyl-methanoyl, 1-(4-phenoxy-phenyl)-methanoyl, 3-biphenyl, 4-biphenyl, 1-biphenyl-4-yl-methanoyl, naphthalen-2-yl-methanoyl, benzo[1,3]dioxol-5-yl-methanoyl, (4-methanesulfonylaminophenyl)-methanoyl, benzo[b]thien-2-yl-methanoyl, 4′-chloro-4-biphenyl, 4-hydroxyphenylmethanoyl, 3-chloro-benzo[b]thien-2-yl-methanoyl, thien-2-yl-methanoyl, thien-3-ylmethanoyl, 3-chlorothien-2-yl-methanoyl, 5-methylthien-2-ylmethanoyl, 4-methoxyphenylmethanoyl, 4-trifluoromethoxy-phenylmethanoyl, 4-chlorophenylmethanoyl, 3-bromophenyl, cyclohexylmethyl, 3,4-dimethoxyphenylmethanoyl, 3,4-difluorophenylmethanoyl, 3-fluoro-4-methoxyphenylmethanoyl, 4-fluorophenylmethanoyl, 4-trifluoromethylphenylmethanoyl, 4-formylphenylformyl, 3-formylphenylformyl, 4-methylpentanoyl, tetrahydropyran-4-ylmethyl, or 2-morpholin-4-yl-2-oxo-ethyl.

Most particularly preferred are compounds of the invention where R^{3g} is selected from —OH, dimethylcarbamoyloxy, morpholin-4-ylcarbonyloxy, piperidin-1-yl-carbonyloxy, pyrrolidin-1-yl-carbonyloxy, pyrimidin-2-ylamino, tetrahydro-pyran-4-ylamino, 1-methyl-piperidin-4-yl-amino, N-(2-methoxyethyl)-N-(tetrahydro-pyran-4-yl)amino, isopropylamino, and cyclohexylamino.

Additionally, in the preferred embodiments above, a number of different preferences have been given above, and following any one of these preferences results in a compound of this invention that is more presently preferred than a compound in which that particular preference is not followed. However, these preferences are generally independent; and following more than one of these preferences may result in a more presently preferred compound than one in which fewer of the preferences are followed.

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991. Compounds of the present invention can be prepared by the procedures described in Schemes 1-10 below.

Compounds of Formula (Ia) where E is —C(R⁵)(R⁶)X¹ or —C(R^{5a})(R^{6a})CN, R² is hydrogen and X¹, R¹, R^{1a}, R³, R⁴, R⁵, R^{5a}, R⁶ and R^{6a} are as defined in the Summary of the Invention can be prepared as shown in Scheme 1 below.

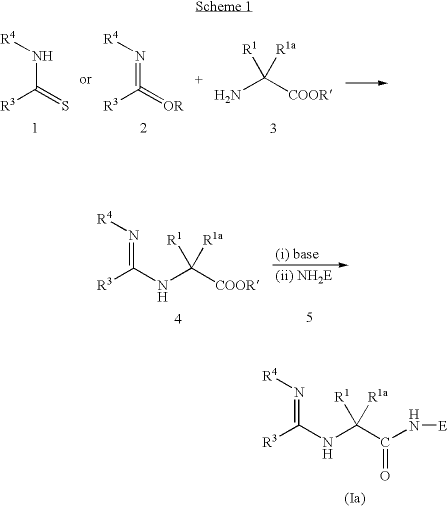

Compounds of Formula (Ia) can be prepared by reacting an amino acid derivative of formula 3 where R′ is alkyl with a thione of formula 1 to give a compound of formula 4. The reaction is carried out in the presence of a suitable coupling agent such as 2-chloro-1-methylpyridinium iodide (Yong, Y. F, et. al., J. Org. Chem. 1997, 62, 1540), phosgene or triphosgene (Barton, D. H., et. al., J. Chem. Soc. Perkin Trans. I, 1982, 2085), alkyl halides (Brand, E and Brand, F. C., Org. Synth., 1955, 3, 440), or carbodiimide (Poss, M. A., et. al., Tet. Lett., 1992, 40, 5933).

Alternatively, a compound of formula 4 is prepared by reacting a hydroxy compound of formula 2 with an amino acid derivative of formula 3. The reaction is carried out optionally in the presence of a base such as triethylamine. Suitable reaction conditions are known to those skilled in the art e.g., see Haake, M., et. al., Synthesis, 1991, 9, 753; Dauwe, C., et al, Synthesis, 1995, 2, 171, Reid, et. al., Justus Liebigs Ann. Chem., 1966, 97, 696; and Dean N. D., and Papadopoulos, E. P. J. Het. Chem., 1982, 19, 1117.

Compounds of formula 1 are commercially available or they can be prepared by methods well known in the art. For example, N-phenyl-2,2,2-trifluorothioacetamide can be prepared by method described in Tet. Lett., 2001, 42, 46, 8181-8184; N-thiazol-2-ylthioacetamide can be prepared by the method described in Chem. Heterocyclo, 1972, 848-851; and N-thiazol-2-ylphenylthiobenzamide can be prepared by the method described in Chem. Heterocyclo, 1988, 337-344. Other compounds of formula 1 can be prepared by methods described in PCT Application Publication No. WO 02/20485 the disclosure of which is incorporated herein by reference in its entirety. Compounds of formula 2 are either commercially available or they can be prepared by methods known in the art. Some such methods are described in Francesconi, I., et. al., J. Med. Chem., 1999, 42, 2260; Kurzer, F., et. al., Org. Synth. 1963, 645; and Futmnan, A. D., U.S. Pat. No. 3,984,410. For example, ethyl benzenesulfonyl formimidate can be prepared by methods described in Stetter, H. and Theisen, D. H. Chem Ber., 1969, 102, 1641-42 and Ortiz, J. A., Arzneim.-Forsch./Drug Res, 1977, 47,431434.

Amino acids of formula 3 such as esters of alanine, cysteine, aspartic acid, glutamic acid, phenylalanine, histidine, and lysine are commercially available. Others can be prepared by methods well known in the art. Some such methods are described in PCT Applications Publication Nos. WO 00/55144, WO 01/19816, WO 02/20485, WO 03/029200, U.S. Provisional Application No. 60/422,337, U.S. Pat. Nos. 6,353,017B1, 6,492,662B1, 353,017 B1 and 6,525,036B 1, the disclosures of which are incorporated herein by reference in their entirety.

Hydrolysis of the ester group in 4, followed by reaction of the resulting acid with an amine of formula 5 where E is as defined in the Summary of the Invention provides a compound of Formula (Ia). The reaction is carried out in the presence of a suitable coupling agent (e.g., benzotriazol-1-yloxy-trispyrrolidinophosphonium hexafluorophosphate (PyBOP®), 1-(3-dimethylaminopropyl)-3-ethylcarbodiimide hydrochloride (EDCI), O-(7-azabenzotrizol-1-yl)-1,1,3,3, tetra-methyluronium-hexafluorophosphate (HATU), O-benzotriazol-1-yl-N,N,N′,N′-tetramethyl-uronium hexafluorophosphate (HBTU), 1,3-dicyclohexylcarbodiimide (DCC), or the like) and optionally an appropriate catalyst (e.g., 1-hydroxybenzotriazole (HOBt), 1-hydroxy-7-azabenzotriazole (HOAt), or the like) and non-nucleophilic base (e.g., triethylamine, N-methylmorpholine, and the like, or any suitable combination thereof) at ambient temperature and requires 5 to 10 h to complete. Suitable reaction solvents include, but are not limited to, dimethylformamide, methylene chloride, and the like.

Alternatively, the free acid of compound 4 can be converted to an acid halide and then reacted with 5 to give a compound of Formula (Ia). The reacting is carried out in the presence of a base such as triethylamine, pyridine, and the like and in a suitable organic solvent such as tetrahydrofuran, dioxane, and the like. Compounds of formula 5 are either commercially available or they can be prepared by methods well known in the art. Some such methods are disclosed in working examples below. Other methods are disclosed in U.S. patent application Ser. Nos. 60/373,176, 09/525,507, and Ser. No. 10/035,783 the disclosures of which are incorporated herein by reference in their entirety.

A compound of Formula (Ia) can be converted to other compounds of Formula (Ia). For example, a compound of Formula (Ia) where E is —C(R⁵)(R⁶)C(R⁷)(R⁸)R¹⁰ where R⁷ is hydrogen and R⁸ is hydroxy can be converted to other compounds of Formula (Ia) where E is —C(R⁵)(R⁶)COR¹⁰ by oxidation of the hydroxy group. The oxidation reaction is carried out with an oxidizing agent (e.g., Dess-Martin Periodinane®, TEMPO/bleach, or the like) in a suitable solvent (e.g., acetonitrile, dichloromethane, methanol, water, or the like, or any suitable combination thereof) at ambient temperature and requires 16 to 24 h to complete.

Alternatively, a Formula (Ia) where E is —C(R⁵)(R⁶)X¹ or —C(R^{5a})(R^{6a})CN, R² is hydrogen, and R¹, R^{1a}, R³, R⁴, R⁵, R^{5a}, R⁶ and R^{6a} are as defined in the Summary of the Invention can be prepared by proceeding as illustrated and described in Scheme 2 below.

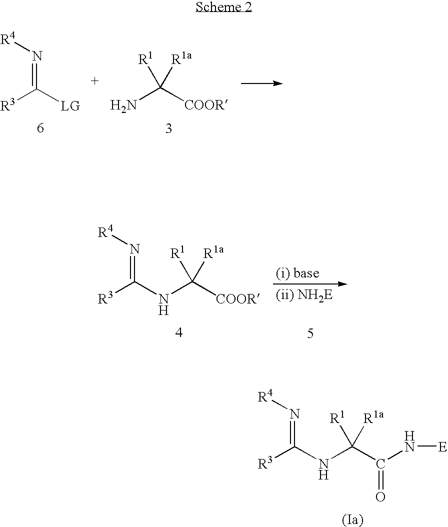

Reaction of a compound of formula 6 where LG is a leaving group such as halo with an amino acid derivative of formula 3 provides a compound of formula 4. The reaction is carried out by methods well known in the art. Some such methods are described in Dunn. A. D., Org. Prep. Proceed Int., 1998, 30, 709; Lindstroem, S., et. al., Heterocycles, 1994, 38, 529; Katrizky, A. R., et. al., Synthesis, 1990, 561; Hontz, A. C., et. al., Org. Synth., 1963, IV, 383; and Stephen, H., J. Chem., Soc., 1957, 490. Compound 4 is then converted to a compound of Formula (Ia) as described in Scheme 1 above.

Compounds of formula 6 are either commercially available or they can be readily prepared by methods well known in the art. For example, 3-chloro-1,1-dioxobenzo[d]isothiazole is commercially available. 4-Chlorobenzo[e][1,3]oxazin-2-one can be obtained by treating benzo[e][1,3]oxazine-2,4-dione with phosphorus pentachloride in refluxing toluene. Similarly, 3-chloro-2,3-dihydro-thieno[3,4-d]isothiazole 1,1-dioxide and 3-chloro-2,3-dihydrothieno[3,2-d]isothiazole can be prepared from 1,1-dioxide 1,1-dioxo-1,2-dihydro-1λ⁶-thieno[3,4-d]isothiazol-3-one and 1,1-dioxo-1,2-dihydro-1λ⁶-thieno[3,2-d]isothiazol-3-one respectively, as described above. 1,1-Dioxide1,1-dioxo-1,2-dihydro-1λ⁶-thieno[3,4-d]isothiazol-3-one and 1,1-dioxo-1,2-dihydro-1λ⁶-thieno[3,2-d]isothiazol-3-one can be prepared by the procedures described in J. Org. Chem., 1980, 45, 617-620. Other compounds of formula 6 disclosed in preferred embodiment group (I) (7) and (8) above, can be prepared from corresponding carbonyl compounds known in the art by converting them to the corresponding halo derivative as described above. The following references describe the synthesis of some of the carbonyl starting materials: Chem. Ber. 1962, 84, 509; Cohen, E. Klarberg, B, JACS, 1962, 84, 1992-2002; BASF patent, FR 2276309, DE 2430353; Edenhofer, A., Meister, W., Helv. Chim. Acta, 1977, 60, 521-523; Kloek, J. A.; Leschinsky, K. L., J. Org. Chem., 1978, 43, 3824-3827; Goya, et al., J. Heterocycl. Chem, 1984, 21, 861-864; Meyer, R. B.; and Skibo, E. B., J. Med. Chem., 1979, 22, 944-948; Ochoa, C; and Stud, M., J. Heterocycl. Chem., 1978, 15, 221-224; Edenhofer, A., and Meister, W., Helv. Chim. Acta, 1977, 60, 521-523; Goya, et al, Synthesis, 1989, 280-282; Monsanto patent, U.S. Pat. No. 4,139,700; Womhoff, H. and Ertas, M., Synthesis, 1985, 190-194; Raffa, Farmaco Ed. Sci., 1957, 12, 41-47; Girare, Y. et al., J. Chem. Soc. Perkin Trans. 1, 1979, 1043-1047; Parke, W., J. Chem. Soc., 1950, 1760, 1763; Raffa, Farmaco Ed. Sci., 1960, 15, 716-725; Raffa, Farmaco Ed. Sci., 1966, 21, 16-29; Hayman, D. F., et al, J. Pharm. Pharmacol., 1962, 14, 522-533; Raffa, Farmaco Ed. Sci., 1962, 17, 234-243; Blicke, F. F. and Lee, C. M., J. Org. Chem., 1961, 26, 1861-1867; Kotovskaya, S. K., et al., J. Pharm. Chem., 1979, 13, 4, 389-392; Ofiserov, V. I, et al., Chem. HeterocycL Compd., 1976, 12, 924-927; Thompson, M. E., Synthesis, 1988, 9, 733-735; Arranz, M. E., et al., Heterocycles, 1977, 45, 9, 1767-1774; Neill, C. G, et al., Tetrahedron, 1988, 54, 44, 13645-13654; Phillips, D., et al., Bioorg. Med. Chem., 2002, 10, 5, 1229-1248; Ihara Chem. Ind. Patent FR 231,4185; DE 261,6611, Becke, F. and Hagen, H., Justus Liebigs Ann. Chem., 1969, 729, 146-151; Burri, K. F., Helv. Chim. Acta., 1990, 73, 1, 69-80; Kwon, Soon-Kyoung and Park, Myung-Sook, Arzneim. Forsch., 1996, 46, 10, 966-971; Lombardino, J. G., J. Org. Chem., 1971, 36, 1843-1845; Hlasta, D. J., et al., Tet. Lett., 1991, 32, 49, 7179-7182; Haworth, L., J. Chem. Soc., 1924, 125, 1304; Moulton, J. Am. Chem., 1891, 13, 200; Zincke, G., Justus Liebigs Ann. Chem., 1922, 427,249; Remsen, B., J. Am. Chem., 2, 1880/1881, 411; Schoop, Chem. Ber., 1881, 14, 223; Weber., Chem. Ber., 1892, 25, 1740; Szabo, Bull. Soc. Chim. Fr., 1953, 771-773; Love. K., J. Org. Chem., 1962, 27, 2177-2180; Jocobsen, Justus Liebigs Ann. Chem., 1881, 206, 175; Finzi, C., Gazz. Chim. Ital., 1938, 68, 132-139; Fahlberg, Chem. Ber., 1887, 20, 1603; de Stevens et al., J. Med. Pharm. Chem., 1959, 1, 565-573; Thomae, K., DE 2749640; Hamor, G. H., J. Am. Pharm. Assoc. Sci. Ed. 1960, 49, 280-283; Warren, A. and Hamor, G. H., J. Pharm. Sci., 1961, 50, 625-626; Unterhalt, B. and Moghaddarm, S., Pharmazie, 1994, 49, 2/3, 115-117; Vega. S., et al., Dur. J. Med. Chem. Chim. Ther., 1988, 23, 329-334; Rohm-Hass Co., U.S. Pat. No. 3,562,283; Lewis, S. N., J. HeterocycL Chem., 1971, 8, 591-595; Gilbert, E. E., J. Org. Chem, 1970, 35, 850-852; Shkulev, V. A. et al., J. Pharm. Chem., 1977, 11, 10, 1376-1379; Horii, Patent JP 1683, 1962; Lewis. S. N., J. Heterocycl. Chem., 1971, 8, 591-595; Chekhuta, V. G. et al., J. Org. Chem. USSR, 1967, 3, 1763-1766; Schulze, B. and Muehlstaedt, M., A. Chem., GE, 1988, 28, 10, 362; Alo, B., et al., J. Heterocycl. Chem., 1992, 29, 1, 61-64; Waldner, A., Helv. Chim. Acta. GE, 1989, 72, 1435-1443; Burri, K. F., Helv. Chim. Acta., 1989, 72, 1416-1427; and Zawisza, T. and Malinka, W., Farmaco Ed. Sci., 1986, 41, 9, 676-683.

Alternatively, a compound of Formula (Ia) where E is —C(R⁵)(R⁶)X¹ or —C(R^{5a})(R^{6a})CN, R² is hydrogen, and R¹, R^{1a}, R³, R⁴, R⁵, R^{5a}, R⁶ and R^{6a} are as defined in the Summary of the Invention can be prepared as shown in Scheme 3 below.

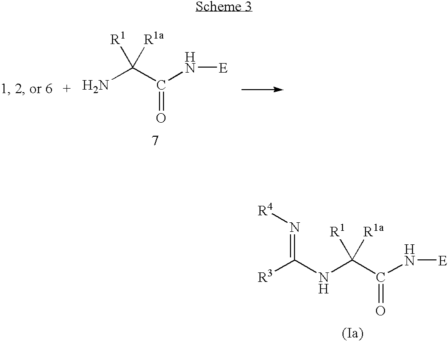

Reaction of a compound of formula 1, 2 or 6 with an amino compound of formula 7 provides a compound of Formula (Ia). The reaction is carried out under the reaction conditions described in Scheme 1 above. Compounds of formula 7 can be prepared by reacting an N-protected amino acid of formula 3 (R′═H) with a compound of formula 5 under the coupling reaction conditions described in Scheme 1 above, followed by removal of the amino protecting group. Suitable amino protecting groups include, but are not limited to, tert-butoxycarbonyl, benzyloxycarbonyl, and the like. Compounds of Formula (Ia) and (Ib) can also be prepared as described in PCT Application Publication Nos. WO 02/20485 and WO 03/029200, and U.S. Pat. No. 6,420,364, the disclosures of which are incorporated herein by reference in their entirety.

Alternatively, a compound of Formula (Ia)/(Ib) where E is C(R⁵)(R⁶)X¹, R² is hydrogen, R³ is hydrogen or a group defined in the Summary of the Invention that contains a basic nitrogen and is bonded to the carbon via the nitrogen atom, and R¹, R^{1a}, R⁴, R⁵, and R⁶ are as defined in the Summary of the Invention can be prepared by proceeding as in the following Reaction Scheme 4 below.

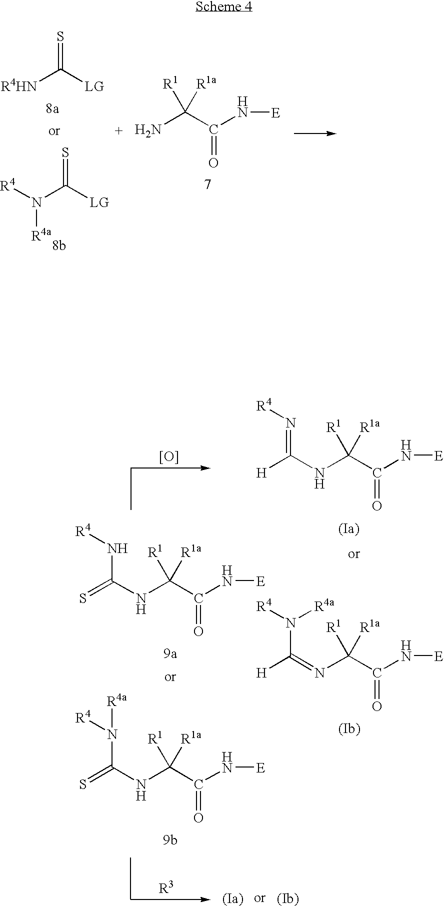

Reaction of a compound of formula 8a or 8b where LG is a suitable leaving group such as imidazol-1-yl with an acid addition salt or free base of a compound of formula 7 provides a compound of formula 9a or 9b respectively. The reaction is carried out in the presence of a base such as diisopropylamine, triethylamine, and the like (if acid addition salt of 7 is used) and in a suitable organic solvent such as methylene chloride, dioxane, and the like. Compounds of formula 8a and 8b can be readily prepared by reacting an amine of formula R⁴NH₂ and R⁴R^{4a}NH respectively, with thio coupling agent such as 1,1′-thiocarbonyldiimidazole, and the like.

Compound 9a or 9b is then converted to a compound of Formula (Ia) or (Ib) by reacting it with a compound of formula R³H where R³ is a group defined in the Summary of the Invention that contains a reactive nitrogen. For example, a compound of Formula (Ia) where R³ is morpholin-1-yl, piperidin-1-yl, or piperazin-1-yl can be prepared by heating a compound formula 9a with morpholine, piperidine, or piperazine respectively, in the presence of copper sulfate on silica gel and a suitable base such as triethylamine, and the like, in a microwave reactor. Suitable reaction solvents include tetrahydrofuran, and the like. Alternatively, 9a or 9b can be reacted with an oxidizing agent such as hydrogen peroxide to give a compound of Formula (Ia) or (Ib) where R³ is hydrogen.

Alternatively, a compound of Formula (Ia) where E is —C(R⁵)(R⁶)X¹, R² is hydrogen, R³ a group defined in the Summary of the Invention that contains a basic nitrogen and is bonded to the carbon via the nitrogen atom, and R¹, R^{1a}, R⁴, R⁵, and R⁶ are as defined in the Summary of the Invention can be prepared by proceeding as in the following Reaction Scheme 5 below.

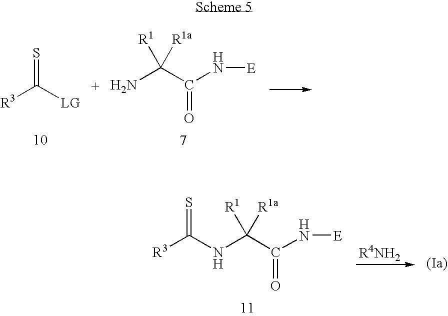

Reaction of a compound of formula 10 where R³ is an amino containing group and is bonded to the carbon via the nitrogen atom with 7 under the presence of a suitable coupling agent such as 2-chloro-1-methylpyridinium iodide provides a compound of formula 11 which is then reacted with an amine of formula R⁴NH₂ where R⁴ is as defined in the Summary of the Invention to provide a compound of Formula (Ia). Compound 10 is prepared as described in Scheme 4 above e.g., reacting morpholine with 1,1′-thiocarbonyldiimidazole.

Compounds of Formula (II) where Q, R^{3c}, R¹, R^{1a}, R², and Z is as defined in the Summary of the Invention and E is —C(R⁵)(R⁶)(R⁷)(R⁸)R¹⁰ where R⁵ and R⁶ are as defined in the Summary of the Invention and R⁷ and R⁸ together form oxo can be prepared by proceeding as illustrated and described in Scheme 6 below.

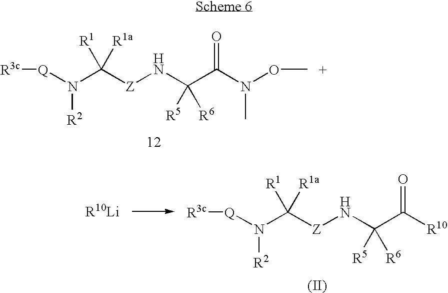

Compounds of Formula (II) where E is —C(R⁵)(R⁶)(R⁷)(R⁸)R¹⁰ where R⁵ and R⁶ are as defined in the Summary of the Invention and R⁷ and R⁸ together form oxo can be prepared by reacting a compound of formula 12 with an organometallic compound of formula R¹⁰Li. The reaction is carried out in a suitable solvent (e.g. tetrahydrofuran (THF), ether, or the like) at −78 to −80° C., preferably at about −78° C., and requires 30 minutes to an hour to complete. The organometallic compound of formula R¹⁰Li is generated by treating a corresponding organo compound or a brominated derivative thereof, with n-butyllithium or tert-butyllithium in a suitable solvent (e.g. THF, ether, or the like) at −78 to −80° C., preferably at about −78° C., for approximately 30 minutes to an hour.

Compounds of formula 12 where Z is —CO— can be prepared by reacting the Weinreb amide derivative of an amino acid of formula 13:

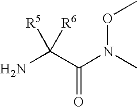

with a compound of the formula R^{3c}QN(R²)C(R¹)(R^{1a})C(O)Y where Q and R^{3c} are as defined in the Summary of the invention and Y is hydroxy or an activating group (succinimide, or the like). When Y is an activating group, the reaction is carried out in the presence of a suitable base (e.g. triethylamine, diisopropylethylamine, or the like) and in a suitable solvent (e.g. acetonitrile, N,N-dimethylformamide (DMF), dichloromethane, or any suitable combination thereof, or the like) at 10 to 30° C., preferably at about 25° C., and requires 24 to 30 hours to complete. When Y is hydrogen a suitable coupling agent (e.g. benzotriazole-1-yloxy-trispyrrolidinophosphonium hexafluorophosphate (PyBOP®), 1-(3-dimethylaminopropyl)-3-ethylcarbodiimide hydrochloride (EDC), O-benzotriazol-1-yl-N,N,N′,N′-tetramethyluronium hexafluorophosphate (HBTU), O-(7-azabenzotriazol-1-yl)-1,1,3,3-tetramethyluronium hexafluorophosphate (HATU), 1,3-dicyclohexylcarbodiimide (DCC), or the like) and a base (e.g. N,N-diisopropylethylamine, triethylamine, or the like) is required and the reaction takes about 2 to 3 hours to complete.

Compounds formula 13 can be prepared by reacting a corresponding N-protected alpha amino acid with N,O-dimethylhydroxylamine hydrochloride followed by deprotection of the amino group. The reaction with the N,O-dimethylhydroxylamine is carried out in the presence of a suitable coupling agent (PyBOP®, EDC, HBTU, DCC, and the like) and a base (e.g. N,N-diisopropylethylamine, triethylamine, or the like) in a suitable solvent (e.g. dichloromethane, DMF, and the like) at 20 to 30° C., preferably at about 25° C., and takes about 2 to 4 hours to complete. Deprotection of the amino group provides the desired compound 13.

Compounds of formula R^{3c}QN(R²)C(R¹)(R^{1a})C(O)Y can be prepared by reacting a carboxy protected amino acid of formula NH₂(R²)C(R¹)(R^{1a})C(O)OPG where PG is a suitable carboxy protecting group with an acylating agent, a sulfonylating agent, a carbamoyl halide, or sulfamoyl halide of formula R^{3c}COL, R^{3c}SO₂L, R^{3c}NHCOL, or R^{3c}NHSO₂L respectively under conditions well known in the art. Removal of the carboxy protecting group provides R^{3c}QN(R²)C(R¹)(R^{1a})C(O)OH which is then reacted with compound 13.

Alternately, compounds of Formula (II) where E is —C(R⁵)(R⁶)C(R⁷)(R⁸)R¹⁰ where R⁵, R⁶, R⁷ and R⁸, and other groups are as defined in the Summary of the Invention can be prepared by proceeding as illustrated and described in Scheme 7 below:

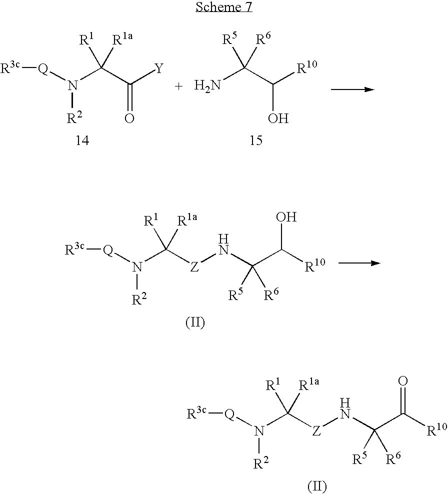

Reaction of a compound of formula 14 where Y is hydroxy or an activating group (e.g. 2,5-dioxopyrrolidin-1-yl, succinimide, or the like) with a compound of formula 15 under the reaction conditions described in Scheme 6 above provides a compound of Formula (II).

Compounds of formula 15 can be prepared under deprotonation reaction conditions by treating benzoxazole, oxazolo[4,5-b]pyridine, 2-pyridin-3-yloxadiazole, 2-pyridin-4-yl-oxadiazole, 2-phenyloxadiazole, and the like, with a Grignard reagent such as isopropylmagnesium chloride and then reacting the resulting organomagnesium reagent with an alpha-(N-protected amino)aldehyde of formula CR⁵R⁶(NHPG)CHO, where PG is a suitable amino protecting group (such as tert-butyoxycarbonyl, benzyloxycarbonyl, or benzyl) to provide an N-protected compound of formula 13 after treatment with an aqueous acid or buffer. Removal of the amino protecting group then provides a compound of formula 15.

The addition reaction is typically carried out in an ethereal organic solvent such as tetrahydrofuran, diethyl ether, dioxane, and the like, preferably tetrahydrofuran, at a temperature from about −78° C. to about 40° C. Preferably, the reaction is carried out from about −10° C. to about 40° C., more preferably from about −10° C. to about 10° C. The reaction typically requires an hour to complete. The nucleophilic addition reaction is typically carried out from about −10° C. to about room temperature. Compounds of formula CR⁵R⁶(NHPG)CHO are prepared from commercially available starting materials by methods well known in the art.

The reaction conditions employed for removal of the amino protecting group depends on the nature of the protecting group. For example, if the protecting group is tert-butoxycarbonyl, it is removed under acid reaction conditions. Suitable acids are trifluoroacetic acid (TFA), hydrochloric acid, and the like. If the protecting group is benzyl or benzyloxycarbonyl, it is removed under catalytic hydrogenation reaction conditions. Suitable catalyst are palladium, platinum, rodium based catalysts and others known in the art. Other suitable reaction conditions for their removal can be found in Greene, T. W.; and Wuts, P. G. M.; Protecting Groups in Organic Synthesis; John Wiley & Sons, Inc. 1999. The reaction is carried out in an inert organic solvent methylene chloride, tetrahydrofuran, dioxane, dimethylformamide, and the like.

Oxidation of hydroxy group in (II) where R⁷ is hydroxy and R⁸ is hydrogen with a suitable oxidizing agent such as Dess-Martin Periodinane in a halogenated organic solvent such as methylene chloride, chloroform, carbon tetrachloride, and the like, or a mixture of TEMPO/bleach then provides a corresponding compound of Formula (II) where R⁷ and R⁸ together form oxo.

The above procedure can be used to prepare compounds of Formula (II) where E is —C(R^{5a})(R^{6a})CN by substituting compound 15 with NH₂C(R^{5a})(R^{6a})CN.

Other methods for preparing compounds of Formula (II) are described in U.S. Pat. No. 6,576,630 and PCT application publication No. WO 00/55126 the disclosures of which are incorporated herein by reference in their entirety.

Compounds of Formula (III) where R^{3d}, R^{3e}, and E are as defined in the Summary of the Invention can be prepared by proceeding as illustrated and described in Scheme 8 below:

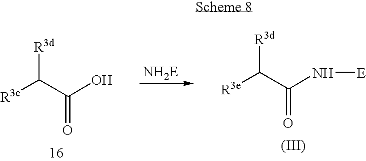

Compounds of Formula (III) can be prepared by reacting an acid of formula 16 with an amino compound of formula NH₂E where E is as defined in the Summary of the Invention under conditions described in Scheme 6 above.

Compounds of formula 16 can be prepared by reacting a compound of formula 17:

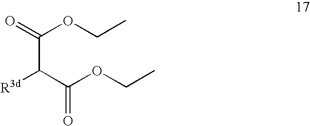

with R^{3e}L where L is a leaving group and R^{3d} and R^{3e} are as defined in the Summary of the Invention. The reaction involves coupling (or alkylation) followed by alkaline hydrolysis at a temperature during which the dicarboxylic acid formed undergoes mono-decarboxylation. The coupling reaction can be carried out in the presence of a suitable base (e.g. triethylamine) in a suitable solvent (e.g. ethanol). The decarbalkoxylation can be affected under strongly basic conditions (e.g. in the presence of 1N aqueous sodium hydroxide) in a suitable solvent (e.g. ethanol). Detailed description for the syntheses of compounds of Formula (III) by this process is described in WO 02/051983.

Compounds of formula 17, in which R^{3d} and R^{3e} are benzylsulfonylmethyl, can be prepared by reacting a compound of formula 18: 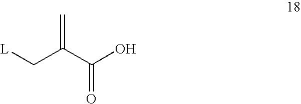
in which L is a halo group, with benzyl mercaptan under strongly basic conditions to produce a compound of formula 19: 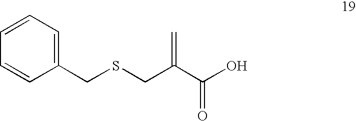
followed by reaction of 19 with benzyl mercaptan in the presence of a suitable coupling reagent (e.g. DMAP) and in a suitable solvent (e.g. DMF). A detailed description of the synthesis of a compound of formula 16 by a similar process as that described above is set forth WO 02/051983.

Compounds of formula 16, in which R^{3d} is biaryl, can be prepared by coupling a compound of formula 18: 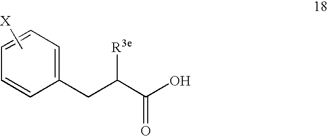
in which X is a halo group and R^{3e} is as defined in the Summary of the Invention, with a compound of ArL, in which Ar is an aryl group and L is a leaving group, to produce a compound of formula 18 in which R^{3d} is biaryl. The coupling reaction takes place in the presence of a suitable catalyst (e.g. tetrakis-triphenylphosphine palladium). A detailed description of the synthesis of a compound of formula 18 by a similar process as that described above is set forth WO 02/051983.

Compounds of Formula (IV) where R^{3g} is —NHR⁴⁸ where R⁴⁸ is other than hydrogen and R^{3f} and E are as defined in the Summary of the Invention can be prepared by proceeding as illustrated and described in Scheme 9 below:

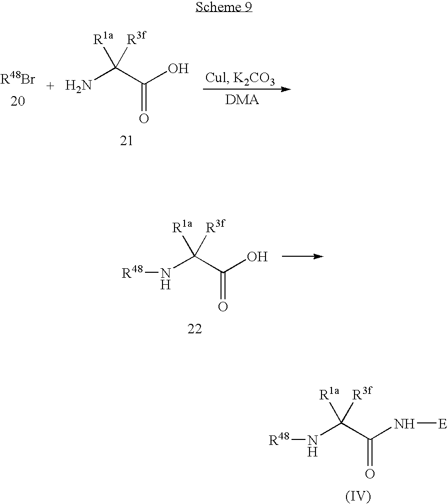

Treatment of a compound of formula 20 with alpha aminoacetic acid of formula 21 in the presence of copper iodide and a base such as potassium carbonate provides a compound of formula 22. The reaction is carried out in a suitable organic solvent such as dimethylacetamide, and the like.

Compounds of formula 20 and 21 are commercially available or they can be prepared by methods well known in the art.

Treatment of 22 with an amino compound of formula NH₂E where E is as defined in the Summary of the Invention provides a compound of Formula (IV). The reaction is carried out in the presence of a coupling agent under the reaction conditions as described above.

A compound of Formula (IV) where R^{3g} is —R⁴⁶ or —NR⁴⁷R⁴⁸ can be prepared as illustrated and described in Scheme 10 below.

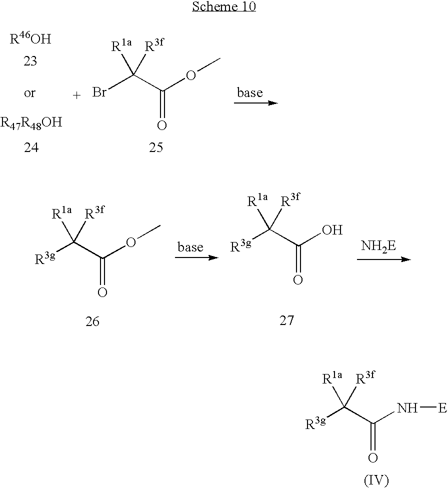

Treatment of a compound of formula 23 or 24 where R⁴⁶, R⁴⁷ and R⁴⁸ are as defined in the Summary of the Invention with 2-bromoacetate of formula 25 provides a compound of formula 26 where R^{3g} is —OR⁴⁶ or —NR⁴⁷R⁴⁸ respectively. The reaction is carried out in the presence of a strong non-nucleophilic base such as sodium hydride, tert-butoxide, and the like and in a sutiable organic solvent such as dimethylformamide, tetrahydrofuran, and the like. Hydrolysis of the ester group in 26 under basic hydrolysis reaction conditions provides a compound of formula 27. Suitable bases are aqueous lithium hydroxide, sodium hydroxide, and the like. Suitable solvents are alcoholic solvents such as methanol, ethanol, and the like. A compound of formula 27 can then be converted to a corresponding compound of Formula (IV) as described above.

Alternate methods for preparing compounds of Formula (IV) are disclosed in WO 02/098850, the disclosure of which is incorporated herein by reference in its entirety. Additional Processes for Preparing Compounds of Formulae (I)-(IV):

A compound of the present invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the present invention can be prepared by reacting the free acid form of the compound with a pharmaceutically acceptable inorganic or organic base. Inorganic and organic acids and bases suitable for the preparation of the pharmaceutically acceptable salts of compounds of the present invention are set forth in the definitions section of this Application. Alternatively, the salt forms of the compounds of the present invention can be prepared using salts of the starting materials or intermediates.

The free acid or free base forms of the compounds of the present invention can be prepared from the corresponding base addition salt or acid addition salt form. For example, a compound of the present invention in an acid addition salt form can be converted to the corresponding free base by treating with a suitable base (e.g., ammonium hydroxide solution, sodium hydroxide, and the like). A compound of the present invention in a base addition salt form can be converted to the corresponding free acid by treating with a suitable acid (e.g., hydrochloric acid, etc).

The N-oxides of the compounds of the present invention can be prepared by methods known to those of ordinary skill in the art. For example, N-oxides can be prepared by treating an unoxidized form of the compound of the present invention with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds of the present invention can be prepared from the N-oxide of an appropriate starting material.

Compounds of the present invention in unoxidized form can be prepared from N-oxides of compounds of the present invention by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds of the present invention can be prepared by methods known to those of ordinary skill in the art (e.g., for further details see Saulnier et al.(1994), Bioorganic and Medicinal Chemistry Letters, Vol. 4, p. 1985). For example, appropriate prodrugs can be prepared by reacting a non-derivatized compound of the present invention with a suitable carbamylating agent (e.g., 1,1-acyloxyalkylcarbonochloridate, para-nitrophenyl carbonate, or the like).

Protected derivatives of the compounds of the present invention can be made by means known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

Compounds of the present invention may be conveniently prepared, or formed during the process of the invention, as solvates (e.g. hydrates). Hydrates of compounds of the present invention may be conveniently prepared by recrystallisation from an aqueous/organic solvent mixture, using organic solvents such as dioxin, tetrahydrofuran or methanol.

Compounds of the present invention can be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diasteromeric derivatives of compounds of the present invention, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

Preparation of Biological Agents

In practicing this invention several processes for the generation or purification of biological agents are used. Methods for preparing the biologics are well known in the art as discussed below.

Monoclonal antibodies are prepared using standard techniques, well known in the art, such as by the method of Kohler and Milstein, Nature 1975, 256:495, or a modification thereof, such as described by Buck et al. 1982, In Vitro 18:377. Typically, a mouse or rat is immunized with the MenB PS derivative conjugated to a protein carrier, boosted and the spleen (and optionally several large lymph nodes) removed and dissociated into single cells. If desired, the spleen cells may be screened (after removal of non-specifically adherent cells) by applying a cell suspension to a plate or well coated with the antigen. B-cells, expressing membrane-bound immunoglobulin specific for the antigen, will bind to the plate, and will not be rinsed away with the rest of the suspension. Resulting B-cells, or all dissociated spleen cells, are then induced to fuse with myeloma cells to form hybridomas. Representative murine myeloma lines for use in the hybridizations include those available from the American Type Culture Collection (ATCC).

Chimeric antibodies composed of human and non-human amino acid sequences may be formed from the mouse monoclonal antibody molecules to reduce their immunogenicity in humans (Winter et al. Nature 1991, 349:293; Lobuglio et al. Proc. Nat. Acad. Sci. USA 1989, 86:4220; Shaw et al. J. Immunol. 1987, 138:4534; and Brown et al. Cancer Res. 1987, 47:3577; Riechmann et al. Nature 1988, 332:323; Verhoeyen et al. Science 1988, 239:1534; and Jones et al. Nature 1986, 321:522; EP Publication No.519,596, published Dec. 23, 1992; and U.K. Patent Publication No. GB 2,276,169, published Sep. 21, 1994).

Antibody molecule fragments, e.g., F(ab′).sub.2, FV, and sFv molecules, that are capable of exhibiting immunological binding properties of the parent monoclonal antibody molecule can be produced using known techniques. Inbar et al. Proc. Nat. Acad. Sci. USA 1972, 69:2659; Hochman et al. Biochem. 1976, 15:2706; Ehrlich et al. Biochem. 1980, 19:4091; Huston et al. Proc. Nat. Acad. Sci. USA 1988, 85(16):5879; and U.S. Pat. Nos. 5,091,513 and 5,132,405, to Huston et al.; and U.S. Pat. No. 4,946,778, to Ladner et al.

In the alternative, a phage-display system can be used to expand the monoclonal antibody molecule populations in vitro. Saiki, et al. Nature 1986, 324:163; Scharf et al. Science 1986, 233:1076; U.S. Pat. Nos. 4,683,195 and 4,683,202; Yang et al. J. Mol. Biol. 1995,254:392; Barbas, III et al. Methods: Comp. Meth Enzymol. 1995, 8:94; Barbas, III et al. Proc. Natl. Acad. Sci. USA 1991, 88:7978.

The coding sequences for the heavy and light chain portions of the Fab molecules selected from the phage display library can be isolated or synthesized, and cloned into any suitable vector or replicon for expression. Any suitable expression system can be used, including, for example, bacterial, yeast, insect, amphibian and mammalian systems. Expression systems in bacteria include those described in Chang et al. Nature 1978, 275:615, Goeddel et al. Nature 1979, 281:544, Goeddel et al. Nucleic Acids Res. 1980, 8:4057, European Application No. EP 36,776, U.S. Pat. No. 4,551,433, deBoer et al. Proc. Natl. Acad. Sci. USA 1983, 80:21-25, and Siebenlist et al. Cell 1980, 20:269.

Expression systems in yeast include those described in Hinnen et al. Proc. Natl. Acad. Sci. USA 1978 75:1929, Ito et al. J. Bacteriol. 1983, 153:163, Kurtz et al. Mol. Cell. Biol. 1986, 6:142, Kunze et al. J. Basic Microbiol. 1985, 25:141, Gleeson et al. J. Gen. Microbiol. 4986, 132:3459, Roggenkamp et al. Mol. Gen. Genet. 1986, 202:302, Das et al. J. Bacteriol. 1984,158:1165, De Louvencourt et al. J. Bacteriol. 1983, 154:737, Van den Berg et al. Bio/Technology 1990, 8:135, Kunze et al. J. Basic Microbiol. 1985, 25:141, Cregg et al. Mol. Cell. Biol. 1985, 5:3376, U.S. Pat. Nos. 4,837,148 and 4,929,555, Beach et al. Nature 1981, 300:706, Davidow et al. Curr. Genet. 1985, 10:380, Gaillaidin et al. Curr. Genet. 1985, 10:49, Ballance et al. Biochem. Biophys. Res. Commun. 1983, 112:284-289, Tilburn et al. Gene 1983, 26:205-221, Yelton et al. Proc. Natl. Acad. Sci. USA 1984, 81:1470-1474, Kelly et al. EMBO J. 1985, 4:475479; European Application No. EP 244,234, and International Publication No. WO 91/00357.

Expression of heterologous genes in insects can be accomplished as described in U.S. Pat. No. 4,745,051, European Application Nos. EP 127,839 and EP 155,476, Vlak et al. J. Gen. Virol. 1988, 69:765-776, Miller et al. Ann. Rev. Microbiol. 1988, 42:177, Carbonell et al. Gene 1988, 73:409, Maeda et al. Nature 1985, 315:592-594, Lebacq-Verheyden et al. Mol. Cell. Biol. 1988, 8:3129, Smith et al. Proc. Natl. Acad. Sci. USA 1985, 82:8404, Miyajima et al. Gene 1987, 58:273, and Martin et al. DNA 1988, 7:99. Numerous baculoviral strains and variants and corresponding permissive insect host cells from hosts are described in Luckow et al. Bio/Technology 1988, 6:47-55, Miller et al. GENERIC ENGINEERING, Setlow, J. K. et al. eds., Vol. 8, Plenum Publishing, pp. 1986, 277-279, and Maeda et al. Nature 1985, 315:592-594.

Mammalian expression can be accomplished as described in Dijkema et al. EMBO J. 1985, 4:761, Gorman et al. Proc. Natl. Acad. Sci. USA 1982, 79:6777, Boshart et al. Cell 1985, 41:521, and U.S. Pat. No. 4,399,216. Other features of mammalian expression can be facilitated as described in Ham et al. Meth. Enz. 1979, 58:44, Barnes et al. Anal. Biochem. 1980, 102:255, U.S. Pat. Nos. 4,767,704, 4,657,866, 4,927,762, 4,560,655 and Reissued U.S. Pat. No. RE 30,985, and in International Publication Nos. WO 90/103430, WO 87/00195.

The production of recombinant adenoviral vectors are described in U.S. Pat. No. 6,485,958. Botulinum toxin type A can be obtained by establishing and growing cultures of Clostridium botulinum in a fermenter and then harvesting and purifying the fermented mixture in accordance with known procedures.

Any of the above-described protein production methods can be used to provide the biologic that would benefit from the present invention.

### Testing

The cysteine protease inhibitory activity, in particular, the Cathepsin S inhibitory activities of the compounds of the invention can be determined by methods known to those of ordinary skill in the art. Suitable in vitro assays for measuring protease activity and the inhibition thereof by test compounds are known. Typically, the assay measures protease-induced hydrolysis of a peptide-based substrate. Details of assays for measuring protease inhibitory activity are set forth in Biological Examples 1-5, infra.

### Administration and Pharmaceutical Compositions

In general, a compound of the present invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. For example, therapeutically effective amounts of a compound of compounds of the present invention may range from about 10 micrograms per kilogram body weight (μg/kg) per day to about 20 milligram per kilogram body weight (mg/kg) per day, typically from about 100 μg/kg/day to about 10 mg/kg/day. Therefore, a therapeutically effective amount for a 80 kg human patient may range from about 1 mg/day to about 1.6 g/day, typically from about 1 mg/day to about 100 mg/day. In general, one of ordinary skill in the art, acting in reliance upon personal knowledge and the disclosure of this Application, will be able to ascertain a therapeutically effective amount of a compound of the present invention for treating a given disease.

The compounds of the present invention can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of the present invention in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, and the like). Preferred liquid carriers, particularly for injectable solutions, include water, saline, aqueous dextrose and glycols.

The amount of a compound of the present invention in the composition may vary widely depending upon the type of formulation, size of a unit dosage, kind of excipients and other factors known to those of skill in the art of pharmaceutical sciences. In general, a composition of a compound of the present invention for treating a given disease will comprise from 0.01% w to 10% w, preferably 0.3% w to 1% w, of active ingredient with the remainder being the excipient or excipients. Preferably the pharmaceutical composition is administered in a single unit dosage form for continuous treatment or in a single unit dosage form ad libitum when relief of symptoms is specifically required. Representative pharmaceutical formulations containing a compound of the present invention are described in Example 1 below.

As stated previously, the compounds of this invention can be administered in combination with biologics that are selected for their particular usefulness against the condition that is being treated.

### EXAMPLES

### Biological Examples

### Example 1

### Cathepsin B Assay

Solutions of test compounds in varying concentrations were prepared in 10 μL of dimethyl sulfoxide (DMSO) and then diluted into assay buffer (40 μL, comprising: N,N-bis(2-hydroxyethyl)-2-aminoethanesulfonic acid (BES), 50 mM (pH 6); polyoxyethylenesorbitan monolaurate, 0.05%; and dithiothreitol (DTT), 2.5 mM). Human cathepsin B (0.025 pMoles in 25 μL of assay buffer) was added to the dilutions. The assay solutions were mixed for 5-10 seconds on a shaker plate, covered and incubated for 30 minutes at room temperature. Z-FR-AMC (20 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

Compounds of the invention were tested by the above-described assay and observed to exhibit cathepsin B inhibitory activity.

### Example 2

### Cathepsin K Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin K (0.0906 pMoles in 25 μL of assay buffer) was added to the dilutions. The assay solutions were mixed for 5-10 seconds on a shaker plate, covered and incubated for 30 minutes at room temperature. Z-Phe-Arg-AMC (4 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

Compounds of the invention were tested by the above-described assay and observed to exhibit cathepsin K inhibitory activity.

### Example 3

### Cathepsin L Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin L (0.05 pMoles in 25 μL of assay buffer) was added to the dilutions. The assay solutions were mixed for 5-10 seconds on a shaker plate, covered and incubated for 30 minutes at room temperature. Z-Phe-Arg-AMC (1 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

Compounds of the invention were tested by the above-described assay and observed to exhibit cathepsin L inhibitory activity.

### Example 4

### Cathepsin S Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); β-mercaptoethanol, 2.5 mM; and BSA, 0.00%. Human cathepsin S (0.05 pMoles in 25 μL of assay buffer) was added to the dilutions. The assay solutions were mixed for 5-10 seconds on a shaker plate, covered and incubated for 30 minutes at room temperature. Z-Val-Val-Arg-AMC (4 nMoles in 25 μL of assay buffer containing 10% DMSO) was added to the assay solutions and hydrolysis was followed spectrophotometrically (at λ460 nm) for 5 minutes. Apparent inhibition constants (Kᵢ) were calculated from the enzyme progress curves using standard mathematical models.

Compounds of the invention were tested by the above-described assay and observed to exhibit cathepsin S inhibitory activity.

### Example 5

### Cathepsin F Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); DTT, 2.5 mM; and BSA, 0.01%. Human cathepsin F (0.1 pMoles in 25 μL of assay buffer) was added to the dilutions. The assay solutions were mixed for 5-10 seconds on a shaker plate, covered and incubated for 30 minutes at room temperature. Z-Phe-Arg-AMC (2 nMoles in 25 μL of assay buffer containing 10% DMSO) was added to the assay solutions and hydrolysis was followed spectrophotometrically (at λ460 mm) for 5 minutes. Apparent inhibition constants (Kᵢ) were calculated from the enzyme progress curves using standard mathematical models.

Compounds of the invention were tested by the above-described assay and observed to exhibit cathepsin F inhibitory activity.

### Example 6

### In Vitro Lip10 Accumulation Assay

During normal antigen presentation, Lip10 is proteolytically degraded to enable loading of a peptide fragment and subsequent MHC-II presentation on the surface of antigen presenting cells. The cleavage process is mediated by Cathepsin S. Thus, the lip10 assay is an in vitro measure of a compound's ability to block cathepsin S and by extension antigen presentation. A compound that causes the accumulation of Lip10 at low concentration would be expected to block presentation of antigens.

Method:

Raji cells (4×10⁶) were cultured with 0.02% DMSO or different concentrations of Cathepsin S inhibitors in RPMI medium 1640 containing 10% (v/v) FBS, 10 mM HEPES, 2 mM L-glutamine, and 1 mM sodium pyruvate for four hours at 37° C. in 5% CO₂ humidified atmosphere. After the culture period, cells were washed with cold PBS and cells were then lysed in NP-40 lysis buffer (5 mM EDTA, 1% NP-40, 150 mM NaCl, and 50 mM Tris, pH 7.6) with protease inhibitors. Protein determinations were performed and lysate samples were boiled in reducing SDS sample buffer. Proteins were separated by electrophoresis on 12% NuPAGE® Bis-Tris gels. Proteins were then transferred to nitrocellulose membranes, and after incubation with blocking buffer (5% non-fat dry milk in PBS-Tween), the blots were incubated with the primary antibody against human CD74 invariant chain synthetic peptide (1.5 to 2 μg/ml of mouse anti-CD74 monoclonal antibody, PIN.1, Stressgen Biotechnologies). Blots were then incubated with the secondary antibody, horseradish peroxidase conjugated donkey anti-mouse IgG, at a 1:1 0,000 dilution. Immunoreactive proteins were detected by chemiluminescense reaction using Pierce Super Signal® West Pico chemiluminescense substrate.

### Pharmaceutical Composition Examples

The following are representative pharmaceutical formulations containing a compound of the present invention.

### Tablet Formulation

The following ingredients are mixed intimately and pressed into single scored tablets.

| | Quantity per |
|---|---|
| Ingredient | tablet, mg |
| compound of this invention | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

### Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution) | 12.85 | g |
| Veegum K (Vanderbilt Co.) | 1.0 | g |
| flavoring | 0.035 | mL |
| colorings | 0.5 | mg |
| distilled water | q.s. to 100 | mL |

### Injectable Formulation

The following ingredients are mixed to form an injectable formulation.

| Ingredient | Amount |
|---|---|
| compound of this invention | 1.2 g |
| sodium acetate buffer solution, | 0.4 M 2.0 mL |
| HCl (1 N) or NaOH (1 N) | q.s. to suitable pH |
| water (distilled, sterile) | q.s. to 20 mL |

All of the above ingredients, except water, are combined and heated to 60-70° C. with stirring. A sufficient quantity of water at 60° C. is then added with vigorous stirring to emulsify the ingredients, and water then added q.s. to 100 g.

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| | |
|---|---|
| compound of the invention | 500 mg |
| Witepsol ® H-15 | balance |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications and publications cited in this application including U.S. Provisional Application Ser. Nos. 60/528,846 and 60/532,202 filed on Dec. 11, 2003 and Dec. 23, 2003 respectively are hereby incorporated by reference in their entirety for all purposes to the same extent as if each individual patent, patent application or publication were so individually denoted.

What is claimed:

1. A method of treating a patient undergoing a non-tissue graft therapy wherein the therapy may or does induce a deleterious immune response in the patient which method comprises administering to the patient a Cathepsin S inhibitor.

2. A method of treating a patient undergoing a non-tissue graft therapy wherein the therapy induces a deleterious immune response in the patient comprising administering to the patient a Cathepsin S inhibitor.

3. The method of claim 1 or 2 wherein the therapy involves treatment of the patient with a small molecule therapeutic.

4. The method of claim 3 wherein the small molecule therapeutic is heparin, low molecular weight heparin, procainamide, or hydralazine.

5. The method of claim 1 or 2 wherein the therapy involves treatment of the patient with a biologic.

6. The method of claim 5 wherein the biologic is a protein.

7. The method of claim 5 wherein the biologic is an antibody.

8. The method of claim 5 wherein the biologic is Remicade®, Refacto®, Referon-A®, Factor VIII, Factor VII, Betaseron®, Epogen®, Embrel®, Interferon beta, Botox®, Fabrazyme®, Elspar®, Cerezyme®, Myobloc®, Aldurazyme®, Verluma®, Interferon alpha, Humira®, Aranesp®, Zevalin® or OKT3.

9. A method of treating immune response in a patient caused by administration of a small molecule therapeutic or a biologic to the patient which method comprises administering to the patient in need of such treatment a therapeutically effective amount of a Cathepsin S inhibitor.

10. A method of treating a patient undergoing treatment with a biologic with a Cathepsin S inhibitor.

11. The method of claim 9 wherein the immune response is caused by a small molecule therapeutic.

12. The method of claim 11 wherein the small molecule therapeutic is heparin, low molecular weight heparin, procainamide, or hydralazine.

13. The method of claim 9 wherein the immune response is caused by a biologic.

14. The method of claim 10 or 13 wherein the biologic is a protein.

15. The method of claim 14 wherein the biologic is an antibody.

16. The method of claim 14 wherein the biologic is Remicade®, Refacto®, Referon-A®, Factor VIII, Factor VII, Betaseron®, Epogen®, Embrel®, Interferon beta, Botox®, Fabrazyme®, Elspar®, Cerezyme®, Myobloc®, Aldurazyme®, Verluma®, Interferon alpha, Humira®, Aranesp®, Zevalin® or OKT3.

17. The method of any of the claims 1-8 wherein the Cathepsin S inhibitor is administered prior to, concomitantly or after the therapy.

18. The method of any of the claims 9, 11, or 12 wherein the Cathepsin S inhibitor is administered prior to, concomitantly, or after the administration of the small molecule therapeutic.

19. The method of any of the claims 9, 10, and 13-16 wherein the Cathepsin S inhibitor is administered prior to, concomitantly, or after the administration of the biologic.

20. The method of any of the claims 1-19 wherein the Cathepsin S inhibitor is:
(a) a compound of Formula (Ia) or (Ib): 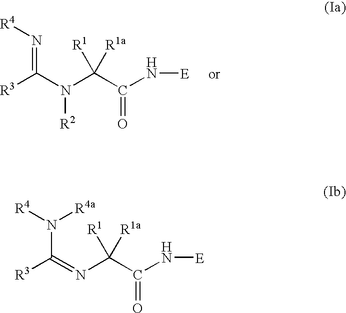
wherein:
E is:
(i) —C(R⁵)(R⁶)X¹ where X¹ is —CHO, —C(R⁷)(R⁸)CF₃, —C(R⁷)(R⁸)CF₂CF₂R⁹, —C(R⁷)(R⁸)R¹⁰, —C(O)C(O)R¹⁰, —CH═CHS(O)₂R¹⁰, —C(R⁷)(R⁸)C(R⁷)(R⁸)OR¹⁰, —C(R⁷)(R⁸)CH₂OR¹⁰, —C(R⁷)(R⁸)C(R⁷)(R⁸)R¹⁰, —C(R⁷)(R⁸)CH₂N(R¹¹)SO₂R¹⁰, —C(R⁷)(R⁸)CF₂C(O)NR¹⁰R¹¹, —C(R⁷)(R⁸)C(O)NR¹⁰R¹¹, —C(R⁷)(R⁸)C(O)N(R¹¹)(CH₂)₂OR¹¹, or —C(R⁷)(R⁸)C(O)N(R¹¹)(CH₂)₂NR¹⁰R¹¹, or
(ii) —C(R^{5a})(R^{6a})CN;
where:
R⁵ and R^{5a} are independently hydrogen or alkyl;
R⁶ and R^{6a} are independently selected from the group consisting of hydrogen, alkyl, haloalkyl, cycloalkyl, cycloalkylalkyl, aryl, aralkyl, heteroaryl, heteroaralkyl, heterocycloalkyl, heterocycloalkylalkyl, -alkylene-X—R¹² (where X is —O—, —NR¹³—, —S(O)ₙ₁—, —CONR¹³—, —NR¹³CO—, —NR¹³C(O)O—, —NR¹³CONR¹³—, —OCONR¹³—, —NR¹³SO₂—, —SO₂NR¹³—, —NR¹³SO₂NR¹³—, —CO—, —OCO—, or —C(O)O— where n1 is 0-2, R¹² hydrogen, alkyl, haloalkyl, cycloalkyl, cycloalkylalkyl, heterocycloalkyl, heterocycloalkylalkyl, aryl, aralkyl, heteroaryl, or heteroaralkyl and each R¹³ is hydrogen or alkyl) wherein the aromatic or alicyclic ring in R⁶ and R^{6a} is optionally substituted with one, two, or three R^{a} independently selected from alkyl, haloalkyl, alkoxy, hydroxy, haloalkoxy, halo, carboxy, alkoxycarbonyl, amino, monsubstituted amino, disubstituted amino, nitro, aryloxy, benzyloxy, acyl, alkylsulfonyl, or arylsulfonyl where the aromatic or alicyclic ring in R^{a} is optionally substituted with one or two substituents independently selected from alkyl, halo, alkoxy, haloalkyl, haloalkoxy, hydroxy, amino, alkylamino, dialkylamino, carboxy, or alkoxycarbonyl; or
R⁵ and R⁶ and R^{5a} and R^{6a} taken together with the carbon atom to which both R⁵ and R⁶ and R^{5a} and R^{6a} are attached form (i) cycloalkylene optionally substituted with one or two R^{b} independently selected from alkyl, halo, alkylamino, dialkylamino, aryl, aralkyl, cycloalkyl, cycloalkylalkyl, heteroaryl, heteroaralkyl, alkoxycarbonyl, or aryloxycarbonyl or (ii) heterocycloalkylene optionally substituted with one to four alkyl or one or two R^{c} independently selected from alkyl, haloalkyl, hydroxy, hydroxyalkyl, alkoxyalkyl, alkoxyalkyloxyalkyl, aryloxyalkyl, heteroaryloxyalkyl, aminoalkyl, acyl, aryl, aralkyl, heteroaryl, heteroaralkyl, heterocycloalkyl, heterocycloalkylalkyl, cycloalkyl, cycloalkylalkyl, —S(O)ₙ₂R¹⁴-alkylene-S(O)ₙ₂—R¹⁵, —COOR¹⁶, -alkylene-COOR¹⁷, —CONR¹⁸R¹⁹, or -alkylene-CONR²⁰R²¹ (where n2 is 0-2 and R¹⁴—R¹⁷, R¹⁸ and R²⁰ are independently hydrogen, alkyl, haloalkyl, aryl, aralkyl, heteroaryl, heteroaralkyl, cycloalkyl, cycloalkylalkyl, or heterocycloalkyl and R¹⁹ and R²¹ are independently hydrogen or alkyl) wherein the aromatic or alicyclic ring in the groups attached to cycloalkylene or heterocycloalkylene is optionally substituted with one, two, or three substituents independently selected from alkyl, haloalkyl, cycloalkyl, cycloalkylalkyl, benzyl, alkoxy, hydroxy, haloalkoxy, halo, carboxy, alkoxycarbonyl, amino, monsubstituted amino, disubstituted amino, or acyl;
R⁷ is hydrogen or alkyl;
R⁸ is hydroxy; or
R⁷ and R⁸ together form oxo;
R⁹ is hydrogen, halo, alkyl, aralkyl or heteroaralkyl;
R¹⁰ is alkyl, aryl, aralkyl, heteroaryl, heteroaralkyl, cycloalkyl, cycloalkylalkyl, heterocycloalkyl, or heterocycloalkylalkyl wherein the aromatic or alicyclic ring in R¹⁰ is optionally substituted with one, two, or three R^{d} independently selected from alkyl, haloalkyl, alkoxy, alkoxyalkyl, cycloalkyl, hydroxy, haloalkoxy, halo, carboxy, alkoxycarbonyl, aminosulfonyl, alkylsulfonyl, arylsulfonyl, heteroarylsulfonyl, aryl, aralkyl, heteroaryl, amino, monsubstituted amino, disubstituted amino, carbamoyl, or acyl wherein the aromatic or alicyclic ring in R^{d} is optionally substituted with one, two, or three substitutents independently selected from alkyl, haloalkyl, alkoxy, haloalkoxy, halo, hydroxy, carboxy, alkoxycarbonyl, amino, alkylamino, or dialkylamino; and
R¹¹ is hydrogen or alkyl; or
(iii) a group of formula (a): 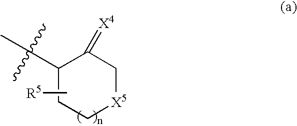
where:
n is 0, 1, or 2;
X⁴ is selected from —NR²²—, —S—, or —O— where R²² is hydrogen, alkyl, or alkoxy; and
X⁵ is —O—, —S—, —SO₂—, or —NR²³— where R²³ is selected from hydrogen, alkyl, haloalkyl, hydroxyalkyl, alkoxyalkyl, aryloxyalkyl, heteroaryloxyalkyl, aminoalkyl, acyl, aryl, aralkyl, heteroaryl, heteroaralkyl, cycloalkyl, cycloalkylalkyl, —S(O)₂R²⁴, -alkylene-S(O)ₙ₃—R²⁵, —COOR²⁶, -alkylene-COOR²⁷, —CONR²⁸R²⁹, or -alkylene-CONR³⁰R³¹ (where n3 is 0-2 and R²⁴—R²⁷, R²⁸ and R³⁰ are independently hydrogen, alkyl, haloalkyl, aryl, aralkyl, heteroaryl, heteroaralkyl, cycloalkyl, cycloalkylalkyl, heterocycloalkyl, or heterocycloalkylalkyl and R²⁹ and R³¹ are independently hydrogen or alkyl) where the aromatic or alicyclic ring in X⁵ is optionally substituted with one, two, or three substituents independently selected from alkyl, haloalkyl, alkoxy, haloalkoxy, halo, hydroxy, amino, alkylamino, dialkylamino, carboxy, or alkoxycarbonyl and one substitutent selected from aryl, aralkyl, heteroaryl, or heteroaralkyl;
R⁵ is as defined above;
R¹ is hydrogen or alkyl;
R^{1a} is hydrogen, alkyl, haloalkyl, cycloalkyl, cycloalkylalkyl, aryl, aralkyl, heteroaryl, heteroaralkyl, heterocycloalkylalkyl, or -alkylene-X²—R³² [wherein X² is —NR³³—, —O—, —S(O)ₙ₄—, —CO—, —COO—, —OCO—, —NR³³CO—, —CONR³³—, —NR³³SO₂—, —SO₂NR³³—, —NR³³COO—, —OCONR³³—, —NR³³CONR³⁴, or —NR³³SO₂NR³⁴— (where R³³ and R³⁴ are independently hydrogen, alkyl, or acyl and n4 is 0-2) and R³² is hydrogen, alkyl, haloalkyl, cycloalkyl, cycloalkylalkyl, heterocycloalkyl, aryl, aralkyl, heteroaryl, heteroaralkyl, heterocycloalkyl, or heterocycloalkylalkyl] wherein said alkylene chain is optionally substituted with one to six halo and wherein the aromatic or alicyclic ring in R^{1a} is optionally substituted with one, two, or three R^{e} independently selected from alkyl, haloalkyl, alkoxy, alkylthio, alkylsulfonyl, arylsulfonyl, aminocarbonyl, aminosulfonyl, acyl, hydroxy, haloalkoxy, halo, nitro, cyano, carboxy, alkoxycarbonyl, aryloxycarbonyl, aryl, heteroaryl, cycloalkyl, cycloalkylalkyl, aralkyl, heteroaralkyl, amino, monsubstituted amino, disubstituted amino, or acyl; or
R¹ and R^{1a} together with the carbon atoms to which they are attached form cycloalkylene or heterocycloalkylene ring wherein said cycloalkylene or heterocycloalkylene is optionally substituted with one or two R^{f} independently selected from alkyl, halo, haloalkyl, hydroxyalkyl, keto, or —SO₂R where R is alkyl, cycloalkyl, cycloalkylalkyl, aryl, aralkyl, heteroaryl or heteroaralkyl where the aromatic or alicylic ring in R^{f} is optionally substituted with one, two, or three substitutents independently selected from alkyl, alkoxy, haloalkyl, haloalkoxy, hydroxy, halo, carboxy, or alkoxycarbonyl;
R² is hydrogen or alkyl;
R³ is hydrogen, alkyl, haloalkyl, cycloalkyl, aryl, aralkyl, heteroaryl, heteroaralkyl, heterocycloalkyl, heterocycloalkylalkyl, amino, mono or disubstituted amino, or -alkylene-X³—R³⁵ [wherein X³ is —NR³⁶—, —O—, —S(O)ₙ₅—, —CO—, —COO—, —OCO—, —NR³⁶CO—, —CONR³⁶—, —NR³⁶SO₂—, —SO₂NR³⁶—, —NR³⁶COO—, —OCONR³⁶—, —NR³⁶CONR³⁷—, or —NR³⁶SO₂NR³⁷— (where R³⁶ and R³⁷ are independently hydrogen, alkyl, or acyl and n5 is 0-2) and R³⁵ is hydrogen, alkyl, haloalkyl, cycloalkyl, cycloalkylalkyl, aryl, aralkyl, heteroaryl, or heteroaralkyl] wherein the aromatic or alicyclic rings in R³ are optionally substituted by one, two, or three R^{g} independently selected from alkyl, halo, hydroxy, alkoxy, haloalkyl, haloalkoxy, oxo, cyano, nitro, acyl, acyloxy, aryl, heteroaryl, cycloalkyl, heterocycloalkyl, aryloxy, benzyloxy, carboxy, alkoxycarbonyl, aryloxycarbonyl, carbamoyl, alkylthio, alkylsulfinyl, alkylsulfonyl, arylthio, arylsulfonyl, arylsulfinyl, alkoxycarbonylamino, aryloxycarbonylamino, alkylcarbamoyloxy, arylcarbamoyloxy, alkylsulfonylamino, arylsulfonylamino, aminosulfonyl, alkylaminosulfonyl, dialkylaminosulfonyl, arylaminosulfonyl, amino, monosubsituted or disubstituted amino, and further wherein the aromatic and alicyclic rings in R^{g} are optionally substituted with one, two, or three R^{h} wherein R^{h} is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, alkylamino, dialkylamino, aryl, heteroaryl, cycloalkyl, carboxy, carboxamido, or alkoxycarbonyl;
R⁴ is hydrogen, alkyl, hydroxy, nitrile, or -(alkylene)ₙ₆-X⁶—R³⁸ (where X⁶ is —O—, —NR³⁹—, —S(O)ₙ₇—, —NR³⁹CO—, —CO—, or —OC(O)— where n6 is 0 or 1, n7 is 0-2, and R³⁹ is hydrogen or alkyl) and R³⁸ is hydrogen, alkyl, phenyl, naphthyl, pyrrolidinyl, piperidinyl, morpholinyl, thiomorpholinyl, piperazinyl, indolinyl, pyranyl, thiopyranyl, furanyl, thienyl, pyrrolyl, oxazolyl, isoxazolyl, thiazolyl, imidazolyl, pyridinyl, pyrimidinyl, pyrazinyl, indolyl, benzofuranyl, benzothienyl, benzimidazolyl, benzthiazolyl, quinolinyl, isoquinolinyl, quinazolinyl, benzoxazolyl, or quinoxalinyl where R³⁸ is optionally substituted with one, two, or three Rⁱ independently selected from alkyl, alkoxy, halo, haloalkyl, haloalkoxy, hydroxy, alkylthio, alkylsulfonyl, arylsulfonyl, aminosulfonyl, acyl, amino, monosubstituted amino, disubstituted amino, carboxy, alkoxycarbonyl, hydroxyalkyl, alkoxyalkyl, aminoalkyl, aryl, heteroaryl, or heterocycloalkyl where the aromatic or alicyclic ring in Rⁱ is optionally substituted with one or two substituents independently selected from alkyl, halo, alkoxy, haloalkyl, haloalkoxy, hydroxy, amino, alkylamino, dialkylamino, carboxy, or alkoxycarbonyl; or
R³ and R⁴ in (Ia) or (Ib) together with the atoms to which they are attached form heteroaryl or heterocycloalkyl ring optionally fused to an aryl or heteroaryl ring wherein said rings are optionally substituted on the aromatic and/or non-aromatic portion of the rings with one, two, or three R^{j};
each R^{j} and R^{4a} is independently:
hydrogen, alkyl optionally interrupted by one or two N, O, C(O), S, S(O), or S(O)₂ and optionally substituted by amino, hydroxy, halo, alkyl, pyrrolidinyl, piperidinyl, morpholinyl, thiomorpholinyl, piperazinyl, indolinyl, pyranyl, thiopyranyl, furanyl, thienyl, pyrrolyl, oxazolyl, isoxazolyl, thiazolyl, imidazolyl, pyridinyl, pyrimidinyl, pyrazinyl, indolyl, benzofuranyl, benzothienyl, benzimidazolyl, benzthiazolyl, quinolinyl, isoquinolinyl, quinazolinyl, benzoxazolyl or quinoxalinyl;
halo, alkoxy, alkylthio, hydroxy, carboxy, aryl, aryloxy, aroyl, heteroaryl, alkanoyl, —C(O)OR where (R is hydrogen, alkyl, alkoxyalkyl, haloalkyl, cycloalkyl, cycloalkylalkyl, heteroaryl, heteroarylalkyl, aryl, arylalkyl, aminoalkyl, heterocycloalkyl, or heterocycloalkylalkyl), aminocarbonyl, aminosulfonyl, alkylsulfonyl, aryloxycarbonyl, benzyloxycarbonyl, alkanoylamino, alkylaminocarbonyl, dialkylaminocarbonyl, alkoxycarbonylamino, aroylamino, amino, alkylamino, dialkylamino, alkylthio, arylthio, alkylsulfonylamino, arylsulfonylamino, alkylaminosulfonyl, arylaminosulfonyl, cycloalkyl, benzyloxy, or ureido wherein each of the aforementioned groups in R^{4a} and R^{j} is optionally substituted with one, two, or three substituents independently selected from halo, hydroxy, alkyl, alkoxy, haloalkyl, haloalkoxy, oxo, carboxy, nitrile, nitro or NH₂C(O)—; or
(b) a compound of Formula (II): 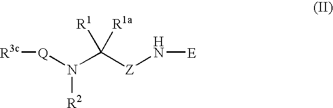
where:
E, R¹, R^{1a} and R² are as defined above;
Z is —CO— or —CH₂SO₂—; or
Q is —CO—, —SO₂—, —OCO—, —NRCO—, or —NRSO₂— where R is hydrogen, alkyl, hydroxyalkyl, alkoxyalkyl, or aralkyl;
R^{3c} is alkyl, haloalkyl, cycloalkyl, cycloalkylalkyl, aryl, aralkyl, heteroaryl, heteroaralkyl, heterocycloalkyl, heterocycloalkylalkyl, or -alkylene-X⁸—R⁴⁰ [wherein X⁸ is —NR⁴¹—, —O—, —S(O)ₙ₈—, —CO—, —COO—, —OCO—, —NR⁴¹CO—, —CONR⁴¹—, —NR⁴¹SO₂—, —SO₂NR⁴¹—, —NR⁴¹COO—, —OCONR⁴¹—, —NR⁴¹CONR⁴²—, or —NR⁴¹SO₂NR⁴²— (where each R⁴¹ and R⁴² is independently hydrogen, alkyl, or acyl and n8 is 0-2) and R⁴⁰ is hydrogen, alkyl, haloalkyl, cycloalkyl, cycloalkylalkyl, heterocycloalkyl, heterocycloalkylalkyl, aryl, aralkyl, heteroaryl, or heteroaralkyl] wherein the alkylene chain in R^{3c} is optionally substituted with one to three halo atoms and the aromatic and alicyclic rings in R^{3c} are optionally substituted by one, two, or three R^{k} independently selected from alkyl, aminoalkyl, halo, hydroxy, alkoxy, haloalkyl, haloalkoxy, oxo, cyano, nitro, acyl, acyloxy, aryl, aralkyl, heteroaryl, heteroaralkyl, cycloalkyl, cycloalkylalkyl, heterocycloalkyl, heterocycloalkylalkyl, aryloxy, benzyloxy, carboxy, alkoxycarbonyl, aryloxycarbonyl, carbamoyl, alkylthio, alkylsulfinyl, alkylsulfonyl, arylthio, arylsulfonyl, arylsulfinyl, alkoxycarbonylamino, aryloxycarbonylamino, alkylcarbamoyloxy, arylcarbamoyloxy, alkylsulfonylamino, arylsulfonylamino, aminosulfonyl, alkylaminosulfonyl, dialkylaminosulfonyl, arylaminosulfonyl, aralkylaminosulfonyl, aminocarbonyl, arylaminocarbonyl, aralkylaminocarbonyl, amino, monosubsituted or disubstituted amino, and further wherein the aromatic and alicyclic rings in R^{k} are optionally substituted with one, two, or three R¹ wherein R¹ is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, monosubstituted amino, dialkylamino, aryl, heteroaryl, cycloalkyl, carboxy, carboxamido, or alkoxycarbonyl; or
(c) a compound of Formula (III): 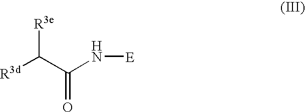
where E is as defined above;
R^{3d} and R^{3e} are independently -alkylene-X⁹—R⁴³ [wherein X⁹ is bond, —NR⁴⁴—, —O—, —S(O)ₙ₉—, —CO—, —COO—, —OCO—, —NR⁴⁴CO—, —CONR⁴⁴—, —NR⁴⁴SO₂—, —SO₂NR⁴⁴—, —NR⁴⁴COO—, —OCONR⁴⁴—, —NR⁴⁴CONR⁴⁵—, or —NR⁴⁴SO₂NR⁴⁵— (where R⁴⁴ and R⁴⁵ are independently hydrogen, alkyl, or acyl and n9 is 0-2) and R⁴³ is hydrogen, alkyl, haloalkyl, cycloalkyl, cycloalkylalkyl, aryl, aralkyl, heteroaryl, heteroaralkyl, heterocycloalkyl, or heterocycloalkylalkyl] wherein the alkylene chain is optionally substituted with one to three halo atoms and the aromatic or alicyclic rings in R^{3d} and R^{3e} are optionally substituted by one, two, or three R^{m} independently selected from alkyl, halo, hydroxy, alkoxy, haloalkyl, haloalkoxy, oxo, cyano, nitro, acyl, acyloxy, carboxy, alkoxycarbonyl, carbamoyl, alkylthio, alkylsulfinyl, alkylsulfonyl, alkoxycarbonylamino, alkylcarbamoyloxy, alkylsulfonylamino, aminosulfonyl, alkylaminosulfonyl, dialkylaminosulfonyl, aminocarbonyl, amino, monosubsituted or disubstituted amino and one R^{m} selected from aryl, aralkyl, heteroaryl, heteroaralkyl, cycloalkyl, cycloalkylalkyl, heterocycloalkyl, heterocycloalkylalkyl, aryloxy, benzyloxy, aryloxycarbonyl, arylthio, arylsulfonyl, arylsulfinyl, aryloxycarbonylamino, arylcarbamoyloxy, arylsulfonylamino, arylaminosulfonyl, or aralkylaminosulfonyl wherein the aromatic or alicyclic ring in R^{m} is optionally substituted with one, two, or three Rⁿ wherein Rⁿ is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, alkylsulfonylamino, arylsulfonylamino, heteroarylsulfonylamino, heteroaralkylsulfonylamino, amino, monosubstituted amino, dialkylamino, aryl, heteroaryl, cycloalkyl, carboxy, carboxamido, or alkoxycarbonyl; or
(d) a compound of Formula (IV): 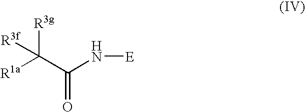
where:
E and R^{1a} are as defined above;
R^{3f} is hydrogen;
R^{3g} is hydrogen, fluoro, —OR⁴⁶ or —NR⁴⁷R⁴⁸ where:
R⁴⁶ is hydrogen, alkyl, cycloalkyl, cycloalkylalkyl, aryl, aralkyl, heteroaryl, heteroaralkyl, heterocycloalkyl, heterocycloalkylalkyl, -(alkylene)n₁₀-X¹⁰—R⁴⁹ [wherein n10 is 0 or 1, X¹⁰ is —CO— or —CONR⁵⁰— where R⁵⁰ is hydrogen, alkyl, or alkoxyalkyl, and R⁴⁹ is hydrogen, alkyl, haloalkyl, alkoxyalkyl, hydroxyalkyl, aryl, aralkyl, cycloalkyl, cycloalkylalkyl, heteroaryl, heteroaralkyl, heterocycloalkyl or heterocycloalkylalkyl or R⁴⁹ and R⁵⁰ together with the nitrogen atom to which they are attached from heterocycloalkyl], or -alkylene-X¹¹—R⁵¹ [wherein X¹¹ is —NR⁵²—, —O—, —S(O)ₙ₁₁—, —COO—, —OCO—, —NR⁵²CO—, —NR⁵²SO₂—, —SO₂NR⁵²—, —NR⁵²COO—, —OCONR⁵²—, —NR⁵²CONR⁵³—, or —NR⁵²SO₂NR⁵³— where n11 is hydrogen or alkyl, R⁵² is hydrogen or alkyl, and R⁵¹ is hydrogen, alkyl, haloalkyl, alkoxyalkyl, hydroxyalkyl, cycloalkyl, cycloalkylalkyl, heterocycloalkyl, heterocycloalkylalkyl, aryl, aralkyl, heteroaryl, heteroaralkyl or heterocycloalkylalkyl or R⁵¹ together with R⁵² or R⁵³ in —SO₂NR⁵²—, —OCONR⁵²—, —NR⁵²CONR⁵³—, or —NR⁵²SO₂NR⁵³— form heterocycloalkyl] wherein the alkylene chain is optionally substituted with one to three halo atoms and the aromatic or alicyclic rings in R⁴⁶ are optionally substituted by one, two, or three R^{o} independently selected from alkyl, halo, hydroxy, alkoxy, hydroxyalkyl, alkoxyalkyl, haloalkyl, haloalkoxy, oxo, cyano, nitro, acyl, acyloxy, carboxy, alkoxycarbonyl, carbamoyl, alkylthio, alkylsulfinyl, alkylsulfonyl, alkoxycarbonylamino, alkylcarbamoyloxy, alkylsulfonylamino, aminosulfonyl, alkylaminosulfonyl, dialkylaminosulfonyl, aminocarbonyl, amino, monosubsituted or disubstituted amino and one R^{o} selected from aryl, aralkyl, heteroaryl, heteroaralkyl, cycloalkyl, cycloalkylalkyl, heterocycloalkyl, heterocycloalkylalkyl, aryloxy, benzyloxy, aryloxycarbonyl, arylthio, arylsulfonyl, arylsulfinyl, aryloxycarbonylamino, arylcarbamoyloxy, arylsulfonylamino, arylaminosulfonyl, or aralkylaminosulfonyl wherein the aromatic and alicyclic rings in R^{o} are optionally substituted with one, two, or three R^{p} wherein R^{p} is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, monosubstituted amino, dialkylamino, aryl, heteroaryl, cycloalkyl, carboxy, carboxamido, or alkoxycarbonyl;
R⁴⁷ is hydrogen, alkyl, hydroxyalkyl, alkoxyalkyl, cycloalkyl, cycloalkylalkyl, aryl, aralkyl, heteroaryl, heteroaralkyl, heterocycloalkyl, or heterocycloalkylalkyl; and
R⁴⁸ is hydrogen, alkyl, hydroxyalkyl, alkoxyalkyl, alkoxycarbonyl, aryloxycarbonyl, aralkyloxycarbonyl, heteroaryloxycarbonyl, heteroaralkyloxycarbonyl, alkylsulfonyl, arylsulfonyl, heteroarylsulfonyl, cycloalkyl, cycloalkylalkyl, aryl, aralkyl, heteroaryl, heteroaralkyl, heterocycloalkyl, or heterocycloalkylalkyl provided that one of R⁴⁷ and R⁴⁸ is other than hydrogen and wherein the aromatic or alicyclic rings in R⁴⁷ and R⁴⁸ are optionally substituted by one, two, or three R^{q} independently selected from alkyl, halo, hydroxy, hydroxyalkyl, alkoxy, alkoxyalkyl, haloalkyl, haloalkoxy, oxo, cyano, nitro, acyl, acyloxy, carboxy, alkoxycarbonyl, carbamoyl, alkylthio, alkylsulfinyl, alkylsulfonyl, alkoxycarbonylamino, alkylcarbamoyloxy, alkylsulfonylamino, aminosulfonyl, alkylaminosulfonyl, dialkylaminosulfonyl, amino, monosubsituted or disubstituted amino and one R^{q} selected from aryl, aralkyl, heteroaryl, heteroaralkyl, cycloalkyl, cycloalkylalkyl, heterocycloalkyl, heterocycloalkylalkyl, aryloxy, benzyloxy, aryloxycarbonyl, arylthio, arylsulfonyl, arylsulfinyl, aryloxycarbonylamino, arylcarbamoyloxy, arylsulfonylamino, arylaminosulfonyl, or aralkylaminosulfonyl wherein the aromatic and alicyclic rings in R^{q} are optionally substituted with one, two, or three R^{r} wherein R^{r} is independently selected from alkyl, halo, haloalkyl, haloalkoxy, hydroxy, nitro, cyano, hydroxyalkyl, alkoxy, alkoxyalkyl, aminoalkyl, alkylthio, alkylsulfonyl, amino, monosubstituted amino, dialkylamino, aryl, heteroaryl, cycloalkyl, carboxy, carboxamido, or alkoxycarbonyl; or
R^{3f} and R^{3g} are fluoro;
(l) 7-(2,2-dimethylpropyl)-6-thiophen-2-ylmethyl-7H-pyrrolo-[2,3-d]pyrimidine-2-carbonitrile;
(m) morpholine-4-carboxylic acid [(S)-1-(4-cyano-1-methylpiperidine-4-ylcarbamoyl)-4,4-dimethylhexyl]amide;
(n) morpholine-4-carboxylic acid [(S)-1-(4-cyano-1-propylpiperidine-4-ylcarbamoyl)-3,3,4,4-tetramethylpentyl]amide;
(o) morpholine-4-carboxylic acid [(S)-1-(4-cyano-1-propylpiperidine-4-ylcarbamoyl)-4,4-dimethylpentyl]amide;
(p) morpholine-4-carboxylic acid [(S)-1-(4-cyano-1-propylpiperidine-4-ylcarbamoyl)-4,4-dimethylhexyl]amide;
(q) morpholine-4-carboxylic acid [(R)-1-(4-cyano-1-methylpiperidine-4-ylcarbamoyl)-4,4-dimethylhexyl]amide;
(r) 5,5-dimethyl-2-(2-oxo-2H-benzo[e][1,3]oxazin-4-ylamino)heptanoic acid (4-cyano-1-propylpiperidin-4-yl)amide;
(l) 5,5-dimethyl-2-(2-oxo-2H-benzo[e][1,3]oxazin-4-ylamino)heptanoic acid (4-cyano-1-(3-morpholin-4-ylpropyl)piperidin-4-yl)amide;
(m) 5,5-dimethyl-2-(2-oxo-2H-benzo[e][1,3]oxazin-4-ylamino)heptanoic acid (4-cyano-1-(2-morpholin-4-ylethyl)piperidin-4-yl)amide;
(n) 5,5-dimethyl-2-(2-oxo-2H-benzo[e][1,3]oxazin-4-ylamino)heptanoic acid {4-cyano-1-[2-(2-methoxyethoxy)ethyl]piperidin-4-yl}amide;
(o) 5,5-dimethyl-2-(2-oxo-2H-benzo[e][1,3]oxazin-4-ylamino)heptanoic acid (4-cyano-1-methylpiperidin-4-yl)amide;
(p) 2-(7-fluoro-2-oxo-2H-benzo[e][1,3]oxazin-4-ylamino)-5,5-dimethylheptanoic acid (4-cyano-1-propylpiperidin-4-yl)amide;
(q) 2-(7-fluoro-2-oxo-2H-benzo[e][1,3]oxazin-4-ylamino)-5,5-dimethylhexanoic acid {4-cyano-1-(2-morpholin-4-ylethyl)piperidin-4-yl}amide; or
(r) 2-(7-fluoro-2-oxo-2H-benzo[e][1,3]oxazin-4-ylamino)-5,5-dimethylhexanoic acid {4-cyano-1-[2-(2-methoxyethoxy)ethyl]piperidin-4-yl}amide; or
a pharmaceutically acceptable salt thereof.

21. Use of a Cathepsin S inhibitor for the manufacture of a medicament for combination therapy with a biologic.

22. Use of a Cathepsin S inhibitor for the manufacture of a medicament for combination therapy with a biologic wherein the Cathepsin S inhibitor treats the immune response caused by the biologic.

23. The use of claim 21 or 22 wherein the biologic is a protein.

24. The use of claim 21 or 22 wherein the biologic is an antibody.

25. The use of claim 21 or 22 wherein the biologic is Remicade®, Refacto®, Referon-A®, Factor VIII, Factor VII, Betaseron®, Epogen®, Embrel®, Interferon beta, Botox®, Fabrazyme®, Elspar®, Cerezyme®, Myobloc®, Aldurazyme®, Verluma®, Interferon alpha, Humira®, Aranesp®, Zevalin® or OKT3.
